SEQUENCE LISTING (4 sequences)

SEQ ID NO: 1 - protein, 15 aa, Artificial
  Synthetic peptide
REVQSKIGYGRQIIS

SEQ ID NO: 2 - protein, 24 aa, Artificial
  Synthetic peptide
ELMTGELPYSHINNRDQIIFMVGR

SEQ ID NO: 3 - protein, 21 aa, Artificial
  Synthetic peptide
TGSLPYSHIGSRDQIIFMVGR

SEQ ID NO: 4 - protein, 18 aa, Artificial
  Synthetic peptide
GIVLYELMTGELPYSHIN

(19) United States
(12) Patent Application Publication
(10) Pub. No.: US 2010/0047840 A1
(43) Pub. Date: Feb. 25, 2010

(54) Polypeptide markers for the diagnosis of bladder cancer

(76) Inventors: Harald Mischak, Muellingen (DE); Stefan Wittke, Hannover (DE)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 11/991,035

(22) PCT Filed: Aug. 28, 2006
(86) PCT No.: PCT/EP2006/065742

§ 371 Date: Feb. 26, 2008

(30) Foreign Application Priority Data
Aug. 26, 2005 (EP) 05107840.0

(51) Int. Cl. C12Q 1/02; G01N 33/00

(52) U.S. Cl. 435/29; 436/86

(57) ABSTRACT

A method for the diagnosis of bladder cancer (BC) and/or for determining a tumor stage of bladder cancer, comprising the step of determining the presence or absence or amplitude of at least six polypeptide markers in a sample, wherein said polypeptide markers are selected from markers 1 to 836, which are characterized by the values for the molecular masses and migration times (CE time).

### BACKGROUND

1. Field of the Disclosure

The present disclosure relates to the use of the presence or absence or amplitude of one or more peptide markers in a sample from a subject for the (differential) diagnosis of bladder cancer and to a method for the diagnosis of bladder cancer, wherein the presence or absence or amplitude of the peptide marker or markers is indicative of the existence and of the tumor stage of bladder cancer.

2. Discussion of the Background Art

Bladder cancer is a malignant tumor on the mucous membrane of the bladder. Bladder cancer is one of the most common malignant diseases. In the urological field, it is the second most frequent cancer disease after prostate cancer. In the German-speaking area, there is an incidence of about 22 of 100,000 humans per year. In males, bladder cancer occurs about twice to three times as frequent as in females. Every year, an estimated 13,000 males and 5,000 females become afflicted with the disease in the Federal Republic of Germany. Bladder cancer is a disease of advanced age. The disease risk increases from the 40th year with increasing age.

### Diagnosis of Bladder Cancer:

A true early detection does not exist with bladder cancer. When there is blood in the urine or problems are encountered in micturition, it is urgently recommended to consult a physician quickly. Possibly, bladder cancer can be detected earlier thereby. If there is a suspicion of a neoplasm in the bladder, for example, if blood was observed in the urine or if there are continuing symptoms of bladder irritation, a cystoscopy is performed. When the examiner sees a tumor in the bladder wall, they can estimate which wall layers are penetrated by the tumor, and they can also take samples, which are then examined macroscopically. Depending on the tumor growth, a distinction is made between superficial and infiltrating (tissue-entering) carcinomas. The latter have already grown into the muscles of the bladder and can spread into the neighboring organs (for example, the prostate gland in males or the uterus in females). The histological-pathological classification of the mucous membrane tumors is effected according to the TNM system.

### Superficial Carcinomas:

pTa=non-invasive papillary carcinoma of the mucous membrane (urothelium)
pTcis=carcinoma in situ
pT1=infiltration below the mucous membrane (subepithelial connective tissue), subclassification pT1a-c

### Infiltrating Carcinomas:

pT2=infiltration of the muscular layer (muscularis propria), subclassification pT2a-b
pT3=growing beyond the muscular layer, subclassification pT3a-b
pT4=infiltration of neighboring organs, such as the prostate gland, uterus, vagina, pelvic wall

In addition, a radiological examination (urography) of the whole urinary tract can be performed. Supplementarily, the urine is examined for malignant cells under a microscope. For classification into stages, further diagnostic methods are employed, such as sonography, CT (computer tomography) or MRT (magnetic resonance tomography). By a sonographic examination of the abdomen, the position and size of a tumor can be established. In addition, such examination is used for examining the kidneys for an accumulation of urine, and the lymph nodes and liver are examined for metastases.

As described above, there is no functioning early detection of bladder cancer. To date, a clear diagnosis has been associated with invasive interventions, such as cystoscopy and a tissue biopsy. Thus, there was an object to provide a process and a method for as little invasive as possible, quick and low-cost diagnosis of bladder cancer, and for determining the tumor stage.

Vlahou et al., American Journal of Pathology 158 (2001), 1491-1501, describe the analysis of urine samples from patients with “transitional cell carcinoma” (TCC) using SELDI. Due to the low resolution of the SELDI technology, only a few markers can be detected in parallel thereby. This results in a low sensitivity and specificity of the diagnosis, which could not be validated in blinded tests.

W. Liu et al., in European Urology 47 (2005), 456-462, also describes a SELDI analysis of urine samples. The low number of markers found provides a poor specificity of the analysis.

Despite of the identical technologies employed by Vlahou et al. and Liu et al., different markers are found in the two works, which renders the non-existing validity of the procedure obvious. Also, the mass values stated for the markers are so imprecise that corresponding measurements are not reproducible, or the masses cannot be unambiguously assigned to individual substances.

It was the object of the present disclosure to overcome the mentioned drawbacks of the prior art, especially to define polypeptide markers which can be unambiguously assigned to individual peptides and are suitable for the diagnosis of bladder cancer and/or for determining the tumor stage.

### SUMMARY OF THE DISCLOSURE

Surprisingly, it has now been found that particular peptide markers in a sample from a subject can be used both for the diagnosis of bladder cancer and for evaluating the tumor stage of a bladder carcinoma.

Consequently, the present disclosure relates to the use of the presence or absence or amplitude of at least one polypeptide marker in a sample from a subject for the diagnosis of bladder cancer, wherein said polypeptide marker is selected from polypeptide marker Nos. 1 to 836 as characterized by the molecular masses and migration times as stated in Table 1.

TABLE 1
Polypeptide markers for the diagnosis of bladder cancer
and their molecular masses and migration times (CE time):

| | | CE |
|---|---|---|
| Number | Mass | Time |
| 1 | 4933.97 | 17.2 |
| 2 | 4712.25 | 14.3 |
| 3 | 1071.54 | 16.3 |
| 4 | 3273.42 | 17.7 |
| 5 | 1130.37 | 33.3 |
| 6 | 1180.53 | 33.7 |
| 7 | 980.36 | 34.0 |
| 8 | 11721.36 | 13.4 |
| 9 | 2077.12 | 16.8 |
| 10 | 840.42 | 19.3 |
| 11 | 2115.04 | 22.8 |
| 12 | 3385.43 | 21.1 |
| 13 | 4053.18 | 13.6 |
| 14 | 4240.13 | 14.2 |
| 15 | 1387.04 | 9.5 |
| 16 | 2597.47 | 18.6 |
| 17 | 2920.21 | 15.9 |
| 18 | 4509.09 | 25.1 |
| 19 | 8763.45 | 12.1 |
| 20 | 1535.72 | 26.3 |
| 21 | 911.28 | 31.9 |
| 22 | 1191.54 | 34.5 |
| 23 | 3759.90 | 12.8 |
| 24 | 2973.43 | 20.0 |
| 25 | 3325.67 | 15.4 |
| 26 | 10042.81 | 12.2 |
| 27 | 1380.67 | 19.3 |
| 28 | 3502.70 | 12.5 |
| 29 | 3632.63 | 15.9 |
| 30 | 8559.22 | 13.2 |
| 31 | 4960.86 | 15.6 |
| 32 | 858.41 | 18.9 |
| 33 | 847.40 | 20.0 |
| 34 | 980.51 | 18.7 |
| 35 | 1128.52 | 22.4 |
| 36 | 1407.68 | 35.6 |
| 37 | 2093.92 | 31.0 |
| 38 | 2421.04 | 32.5 |
| 39 | 1250.66 | 14.8 |
| 40 | 3242.20 | 18.0 |
| 41 | 1040.50 | 21.2 |
| 42 | 3176.40 | 11.9 |
| 43 | 2778.55 | 32.5 |
| 44 | 1422.58 | 35.8 |
| 45 | 3524.42 | 28.9 |
| 46 | 3831.74 | 24.8 |
| 47 | 2488.79 | 32.2 |
| 48 | 1099.51 | 24.9 |
| 49 | 1936.82 | 29.2 |
| 50 | 1265.61 | 23.4 |
| 51 | 3081.61 | 26.4 |
| 52 | 3266.48 | 26.5 |
| 53 | 3821.95 | 20.4 |
| 54 | 2407.10 | 24.0 |
| 55 | 1596.75 | 19.0 |
| 56 | 10341.75 | 18.1 |
| 57 | 4306.66 | 28.9 |
| 58 | 900.43 | 19.8 |
| 59 | 1645.74 | 15.9 |
| 60 | 1844.58 | 31.4 |
| 61 | 2130.97 | 29.6 |
| 62 | 2359.35 | 31.0 |
| 63 | 3343.58 | 28.5 |
| 64 | 3501.62 | 28.4 |
| 65 | 5675.31 | 19.0 |
| 66 | 1312.64 | 17.6 |
| 67 | 1867.68 | 30.4 |
| 68 | 2186.85 | 31.9 |
| 69 | 2414.51 | 33.6 |
| 70 | 2739.24 | 24.8 |
| 71 | 2907.36 | 33.9 |
| 72 | 3031.42 | 34.0 |
| 73 | 4345.89 | 30.6 |
| 74 | 6211.67 | 14.6 |
| 75 | 1080.52 | 22.9 |
| 76 | 1142.80 | 10.9 |
| 77 | 1321.74 | 10.6 |
| 78 | 1750.77 | 19.5 |
| 79 | 1989.90 | 29.3 |
| 80 | 2946.38 | 32.8 |
| 81 | 4942.34 | 21.4 |
| 82 | 1668.76 | 17.5 |
| 83 | 1688.71 | 14.5 |
| 84 | 1731.79 | 17.7 |
| 85 | 1822.74 | 27.4 |
| 86 | 3870.83 | 30.4 |
| 87 | 4275.48 | 20.6 |
| 88 | 1025.48 | 21.7 |
| 89 | 1353.67 | 22.1 |
| 90 | 1466.67 | 25.2 |
| 91 | 1588.73 | 26.7 |
| 92 | 1878.72 | 27.6 |
| 93 | 2933.31 | 24.1 |
| 94 | 3338.45 | 19.3 |
| 95 | 6055.63 | 15.6 |
| 96 | 2266.00 | 17.2 |
| 97 | 2939.05 | 31.1 |
| 98 | 3021.39 | 18.9 |
| 99 | 1299.60 | 17.6 |
| 100 | 1405.68 | 14.3 |
| 101 | 1526.72 | 19.3 |
| 102 | 1630.78 | 15.4 |
| 103 | 1865.83 | 30.0 |
| 104 | 2687.24 | 25.4 |
| 105 | 2756.27 | 32.8 |
| 106 | 1200.84 | 10.7 |
| 107 | 1391.81 | 10.7 |
| 108 | 1487.68 | 26.1 |
| 109 | 1919.87 | 23.3 |
| 110 | 2063.48 | 14.1 |
| 111 | 2753.47 | 34.2 |
| 112 | 2864.16 | 28.5 |
| 113 | 3858.65 | 21.9 |
| 114 | 4015.96 | 24.4 |
| 115 | 1324.60 | 16.3 |
| 116 | 1793.60 | 15.4 |
| 117 | 2215.79 | 30.7 |
| 118 | 1934.82 | 14.2 |
| 119 | 1935.96 | 13.6 |
| 120 | 2062.81 | 22.9 |
| 121 | 13372.32 | 25.9 |
| 122 | 1495.68 | 18.9 |
| 123 | 1513.49 | 35.4 |
| 124 | 1900.90 | 28.6 |
| 125 | 3108.94 | 33.8 |
| 126 | 1860.87 | 16.2 |
| 127 | 2194.68 | 14.3 |
| 128 | 3956.81 | 21.3 |
| 129 | 1496.70 | 26.5 |
| 130 | 3659.17 | 21.3 |
| 131 | 13169.24 | 25.6 |
| 132 | 1608.75 | 27.5 |
| 133 | 1707.73 | 11.1 |
| 134 | 1592.72 | 17.3 |
| 135 | 2029.80 | 15.0 |
| 136 | 13007.18 | 25.1 |
| 137 | 2117.77 | 29.9 |
| 138 | 2168.87 | 31.0 |
| 139 | 1916.86 | 31.3 |
| 140 | 2802.78 | 34.7 |
| 141 | 1111.77 | 10.8 |
| 142 | 1806.84 | 18.5 |
| 143 | 1594.71 | 26.1 |
| 144 | 1778.76 | 27.0 |
| 145 | 2582.95 | 19.0 |
| 146 | 3605.63 | 21.22 |
| 147 | 3765.31 | 42.77 |
| 148 | 4067.63 | 31.22 |
| 149 | 10640.4 | 19.68 |
| 150 | 2577.35 | 20.9 |
| 151 | 3292.55 | 37.5 |
| 152 | 2058.90 | 18.8 |
| 153 | 3001.36 | 33.2 |
| 154 | 2157.04 | 17.0 |
| 155 | 2854.36 | 32.6 |
| 156 | 1653.90 | 26.9 |
| 157 | 2658.32 | 13.1 |
| 158 | 3657.65 | 38.7 |
| 159 | 1754.92 | 27.8 |
| 160 | 1467.82 | 19.8 |
| 161 | 1580.91 | 20.1 |
| 162 | 2563.89 | 17.6 |
| 163 | 2355.15 | 17.9 |
| 164 | 3891.40 | 20.2 |
| 165 | 1561.49 | 35.6 |
| 166 | 1576.63 | 23.1 |
| 167 | 1911.07 | 20.8 |
| 168 | 3265.47 | 19.8 |
| 169 | 2344.91 | 32.3 |
| 170 | 2743.98 | 32.8 |
| 171 | 3401.75 | 18.1 |
| 172 | 3425.87 | 26.1 |
| 173 | 5510.18 | 23.1 |
| 174 | 2356.70 | 33.5 |
| 175 | 3256.25 | 29.3 |
| 176 | 12716.23 | 23.1 |
| 177 | 1050.49 | 23.3 |
| 178 | 1950.87 | 33.9 |
| 179 | 1649.76 | 17.9 |
| 180 | 1312.55 | 26.3 |
| 181 | 1438.47 | 35.2 |
| 182 | 3248.51 | 26.8 |
| 183 | 2742.26 | 25.3 |
| 184 | 981.60 | 20.7 |
| 185 | 1679.97 | 19.3 |
| 186 | 801.43 | 22.7 |
| 187 | 816.41 | 21.05 |
| 188 | 818.48 | 21.95 |
| 189 | 838.36 | 25.12 |
| 190 | 838.44 | 35.05 |
| 191 | 840.47 | 19.68 |
| 192 | 852.47 | 20.42 |
| 193 | 868.45 | 23.35 |
| 194 | 871.48 | 21.11 |
| 195 | 875.51 | 21.83 |
| 196 | 876.43 | 35.13 |
| 197 | 883.45 | 23.27 |
| 198 | 884.36 | 24.91 |
| 199 | 892.32 | 35.22 |
| 200 | 906.34 | 34.93 |
| 201 | 906.51 | 21.89 |
| 202 | 912.55 | 20.04 |
| 203 | 914.49 | 23.75 |
| 204 | 915.28 | 35.24 |
| 205 | 931.51 | 19.99 |
| 206 | 935.49 | 23.76 |
| 207 | 936.49 | 21.43 |
| 208 | 939.51 | 23.79 |
| 209 | 941.46 | 24.85 |
| 210 | 944.55 | 21.35 |
| 211 | 945.46 | 25.7 |
| 212 | 947.5 | 24.75 |
| 213 | 951.55 | 20.32 |
| 214 | 956.48 | 20.38 |
| 215 | 958.51 | 20.76 |
| 216 | 963.54 | 21.65 |
| 217 | 973.28 | 35.58 |
| 218 | 984.49 | 24.43 |
| 219 | 988.55 | 35.81 |
| 220 | 988.55 | 21.83 |
| 221 | 994.49 | 25.17 |
| 222 | 995.44 | 25.15 |
| 223 | 996.51 | 24.63 |
| 224 | 1009.47 | 20.96 |
| 225 | 1012.53 | 35.08 |
| 226 | 1016.31 | 35.67 |
| 227 | 1016.49 | 25.81 |
| 228 | 1027.55 | 24.6 |
| 229 | 1031.5 | 25.66 |
| 230 | 1037.57 | 21.98 |
| 231 | 1038.61 | 20.84 |
| 232 | 1042.54 | 25.13 |
| 233 | 1053.53 | 25.08 |
| 234 | 1056.57 | 25.49 |
| 235 | 1066.52 | 25.96 |
| 236 | 1068.56 | 21.69 |
| 237 | 1073.36 | 35.39 |
| 238 | 1078.69 | 19.67 |
| 239 | 1082.55 | 24.37 |
| 240 | 1083.56 | 21.65 |
| 241 | 1096.41 | 35.91 |
| 242 | 1096.53 | 26.12 |
| 243 | 1098.56 | 21.42 |
| 244 | 1099.56 | 21.56 |
| 245 | 1100.59 | 21.62 |
| 246 | 1101.54 | 22.21 |
| 247 | 1106.52 | 26.02 |
| 248 | 1109.68 | 21.06 |
| 249 | 1114.54 | 25.52 |
| 250 | 1114.57 | 21.27 |
| 251 | 1124.58 | 21.04 |
| 252 | 1125.55 | 24.9 |
| 253 | 1125.58 | 21.77 |
| 254 | 1126.52 | 21.2 |
| 255 | 1126.57 | 25.61 |
| 256 | 1134.63 | 23.68 |
| 257 | 1135.53 | 27.51 |
| 258 | 1136.59 | 20.27 |
| 259 | 1137.65 | 24.08 |
| 260 | 1138.64 | 19.55 |
| 261 | 1139.54 | 21.01 |
| 262 | 1140.53 | 21.14 |
| 263 | 1141.57 | 25.2 |
| 264 | 1142.61 | 21.8 |
| 265 | 1143.56 | 36.95 |
| 266 | 1144.43 | 35.57 |
| 267 | 1144.6 | 26.25 |
| 268 | 1150.61 | 22.42 |
| 269 | 1153.38 | 35.68 |
| 270 | 1153.57 | 26.34 |
| 271 | 1154.57 | 36.72 |
| 272 | 1155.55 | 20.77 |
| 273 | 1157.58 | 37.41 |
| 274 | 1159.63 | 20.75 |
| 275 | 1159.64 | 26.09 |
| 276 | 1160.6 | 26.14 |
| 277 | 1162.59 | 20.26 |
| 278 | 1165.55 | 37.15 |
| 279 | 1166.62 | 22.8 |
| 280 | 1168.58 | 26.23 |
| 281 | 1169.61 | 23.85 |
| 282 | 1170.64 | 21.61 |
| 283 | 1171.62 | 23.34 |
| 284 | 1173.58 | 37.51 |
| 285 | 1179.65 | 20.06 |
| 286 | 1181.62 | 26.06 |
| 287 | 1190.64 | 22.02 |
| 288 | 1196.37 | 36.14 |
| 289 | 1196.59 | 21.07 |
| 290 | 1198.6 | 26.05 |
| 291 | 1199.63 | 21.92 |
| 292 | 1200.59 | 24.21 |
| 293 | 1201.59 | 24.85 |
| 294 | 1204.65 | 21.93 |
| 295 | 1209.58 | 26.26 |
| 296 | 1210.45 | 36.58 |
| 297 | 1210.61 | 20.92 |
| 298 | 1213.62 | 27.75 |
| 299 | 1215.49 | 27.61 |
| 300 | 1216.59 | 24.27 |
| 301 | 1217.58 | 35.91 |
| 302 | 1218.42 | 36.88 |
| 303 | 1219.62 | 26.92 |
| 304 | 1220.63 | 22.11 |
| 305 | 1221.63 | 26.86 |
| 306 | 1223.6 | 27.47 |
| 307 | 1226.58 | 21 |
| 308 | 1226.63 | 23.47 |
| 309 | 1230.6 | 21.74 |
| 310 | 1234.42 | 36.16 |
| 311 | 1234.61 | 27.41 |
| 312 | 1238.55 | 36.74 |
| 313 | 1238.63 | 21.86 |
| 314 | 1239.52 | 34.91 |
| 315 | 1241.6 | 26.68 |
| 316 | 1245.6 | 21.63 |
| 317 | 1247.58 | 22.02 |
| 318 | 1250.52 | 35.35 |
| 319 | 1257.63 | 26.86 |
| 320 | 1260.61 | 21.84 |
| 321 | 1263.6 | 22.72 |
| 322 | 1267.64 | 21.55 |
| 323 | 1281.64 | 27.16 |
| 324 | 1284.62 | 28.22 |
| 325 | 1284.69 | 21.88 |
| 326 | 1287.65 | 21.85 |
| 327 | 1296.65 | 19.37 |
| 328 | 1300.65 | 23.07 |
| 329 | 1304.66 | 21.85 |
| 330 | 1309.74 | 21.97 |
| 331 | 1310.64 | 27.14 |
| 332 | 1321.63 | 37.98 |
| 333 | 1321.65 | 28.4 |
| 334 | 1328.64 | 26.87 |
| 335 | 1338.66 | 23.99 |
| 336 | 1340.65 | 28.33 |
| 337 | 1349.48 | 36.47 |
| 338 | 1349.77 | 20.22 |
| 339 | 1350.65 | 21.56 |
| 340 | 1352.62 | 29.68 |
| 341 | 1352.83 | 24.47 |
| 342 | 1353.65 | 21.48 |
| 343 | 1358.44 | 36.46 |
| 344 | 1361.69 | 21.95 |
| 345 | 1362.72 | 22.13 |
| 346 | 1366.68 | 27.48 |
| 347 | 1368.68 | 21.7 |
| 348 | 1375.67 | 23.28 |
| 349 | 1378.67 | 28.85 |
| 350 | 1383.69 | 39.02 |
| 351 | 1390.5 | 37.05 |
| 352 | 1392.68 | 21.75 |
| 353 | 1395.71 | 22.59 |
| 354 | 1396.5 | 37.63 |
| 355 | 1396.68 | 28.05 |
| 356 | 1413.62 | 25.78 |
| 357 | 1417.69 | 20.01 |
| 358 | 1421.58 | 37.51 |
| 359 | 1423.68 | 21.47 |
| 360 | 1425.65 | 22.34 |
| 361 | 1436.79 | 22.82 |
| 362 | 1438.72 | 21.98 |
| 363 | 1439.72 | 29.7 |
| 364 | 1440.72 | 39.19 |
| 365 | 1440.75 | 21.63 |
| 366 | 1442.68 | 27.76 |
| 367 | 1444.49 | 36.69 |
| 368 | 1448.83 | 20.53 |
| 369 | 1449.7 | 21.84 |
| 370 | 1451.71 | 29.19 |
| 371 | 1452.72 | 23.55 |
| 372 | 1456.75 | 22.48 |
| 373 | 1457.68 | 22 |
| 374 | 1458.69 | 28.03 |
| 375 | 1460.71 | 20.31 |
| 376 | 1473.7 | 22.3 |
| 377 | 1474.75 | 19.94 |
| 378 | 1483.72 | 22.59 |
| 379 | 1487.71 | 29.58 |
| 380 | 1491.8 | 39.86 |
| 381 | 1493.74 | 22.11 |
| 382 | 1507.8 | 39.98 |
| 383 | 1508.77 | 23.17 |
| 384 | 1509.59 | 37.07 |
| 385 | 1510.72 | 28.39 |
| 386 | 1510.75 | 20.11 |
| 387 | 1522.78 | 22.81 |
| 388 | 1523.89 | 29.75 |
| 389 | 1524.72 | 20.02 |
| 390 | 1535.75 | 22.03 |
| 391 | 1540.81 | 39.97 |
| 392 | 1549.76 | 39.5 |
| 393 | 1552.57 | 37.35 |
| 394 | 1556.8 | 40.03 |
| 395 | 1560.69 | 22.08 |
| 396 | 1562.76 | 22.48 |
| 397 | 1563.77 | 29.56 |
| 398 | 1564.78 | 28.53 |
| 399 | 1567.77 | 20.2 |
| 400 | 1576.74 | 46.02 |
| 401 | 1577.75 | 40.06 |
| 402 | 1579.75 | 20.05 |
| 403 | 1579.78 | 29.83 |
| 404 | 1609.81 | 30.25 |
| 405 | 1610.91 | 30.12 |
| 406 | 1611.83 | 40.12 |
| 407 | 1611.89 | 23.27 |
| 408 | 1613.89 | 23.98 |
| 409 | 1619.78 | 22.6 |
| 410 | 1620.74 | 29.58 |
| 411 | 1620.79 | 22.71 |
| 412 | 1624.61 | 37.73 |
| 413 | 1631.78 | 47.02 |
| 414 | 1632.79 | 38.03 |
| 415 | 1633.83 | 19.58 |
| 416 | 1635.83 | 30.38 |
| 417 | 1636.79 | 20.1 |
| 418 | 1636.81 | 30.33 |
| 419 | 1638.8 | 20.24 |
| 420 | 1640.75 | 28.28 |
| 421 | 1658.68 | 21.55 |
| 422 | 1658.79 | 30.04 |
| 423 | 1660.76 | 37.7 |
| 424 | 1664.82 | 29.87 |
| 425 | 1668.87 | 40.47 |
| 426 | 1673.82 | 29.4 |
| 427 | 1674.83 | 20.71 |
| 428 | 1680.82 | 30.02 |
| 429 | 1684.74 | 31.42 |
| 430 | 1684.81 | 39.43 |
| 431 | 1689.86 | 27.62 |
| 432 | 1693.83 | 20.48 |
| 433 | 1693.84 | 23.48 |
| 434 | 1697.81 | 30.86 |
| 435 | 1698.67 | 37.53 |
| 436 | 1705.78 | 46.77 |
| 437 | 1708.85 | 31.08 |
| 438 | 1725.66 | 38.3 |
| 439 | 1728.84 | 36.74 |
| 440 | 1729.6 | 53.46 |
| 441 | 1737.85 | 31.04 |
| 442 | 1746.95 | 19.75 |
| 443 | 1749.88 | 30.61 |
| 444 | 1761.91 | 21.61 |
| 445 | 1764.86 | 29.97 |
| 446 | 1767.07 | 24.12 |
| 447 | 1768.9 | 20.78 |
| 448 | 1769.78 | 28.25 |
| 449 | 1773.87 | 34.89 |
| 450 | 1782.91 | 25.93 |
| 451 | 1783.86 | 39.94 |
| 452 | 1784.88 | 40.04 |
| 453 | 1786.66 | 38.34 |
| 454 | 1788.89 | 30.26 |
| 455 | 1794.84 | 30.49 |
| 456 | 1796.85 | 30.86 |
| 457 | 1798.79 | 37.33 |
| 458 | 1807.91 | 20.7 |
| 459 | 1813.78 | 31.79 |
| 460 | 1818.9 | 31.1 |
| 461 | 1821.88 | 30.43 |
| 462 | 1823.99 | 24.61 |
| 463 | 1825.87 | 31.81 |
| 464 | 1829.03 | 21.24 |
| 465 | 1834.9 | 31.15 |
| 466 | 1835.92 | 24.04 |
| 467 | 1838.89 | 27.03 |
| 468 | 1846.93 | 32.08 |
| 469 | 1852.96 | 25.14 |
| 470 | 1858.91 | 24.21 |
| 471 | 1859.9 | 24.38 |
| 472 | 1860.42 | 33.82 |
| 473 | 1863.95 | 44.05 |
| 474 | 1880.97 | 44.03 |
| 475 | 1885.96 | 21.26 |
| 476 | 1889.94 | 33.12 |
| 477 | 1891.65 | 54.28 |
| 478 | 1892.95 | 22.4 |
| 479 | 1893.06 | 24.64 |
| 480 | 1899.93 | 21.64 |
| 481 | 1911.05 | 30.74 |
| 482 | 1912.74 | 32.7 |
| 483 | 1916.85 | 20.35 |
| 484 | 1926.91 | 30.99 |
| 485 | 1927.99 | 19.53 |
| 486 | 1932.98 | 21.65 |
| 487 | 1933.95 | 21.63 |
| 488 | 1936.11 | 20.51 |
| 489 | 1938.96 | 21.33 |
| 490 | 1942.77 | 36.19 |
| 491 | 1945.07 | 33.71 |
| 492 | 1946.95 | 31.75 |
| 493 | 1949.97 | 21.66 |
| 494 | 1952.42 | 25.65 |
| 495 | 1955.04 | 25.29 |
| 496 | 1955.94 | 28.14 |
| 497 | 1969.43 | 32.31 |
| 498 | 1976.96 | 32.36 |
| 499 | 1977.99 | 32.21 |
| 500 | 1989.02 | 24.75 |
| 501 | 1992.02 | 22.05 |
| 502 | 1993.02 | 27.07 |
| 503 | 1993.96 | 32.2 |
| 504 | 1997.99 | 25.13 |
| 505 | 2004.02 | 24.63 |
| 506 | 2006.93 | 33 |
| 507 | 2008.03 | 22.13 |
| 508 | 2021.06 | 21.98 |
| 509 | 2022.97 | 33.39 |
| 510 | 2023.99 | 21.47 |
| 511 | 2026.1 | 25.66 |
| 512 | 2031 | 21.8 |
| 513 | 2035.07 | 40.19 |
| 514 | 2040.02 | 32.87 |
| 515 | 2042.14 | 25.21 |
| 516 | 2057.04 | 24.85 |
| 517 | 2065 | 24.48 |
| 518 | 2076.61 | 32.44 |
| 519 | 2078.05 | 22.47 |
| 520 | 2081.03 | 20.17 |
| 521 | 2087.96 | 19.46 |
| 522 | 2088.04 | 32.92 |
| 523 | 2088.95 | 23.68 |
| 524 | 2090.04 | 39.48 |
| 525 | 2095.53 | 24.83 |
| 526 | 2100.12 | 19.6 |
| 527 | 2110 | 24.11 |
| 528 | 2114.07 | 31.61 |
| 529 | 2118.04 | 27.72 |
| 530 | 2129.06 | 26.97 |
| 531 | 2134.04 | 27.78 |
| 532 | 2138.03 | 21.78 |
| 533 | 2146 | 52.45 |
| 534 | 2188.03 | 39.71 |
| 535 | 2192.08 | 22.16 |
| 536 | 2195.49 | 33.36 |
| 537 | 2197.94 | 34.23 |
| 538 | 2199.08 | 22.31 |
| 539 | 2207.16 | 43.61 |
| 540 | 2208 | 32.37 |
| 541 | 2208.97 | 31.75 |
| 542 | 2211.11 | 20.28 |
| 543 | 2212.06 | 33.53 |
| 544 | 2219.56 | 33.59 |
| 545 | 2227.05 | 33.42 |
| 546 | 2233.1 | 22.49 |
| 547 | 2237.06 | 27.1 |
| 548 | 2244.17 | 19.48 |
| 549 | 2248.75 | 22.52 |
| 550 | 2251.77 | 33.11 |
| 551 | 2264.12 | 22.67 |
| 552 | 2269.29 | 38.14 |
| 553 | 2279.57 | 23.77 |
| 554 | 2282.13 | 22.29 |
| 555 | 2283.12 | 26.51 |
| 556 | 2292.21 | 21.29 |
| 557 | 2302.38 | 26.16 |
| 558 | 2305.7 | 34.8 |
| 559 | 2310.15 | 41.24 |
| 560 | 2315.22 | 19.45 |
| 561 | 2318.29 | 26.28 |
| 562 | 2319.19 | 33.87 |
| 563 | 2320.79 | 22.48 |
| 564 | 2322.17 | 22.28 |
| 565 | 2323.14 | 22.39 |
| 566 | 2328.06 | 20.83 |
| 567 | 2332.08 | 50.2 |
| 568 | 2335.75 | 26.61 |
| 569 | 2339.08 | 34.01 |
| 570 | 2339.17 | 22.63 |
| 571 | 2343.49 | 20.08 |
| 572 | 2361.19 | 20.78 |
| 573 | 2367.48 | 34.22 |
| 574 | 2371.18 | 22.77 |
| 575 | 2377.19 | 20.79 |
| 576 | 2380.16 | 36.49 |
| 577 | 2389.33 | 22.4 |
| 578 | 2392.8 | 35.28 |
| 579 | 2394 | 23.93 |
| 580 | 2405.6 | 22.19 |
| 581 | 2410.16 | 23.45 |
| 582 | 2414.26 | 19.58 |
| 583 | 2430.19 | 28.28 |
| 584 | 2430.72 | 35.39 |
| 585 | 2436.15 | 22.8 |
| 586 | 2437.22 | 22.86 |
| 587 | 2445.8 | 28.15 |
| 588 | 2451.59 | 24.02 |
| 589 | 2471.25 | 34.76 |
| 590 | 2478.22 | 22.45 |
| 591 | 2479.15 | 48.79 |
| 592 | 2485.16 | 34.39 |
| 593 | 2501.21 | 34.37 |
| 594 | 2502.73 | 22.84 |
| 595 | 2507.21 | 22.78 |
| 596 | 2518.4 | 22.8 |
| 597 | 2525.21 | 27.72 |
| 598 | 2534.56 | 22.81 |
| 599 | 2548.38 | 35.1 |
| 600 | 2551.32 | 34.75 |
| 601 | 2553.52 | 34.27 |
| 602 | 2557.15 | 28.17 |
| 603 | 2559.25 | 19.44 |
| 604 | 2565.18 | 23.74 |
| 605 | 2567.28 | 28.17 |
| 606 | 2570.28 | 42.66 |
| 607 | 2574.08 | 32.92 |
| 608 | 2580.19 | 23 |
| 609 | 2587.39 | 21.17 |
| 610 | 2599.26 | 28.18 |
| 611 | 2607.6 | 34.4 |
| 612 | 2616 | 28.34 |
| 613 | 2627.08 | 23.44 |
| 614 | 2632.18 | 24.96 |
| 615 | 2639.41 | 20.98 |
| 616 | 2668.46 | 42.02 |
| 617 | 2682.22 | 22.49 |
| 618 | 2702.1 | 38.01 |
| 619 | 2710.58 | 35.15 |
| 620 | 2723.92 | 23.81 |
| 621 | 2726.32 | 28.87 |
| 622 | 2726.37 | 42.94 |
| 623 | 2733.84 | 34.18 |
| 624 | 2742.36 | 42.12 |
| 625 | 2748.87 | 36.35 |
| 626 | 2754.36 | 29.64 |
| 627 | 2761.4 | 21.49 |
| 628 | 2777.61 | 21.54 |
| 629 | 2796.02 | 21 |
| 630 | 2810.46 | 36.66 |
| 631 | 2816.5 | 28.41 |
| 632 | 2821.19 | 23.73 |
| 633 | 2837.91 | 23.99 |
| 634 | 2839.42 | 24.17 |
| 635 | 2853.32 | 23.8 |
| 636 | 2861.32 | 50.73 |
| 637 | 2887.42 | 35.66 |
| 638 | 2889.19 | 24.09 |
| 639 | 2889.65 | 28.73 |
| 640 | 2903.72 | 35.61 |
| 641 | 2911 | 21.68 |
| 642 | 2914.57 | 24.28 |
| 643 | 2917.41 | 29.03 |
| 644 | 2924.58 | 22.97 |
| 645 | 2926.2 | 22.16 |
| 646 | 2932.36 | 34.11 |
| 647 | 2942.4 | 22.22 |
| 648 | 2977.61 | 29.14 |
| 649 | 2982.9 | 22.24 |
| 650 | 2989.4 | 24.42 |
| 651 | 2999.23 | 22.22 |
| 652 | 3002.47 | 23.95 |
| 653 | 3011.46 | 29.72 |
| 654 | 3013.27 | 22.28 |
| 655 | 3017.95 | 49.05 |
| 656 | 3023.33 | 24.55 |
| 657 | 3025.69 | 29.85 |
| 658 | 3035 | 42.23 |
| 659 | 3035.17 | 47.18 |
| 660 | 3038.11 | 22.42 |
| 661 | 3041.47 | 29.95 |
| 662 | 3044.98 | 34.34 |
| 663 | 3047.64 | 26.68 |
| 664 | 3056.94 | 45.28 |
| 665 | 3064.36 | 20.55 |
| 666 | 3076.33 | 19.64 |
| 667 | 3080.99 | 29.92 |
| 668 | 3091.41 | 28.33 |
| 669 | 3092.45 | 30.97 |
| 670 | 3092.51 | 36.23 |
| 671 | 3098.49 | 30.07 |
| 672 | 3112.42 | 22.44 |
| 673 | 3113.81 | 20.39 |
| 674 | 3114.66 | 30.3 |
| 675 | 3121.31 | 30.23 |
| 676 | 3129.86 | 35.93 |
| 677 | 3136.55 | 24.5 |
| 678 | 3139.44 | 29.48 |
| 679 | 3143.51 | 32.87 |
| 680 | 3145.49 | 38.8 |
| 681 | 3152.23 | 24.54 |
| 682 | 3158.6 | 29.69 |
| 683 | 3168.38 | 24.69 |
| 684 | 3178.26 | 30.25 |
| 685 | 3184.67 | 24.91 |
| 686 | 3193.27 | 22.63 |
| 687 | 3195.2 | 20.64 |
| 688 | 3202.2 | 30.52 |
| 689 | 3204.32 | 35.31 |
| 690 | 3219.43 | 35.05 |
| 691 | 3223.42 | 24.74 |
| 692 | 3240.47 | 25.2 |
| 693 | 3249.16 | 22.61 |
| 694 | 3258.51 | 22.89 |
| 695 | 3260.4 | 41.55 |
| 696 | 3261.35 | 22.17 |
| 697 | 3265.33 | 36.02 |
| 698 | 3271.39 | 30.75 |
| 699 | 3276.25 | 33.59 |
| 700 | 3282.79 | 30.09 |
| 701 | 3287.39 | 30.84 |
| 702 | 3290.39 | 24.1 |
| 703 | 3295.51 | 25.38 |
| 704 | 3298.48 | 36.06 |
| 705 | 3310.26 | 36.5 |
| 706 | 3314.41 | 20.12 |
| 707 | 3318.68 | 30.98 |
| 708 | 3325.9 | 54.11 |
| 709 | 3333.28 | 19.42 |
| 710 | 3333.8 | 23.85 |
| 711 | 3334.27 | 31 |
| 712 | 3336.48 | 39.43 |
| 713 | 3341.91 | 21.05 |
| 714 | 3356.38 | 25.56 |
| 715 | 3359.67 | 31.88 |
| 716 | 3363.69 | 30.21 |
| 717 | 3375.69 | 31.9 |
| 718 | 3386.16 | 22.7 |
| 719 | 3400.07 | 42.03 |
| 720 | 3416.82 | 36.78 |
| 721 | 3435.94 | 22.7 |
| 722 | 3457.46 | 35.83 |
| 723 | 3457.73 | 31.51 |
| 724 | 3472.36 | 21.75 |
| 725 | 3510.65 | 40.23 |
| 726 | 3521.07 | 30.74 |
| 727 | 3530.63 | 26.11 |
| 728 | 3531.46 | 30.16 |
| 729 | 3546.82 | 26.7 |
| 730 | 3559.71 | 24.89 |
| 731 | 3571.61 | 31.03 |
| 732 | 3582.7 | 19.47 |
| 733 | 3583.5 | 41.45 |
| 734 | 3590.72 | 29.01 |
| 735 | 3596.51 | 21.57 |
| 736 | 3603.92 | 20.21 |
| 737 | 3633.02 | 33.15 |
| 738 | 3633.71 | 27.02 |
| 739 | 3635.47 | 31.87 |
| 740 | 3669.71 | 24.13 |
| 741 | 3686.04 | 22.21 |
| 742 | 3696.88 | 26.94 |
| 743 | 3702.45 | 32.45 |
| 744 | 3703.85 | 22.51 |
| 745 | 3706.68 | 22 |
| 746 | 3710.86 | 19.6 |
| 747 | 3737.71 | 37.15 |
| 748 | 3738.58 | 24.74 |
| 749 | 3765.51 | 20.21 |
| 750 | 3774.84 | 22.9 |
| 751 | 3775.25 | 27 |
| 752 | 3775.9 | 25.28 |
| 753 | 3790.98 | 32.88 |
| 754 | 3816.45 | 21.94 |
| 755 | 3839.91 | 19.7 |
| 756 | 3864.66 | 33.71 |
| 757 | 3906.75 | 24.13 |
| 758 | 3935.56 | 34.14 |
| 759 | 3948.83 | 45.72 |
| 760 | 3968.68 | 21.11 |
| 761 | 4002.73 | 20.66 |
| 762 | 4008.95 | 23.57 |
| 763 | 4025.43 | 33.23 |
| 764 | 4042.93 | 33.13 |
| 765 | 4044.98 | 26.39 |
| 766 | 4069.65 | 25.2 |
| 767 | 4081.58 | 24.53 |
| 768 | 4083.75 | 31.32 |
| 769 | 4097.98 | 24.6 |
| 770 | 4113.77 | 24.46 |
| 771 | 4122.1 | 24.06 |
| 772 | 4124.05 | 21.49 |
| 773 | 4143.81 | 26.55 |
| 774 | 4196.47 | 20.96 |
| 775 | 4215.52 | 26.95 |
| 776 | 4217.98 | 26 |
| 777 | 4229.11 | 29.06 |
| 778 | 4278.77 | 23.34 |
| 779 | 4306 | 25.13 |
| 780 | 4322.14 | 25.18 |
| 781 | 4339 | 25.4 |
| 782 | 4352.98 | 20.17 |
| 783 | 4363.14 | 23.05 |
| 784 | 4369.07 | 20.21 |
| 785 | 4378.88 | 23.14 |
| 786 | 4381.08 | 34.13 |
| 787 | 4395.06 | 23.09 |
| 788 | 4415.72 | 22.53 |
| 789 | 4426.27 | 20.09 |
| 790 | 4436.04 | 26.25 |
| 791 | 4441.22 | 22.43 |
| 792 | 4483.33 | 25.4 |
| 793 | 4491.91 | 26.23 |
| 794 | 4538.61 | 26.35 |
| 795 | 4548.93 | 26.45 |
| 796 | 4550.95 | 23.31 |
| 797 | 4567.27 | 23.29 |
| 798 | 4583.7 | 35.76 |
| 799 | 4619.43 | 25.07 |
| 800 | 4637.97 | 25.75 |
| 801 | 4805.62 | 26.47 |
| 802 | 4817.33 | 23.86 |
| 803 | 4833.29 | 23.89 |
| 804 | 4849.07 | 23.86 |
| 805 | 4863.23 | 26.67 |
| 806 | 4864.81 | 23.88 |
| 807 | 4933.46 | 20.58 |
| 808 | 4976.46 | 20.62 |
| 809 | 5027.18 | 26.53 |
| 810 | 5039.24 | 25.56 |
| 811 | 5043.12 | 26.54 |
| 812 | 5090.45 | 20.1 |
| 813 | 5160.13 | 21.74 |
| 814 | 5229.59 | 25.1 |
| 815 | 5276.59 | 20.04 |
| 816 | 5495.5 | 19.52 |
| 817 | 5574.44 | 23.22 |
| 818 | 6186.07 | 24.91 |
| 819 | 6237.1 | 21.07 |
| 820 | 6491.14 | 19.44 |
| 821 | 6650.84 | 25.52 |
| 822 | 7907.23 | 19.59 |
| 823 | 7958.72 | 34.38 |
| 824 | 8289.54 | 19.54 |
| 825 | 8445.41 | 19.52 |
| 826 | 8837.64 | 21.06 |
| 827 | 8853.9 | 21.1 |
| 828 | 8917.52 | 22.55 |
| 829 | 10199.94 | 21.11 |
| 830 | 10753.72 | 19.71 |
| 831 | 10770.19 | 19.64 |
| 832 | 11041.95 | 21.98 |
| 833 | 11738.36 | 19.6 |
| 834 | 11967.98 | 20.48 |
| 835 | 14111.34 | 21.97 |
| 836 | 3266.45 | 22.73 |

### DETAILED DESCRIPTION OF THE PREFERRED EMBODIMENT

With the present disclosure, it is possible to diagnose bladder cancer at a very early stage. Thus, the disease can be cured by known methods at an early stage. The disclosure further enables an inexpensive, quick and reliable evaluation of the tumor stage and a diagnosis with in part non-invasive or only minimal-invasive operations.

The migration time is determined by capillary electrophoresis (CE), for example, as set forth in the Example under item 2. In this Example, a glass capillary of 90 cm in length and with an inner diameter (ID) of 50 μm and an outer diameter (OD) of 360 μm is operated at an applied voltage of 30 kV. As the mobile solvent, for example, 300% methanol, 0.5% formic acid in water is used.

It is known that the CE migration times may vary. Nevertheless, the order in which the polypeptide markers are eluted is typically the same under the stated conditions for any CE system employed. In order to balance any differences in the migration time that may nevertheless occur, the system can be normalized using standards for which the migration times are exactly known. These standards may be, for example, the polypeptides stated in the Examples (see the Example, item 3).

The characterization of the polypeptides shown in Tables 1 to 8 was determined by means of capillary electrophoresis-mass spectrometry (CE-MS), a method which has been described in detail, for example, by Neuhoff et al. (Rapid communications in mass spectrometry, 2004, Vol. 20, pages 149-156). The variation of the molecular masses between individual measurements or between different mass spectrometers is relatively small when the calibration is exact, typically within a range of ±0.10%, preferably within a range of ±0.050%, more preferably ±0.030%.

The polypeptide markers according to the disclosure are proteins or peptides or degradation products of proteins or peptides. They may be chemically modified, for example, by posttranslational modifications, such as glycosylation, phosphorylation, alkylation or disulfide bridges, or by other reactions, for example, within the scope of degradation. In addition, the polypeptide markers may also be chemically altered, for example, oxidized, during the purification of the samples.

Proceeding from the parameters that determine the polypeptide markers (molecular weight and migration time), it is possible to identify the sequence of the corresponding polypeptides by methods known in the prior art.

The polypeptides according to the disclosure (see Tables 1 to 8) are used to diagnose bladder cancer, on the one hand, and to enable a distinction between different tumor stages, on the other. “Diagnosis” means the process of knowledge gaining by assigning symptoms or phenomena to a disease or injury. In the present case, the existence of bladder cancer and the tumor stage of the bladder carcinoma is concluded from the presence or absence or amplitude of particular polypeptide markers. Thus, the polypeptide markers according to the disclosure are determined in a sample from a subject, wherein their presence or absence allows to conclude the existence of bladder cancer in the case of frequency markers, or the difference in signal intensities allows to conclude the existence of bladder cancer in the case of amplitude markers. The presence or absence or amplitude of a polypeptide marker can be measured by any method known in the prior art. Methods which may be used are exemplified below.

A polypeptide marker is considered present if its measured value is at least as high as its threshold value. If the measured value is lower, then the polypeptide marker is considered absent. The threshold value can be determined either by the sensitivity of the measuring method (detection limit) or defined from experience.

In the context of the present disclosure, the threshold value is considered to be exceeded preferably if the measured value of the sample for a certain molecular mass is at least twice as high as that of a blank sample (for example, only buffer or solvent).

The polypeptide marker or markers is/are used in such a way that its/their presence or absence is measured, wherein the presence or absence is indicative of bladder cancer (frequency markers). Thus, there are polypeptide markers which are typically present in patients with bladder cancer (ill), such as polypeptide markers No. 1 to 44, but absent or only rarely present in subjects with no bladder cancer (control). In addition, there are polypeptide markers which are present in subjects with no bladder cancer, but are less frequently or not at all present in subjects with bladder cancer, for example, Nos. 45 to 149 (Table 2).

A frequency marker is a variant of an amplitude marker in which the amplitude is low in some samples. It is possible to convert such frequency markers to amplitude markers by including the corresponding samples in which the marker is not found into the calculation of the amplitude with a very small amplitude, on the order of the detection limit.

TABLE 2
Polypeptide markers (frequency markers) for the diagnosis
of bladder cancer, their molecular masses and migration times
as well as their presence and absence (occurrence) in groups
of patients suffering from bladder cancer (BC) and control groups
as a factor (1 = 100%, 0 = 0%; sample processing and
measurement as described in the Example).

| | Occurrence in | Occurrence in |
|---|---|---|
| Number | group BC | Control |
| 1 | 0.86 | 0.13 |
| 2 | 0.71 | 0 |
| 3 | 0.73 | 0.03 |
| 4 | 0.71 | 0.07 |
| 5 | 0.64 | 0.03 |
| 6 | 0.64 | 0.03 |
| 7 | 0.71 | 0.13 |
| 8 | 0.71 | 0.13 |
| 9 | 0.73 | 0.16 |
| 10 | 0.67 | 0.09 |
| 11 | 0.57 | 0 |
| 12 | 0.92 | 0.35 |
| 13 | 0.57 | 0 |
| 14 | 0.57 | 0 |
| 15 | 0.57 | 0.03 |
| 16 | 0.57 | 0.03 |
| 17 | 0.57 | 0.03 |
| 18 | 0.57 | 0.03 |
| 19 | 0.57 | 0.03 |
| 20 | 0.67 | 0.13 |
| 21 | 0.78 | 0.25 |
| 22 | 0.69 | 0.16 |
| 23 | 0.78 | 0.25 |
| 24 | 0.69 | 0.17 |
| 25 | 0.56 | 0.03 |
| 26 | 0.57 | 0.05 |
| 27 | 0.67 | 0.16 |
| 28 | 0.6 | 0.09 |
| 29 | 0.51 | 0 |
| 30 | 0.86 | 0.35 |
| 31 | 0.91 | 0.41 |
| 32 | 0.71 | 0.22 |
| 33 | 0.51 | 0.03 |
| 34 | 0.58 | 0.09 |
| 35 | 0.89 | 0.41 |
| 36 | 0.51 | 0.03 |
| 37 | 0.73 | 0.25 |
| 38 | 0.51 | 0.03 |
| 39 | 0.62 | 0.16 |
| 40 | 0.69 | 0.22 |
| 41 | 0.58 | 0.13 |
| 42 | 0.58 | 0.13 |
| 43 | 0.73 | 0.38 |
| 44 | 0.8 | 0.5 |
| 45 | 0.42 | 0.72 |
| 46 | 0.51 | 0.81 |
| 47 | 0.44 | 0.75 |
| 48 | 0.62 | 0.95 |
| 49 | 0.57 | 0.9 |
| 50 | 0.57 | 0.93 |
| 51 | 0.29 | 0.65 |
| 52 | 0.29 | 0.68 |
| 53 | 0.42 | 0.81 |
| 54 | 0.57 | 0.97 |
| 55 | 0.14 | 0.55 |
| 56 | 0.31 | 0.78 |
| 57 | 0.33 | 0.81 |
| 58 | 0 | 0.5 |
| 59 | 0 | 0.5 |
| 60 | 0 | 0.5 |
| 61 | 0 | 0.5 |
| 62 | 0 | 0.5 |
| 63 | 0 | 0.5 |
| 64 | 0 | 0.5 |
| 65 | 0 | 0.5 |
| 66 | 0.29 | 0.8 |
| 67 | 0.14 | 0.65 |
| 68 | 0.14 | 0.65 |
| 69 | 0.14 | 0.65 |
| 70 | 0.29 | 0.8 |
| 71 | 0.29 | 0.8 |
| 72 | 0.14 | 0.65 |
| 73 | 0.14 | 0.65 |
| 74 | 0.29 | 0.8 |
| 75 | 0.18 | 0.7 |
| 76 | 0.33 | 0.85 |
| 77 | 0.08 | 0.6 |
| 78 | 0.43 | 0.95 |
| 79 | 0.43 | 0.95 |
| 80 | 0.31 | 0.82 |
| 81 | 0.2 | 0.72 |
| 82 | 0.14 | 0.68 |
| 83 | 0.14 | 0.68 |
| 84 | 0.14 | 0.68 |
| 85 | 0.14 | 0.68 |
| 86 | 0 | 0.53 |
| 87 | 0 | 0.53 |
| 88 | 0.29 | 0.82 |
| 89 | 0.29 | 0.82 |
| 90 | 0.29 | 0.82 |
| 91 | 0.29 | 0.82 |
| 92 | 0.29 | 0.82 |
| 93 | 0.29 | 0.82 |
| 94 | 0.29 | 0.82 |
| 95 | 0.26 | 0.8 |
| 96 | 0.43 | 0.97 |
| 97 | 0.43 | 0.97 |
| 98 | 0.43 | 0.97 |
| 99 | 0.29 | 0.85 |
| 100 | 0.14 | 0.7 |
| 101 | 0.29 | 0.85 |
| 102 | 0.14 | 0.7 |
| 103 | 0.29 | 0.85 |
| 104 | 0.29 | 0.85 |
| 105 | 0.29 | 0.85 |
| 106 | 0.26 | 0.82 |
| 107 | 0 | 0.57 |
| 108 | 0.26 | 0.82 |
| 109 | 0 | 0.57 |
| 110 | 0 | 0.57 |
| 111 | 0 | 0.57 |
| 112 | 0 | 0.57 |
| 113 | 0.43 | 1 |
| 114 | 0.24 | 0.81 |
| 115 | 0.14 | 0.72 |
| 116 | 0.14 | 0.72 |
| 117 | 0.14 | 0.72 |
| 118 | 0.29 | 0.88 |
| 119 | 0.29 | 0.88 |
| 120 | 0.29 | 0.88 |
| 121 | 0.36 | 0.95 |
| 122 | 0 | 0.6 |
| 123 | 0 | 0.6 |
| 124 | 0 | 0.6 |
| 125 | 0 | 0.6 |
| 126 | 0.29 | 0.9 |
| 127 | 0.14 | 0.75 |
| 128 | 0.29 | 0.9 |
| 129 | 0.18 | 0.8 |
| 130 | 0.13 | 0.75 |
| 131 | 0.31 | 0.93 |
| 132 | 0.14 | 0.78 |
| 133 | 0 | 0.63 |
| 134 | 0.28 | 0.93 |
| 135 | 0 | 0.65 |
| 136 | 0.18 | 0.82 |
| 137 | 0.14 | 0.8 |
| 138 | 0.14 | 0.8 |
| 139 | 0 | 0.68 |
| 140 | 0 | 0.68 |
| 141 | 0 | 0.7 |
| 142 | 0 | 0.7 |
| 143 | 0 | 0.75 |
| 144 | 0 | 0.75 |
| 145 | 0.14 | 0.95 |
| 146 | 0.15 | 0.55 |
| 147 | 0.15 | 0.6 |
| 148 | 0.15 | 0.57 |
| 149 | 0.15 | 0.56 |

The use of markers 1-146 is particularly preferred.

In addition or also alternatively to the frequency markers (determination of presence or absence), the amplitude markers as stated in Tables 3 and 4 may also be used for the diagnosis of bladder cancer (Nos. 150-836). Amplitude markers are used in such a way that the presence or absence is not critical, but the height of the signal (the amplitude) decides if the signal is present in both groups. In Tables 3 and 4, the mean normalized amplitudes of the corresponding signals (characterized by mass and migration time) averaged over all samples measured are stated. Two normalization methods are possible to achieve comparability between differently concentrated samples or different measuring methods.

In the first approach, all peptide signals of a sample are normalized to a total amplitude of 1 million counts. Therefore, the respective mean amplitudes of the individual markers are stated as parts per million (ppm). The amplitude markers obtained by this method are shown in Table 3 (Nos. 150-185).

In addition, it is possible to define further amplitude markers by an alternative normalization method: In this case, all peptide signals of one sample are scaled with a common normalization factor. Thus, a linear regression is formed between the peptide amplitudes of the individual samples and the reference values of all known polypeptides. The slope of the regression line just corresponds to the relative concentration and is used as a normalization factor for this sample. The biomarkers obtained by this normalization method are shown in Table 4 (Nos. 186-836).

The use of markers 150-185 is particularly preferred.

All groups employed consist of at least 40 individual patient or control samples in order to obtain a reliable mean amplitude. The decision for a diagnosis (bladder cancer or not) is made as a function of how high the amplitude of the respective polypeptide markers in the patient sample is in comparison with the mean amplitudes in the control groups or the bladder cancer group. If the amplitude rather corresponds to the mean amplitudes of the bladder cancer group, the existence of bladder cancer is to be considered, and if it rather corresponds to the mean amplitudes of the control group, the non-existence of bladder cancer is to be considered. The distance between the measured value and the mean amplitude can be considered a probability of the sample's belonging to a certain group.

A more exact definition for the procedure according to Approach 1 shall be given by means of marker No. 162 (Table 3). The mean amplitude of the marker is significantly increased in bladder cancer (6370 ppm vs. 1431 ppm in the control group). Now, if the value for this marker in a patient sample is from 0 to 1431 ppm or exceeds this range by a maximum of 20%, i.e., from 0 to 1717 ppm, then this sample belongs to the control group. If the value is 6370 ppm or up to 20% below, or higher, i.e., between 5096 and very high values, the existence of bladder cancer is to be considered.

For Approach 2, an illustrative explanation for the procedure may be given by means of marker No. 192 (Table 4). The mean amplitude of the marker is significantly increased in bladder cancer (374.91 counts vs. 44.29 counts in the control group). Now, if the value for this marker is from 0 to 44.29 counts in a sample from a patient or exceeds this range by a maximum of 20%, i.e., from 0 to 53.15 counts, then this sample belongs to the control group. If the value is 374.91 counts or up to 20% below, or higher, i.e., between 300 counts and very high values, the existence of bladder cancer is to be considered. Alternatively, the distance between the measured value and the mean amplitude may be considered a probability of the sample's belonging to a certain group.

TABLE 3
Amplitude markers from ppm normalization (Approach 1)

| | | Mean amplitude in |
|---|---|---|
| | Mean amplitude in | control samples |
| Number | group BC [ppm] | [ppm] |
| 150 | 512 | 234 |
| 151 | 4341 | 11833 |
| 152 | 320 | 107 |
| 153 | 4943 | 19582 |
| 154 | 831 | 404 |
| 155 | 1615 | 4183 |
| 156 | 882 | 2816 |
| 157 | 532 | 573 |
| 158 | 922 | 2703 |
| 159 | 3070 | 7231 |
| 160 | 1863 | 3755 |
| 161 | 2322 | 5187 |
| 162 | 6370 | 1431 |
| 163 | 1394 | 580 |
| 164 | 1039 | 428 |
| 165 | 225 | 542 |
| 166 | 769 | 1578 |
| 167 | 42329 | 112162 |
| 168 | 2602 | 981 |
| 169 | 321 | 745 |
| 170 | 106 | 308 |
| 171 | 596 | 1337 |
| 172 | 4128 | 10960 |
| 173 | 761 | 1961 |
| 174 | 200 | 428 |
| 175 | 2150 | 4372 |
| 176 | 995 | 2026 |
| 177 | 215 | 654 |
| 178 | 215 | 449 |
| 179 | 281 | 574 |
| 180 | 691 | 1578 |
| 181 | 1860 | 4502 |
| 182 | 1078 | 6650 |
| 183 | 406 | 846 |
| 184 | 342 | 1360 |
| 185 | 1425 | 10166 |

TABLE 4
Amplitude markers from normalization with normalization
factor (Approach 2)

| | Mean | Mean |
|---|---|---|
| | amplitude | amplitude |
| | in group | in |
| Number | BC | Controls |
| 186 | 119.63 | 80.79 |
| 187 | 77.4 | 56.61 |
| 188 | 44.83 | 19.49 |
| 189 | 75.44 | 45.2 |
| 190 | 118.21 | 235.42 |
| 191 | 74.62 | 33.56 |
| 192 | 374.91 | 44.29 |
| 193 | 157.08 | 123.78 |
| 194 | 453.57 | 10.31 |
| 195 | 88 | 45.2 |
| 196 | 90.46 | 720.98 |
| 197 | 111.95 | 85.68 |
| 198 | 410.43 | 469.76 |
| 199 | 427.05 | 319.41 |
| 200 | 83.12 | 93.94 |
| 201 | 102.07 | 57.89 |
| 202 | 238.89 | 119.03 |
| 203 | 111.31 | 90.37 |
| 204 | 195.66 | 197.97 |
| 205 | 223.5 | 223.37 |
| 206 | 149.65 | 149.75 |
| 207 | 159.86 | 56.01 |
| 208 | 353.83 | 70.68 |
| 209 | 79.65 | 40.47 |
| 210 | 669.1 | 617.85 |
| 211 | 153.61 | 135.8 |
| 212 | 163.45 | 287.56 |
| 213 | 1141.91 | 27.72 |
| 214 | 216.39 | 127.03 |
| 215 | 833.58 | 39.12 |
| 216 | 139 | 88.24 |
| 217 | 397.21 | 292.45 |
| 218 | 126.14 | 93.76 |
| 219 | 421.86 | 309.18 |
| 220 | 136.07 | 221.65 |
| 221 | 152.19 | 120.89 |
| 222 | 219.55 | 145.69 |
| 223 | 125.21 | 31.02 |
| 224 | 84.53 | 308.11 |
| 225 | 83.05 | 172.95 |
| 226 | 1842.39 | 1141.1 |
| 227 | 1324.02 | 1031.5 |
| 228 | 350.65 | 89.47 |
| 229 | 170.43 | 64.39 |
| 230 | 190.08 | 78.16 |
| 231 | 489.23 | 26.8 |
| 232 | 1996.47 | 72.63 |
| 233 | 188.24 | 95.18 |
| 234 | 161.17 | 119.99 |
| 235 | 103.9 | 97.75 |
| 236 | 480.36 | 237.07 |
| 237 | 5707.2 | 7336.8 |
| 238 | 379.06 | 56.43 |
| 239 | 308.98 | 301.08 |
| 240 | 462.23 | 78.2 |
| 241 | 194.93 | 151.59 |
| 242 | 5055.93 | 4175.1 |
| 243 | 366.14 | 189.83 |
| 244 | 367.44 | 170.6 |
| 245 | 224.51 | 141.51 |
| 246 | 183.13 | 124.78 |
| 247 | 106.82 | 88.51 |
| 248 | 3078.26 | 140.75 |
| 249 | 3190.15 | 3178.22 |
| 250 | 368.81 | 72.76 |
| 251 | 256.09 | 245.38 |
| 252 | 130.05 | 134.05 |
| 253 | 448.49 | 239.5 |
| 254 | 232.48 | 315.01 |
| 255 | 205.39 | 158.14 |
| 256 | 404.85 | 342.62 |
| 257 | 249.07 | 176.66 |
| 258 | 94.05 | 45.1 |
| 259 | 175.28 | 119.61 |
| 260 | 202.45 | 124.75 |
| 261 | 853.59 | 1140.19 |
| 262 | 1327.35 | 1993.54 |
| 263 | 356.82 | 284.59 |
| 264 | 265.05 | 150.92 |
| 265 | 558.75 | 451.29 |
| 266 | 221.77 | 143.72 |
| 267 | 1128.53 | 72.92 |
| 268 | 331.84 | 200.42 |
| 269 | 505.65 | 421.74 |
| 270 | 261.75 | 225.98 |
| 271 | 169.57 | 130.21 |
| 272 | 266.62 | 223.68 |
| 273 | 1362.12 | 1707.78 |
| 274 | 294.36 | 402.59 |
| 275 | 221.55 | 166.84 |
| 276 | 335.58 | 44.14 |
| 277 | 259.95 | 223.47 |
| 278 | 768.74 | 684.34 |
| 279 | 251.7 | 214.42 |
| 280 | 169.01 | 89.8 |
| 281 | 206.44 | 145.14 |
| 282 | 3791.23 | 83.4 |
| 283 | 147.66 | 91.52 |
| 284 | 257.97 | 474.54 |
| 285 | 2312.84 | 72.28 |
| 286 | 2355.55 | 150.4 |
| 287 | 1856.18 | 0 |
| 288 | 22744.1 | 27845.6 |
| 289 | 579.96 | 1024.9 |
| 290 | 181 | 79.4 |
| 291 | 449.65 | 287.08 |
| 292 | 8039.6 | 6153.2 |
| 293 | 400.81 | 327.89 |
| 294 | 310.29 | 151 |
| 295 | 1031.54 | 860.46 |
| 296 | 633.16 | 564.48 |
| 297 | 218.71 | 298.42 |
| 298 | 687.69 | 58.53 |
| 299 | 462.1 | 597.81 |
| 300 | 1581.02 | 1215.02 |
| 301 | 2630.29 | 3679.53 |
| 302 | 979.29 | 1913.27 |
| 303 | 243.92 | 96.75 |
| 304 | 173.39 | 55.41 |
| 305 | 148.48 | 73.15 |
| 306 | 156.84 | 179.86 |
| 307 | 469.03 | 595.19 |
| 308 | 133.11 | 102.43 |
| 309 | 792.94 | 497.12 |
| 310 | 1537.92 | 2939.46 |
| 311 | 1580.14 | 1795.44 |
| 312 | 193.48 | 190.55 |
| 313 | 794.93 | 201.72 |
| 314 | 928.42 | 699.24 |
| 315 | 143.26 | 115.01 |
| 316 | 303.03 | 129.16 |
| 317 | 1342.16 | 932.91 |
| 318 | 1790.3 | 3379.16 |
| 319 | 746.14 | 204.66 |
| 320 | 706.29 | 396.08 |
| 321 | 609.39 | 342.54 |
| 322 | 201.07 | 279.53 |
| 323 | 440.59 | 252.99 |
| 324 | 1500.12 | 166.99 |
| 325 | 231.13 | 194.08 |
| 326 | 690.08 | 362.46 |
| 327 | 239.27 | 204.76 |
| 328 | 312.93 | 156.8 |
| 329 | 325.22 | 238 |
| 330 | 8160.78 | 334.35 |
| 331 | 271 | 198.75 |
| 332 | 543.53 | 962.52 |
| 333 | 10101.8 | 12695.3 |
| 334 | 459.58 | 129.16 |
| 335 | 648.02 | 415.43 |
| 336 | 163.79 | 121.63 |
| 337 | 205.35 | 258.72 |
| 338 | 4607.6 | 550.93 |
| 339 | 112.46 | 181.81 |
| 340 | 1168.83 | 221.78 |
| 341 | 407.83 | 425.82 |
| 342 | 429.65 | 329.69 |
| 343 | 1760.83 | 2575.03 |
| 344 | 514.01 | 309.71 |
| 345 | 2515.14 | 115.68 |
| 346 | 565.18 | 631.08 |
| 347 | 348.79 | 312.62 |
| 348 | 184.84 | 126.88 |
| 349 | 1992.63 | 2743.26 |
| 350 | 4620.61 | 341.71 |
| 351 | 21183.3 | 33275.8 |
| 352 | 3541.13 | 4832.9 |
| 353 | 267.96 | 178.61 |
| 354 | 260.72 | 600.85 |
| 355 | 598.13 | 660.69 |
| 356 | 318.88 | 381.49 |
| 357 | 784.39 | 610.28 |
| 358 | 394.52 | 233.81 |
| 359 | 2575.84 | 1071.44 |
| 360 | 2460.15 | 3145.77 |
| 361 | 1463.89 | 405.51 |
| 362 | 330.7 | 296.81 |
| 363 | 430 | 232.16 |
| 364 | 550.02 | 691.79 |
| 365 | 1968.51 | 197.72 |
| 366 | 350.92 | 381.91 |
| 367 | 706.87 | 835.85 |
| 368 | 2384.33 | 175.16 |
| 369 | 3854.98 | 5258.01 |
| 370 | 14948.2 | 21376.3 |
| 371 | 728.16 | 250.02 |
| 372 | 552.5 | 144.36 |
| 373 | 210.05 | 246.6 |
| 374 | 347.41 | 249.22 |
| 375 | 207.87 | 315.21 |
| 376 | 357.91 | 878.46 |
| 377 | 274.83 | 200.69 |
| 378 | 852.25 | 552.92 |
| 379 | 427.78 | 628.41 |
| 380 | 344.12 | 480.68 |
| 381 | 10371.5 | 253.26 |
| 382 | 1974.82 | 2562.08 |
| 383 | 869.39 | 408.55 |
| 384 | 72.76 | 94.63 |
| 385 | 441.96 | 297.83 |
| 386 | 401.89 | 356.62 |
| 387 | 1259.94 | 1584.04 |
| 388 | 1178.53 | 2616.54 |
| 389 | 358.67 | 507.39 |
| 390 | 482.45 | 537.75 |
| 391 | 1542.53 | 2790.5 |
| 392 | 503.57 | 330.15 |
| 393 | 2430.83 | 3353.77 |
| 394 | 523.9 | 843.44 |
| 395 | 478.99 | 596.56 |
| 396 | 1207.17 | 1548.95 |
| 397 | 248.65 | 483.71 |
| 398 | 166.63 | 140.29 |
| 399 | 1049.78 | 696.02 |
| 400 | 3862.62 | 4198.48 |
| 401 | 733.5 | 999.31 |
| 402 | 5193.79 | 7951.13 |
| 403 | 2139.76 | 2730.17 |
| 404 | 916.36 | 816.75 |
| 405 | 1684.49 | 1580.08 |
| 406 | 237.78 | 343.53 |
| 407 | 2026.66 | 464.44 |
| 408 | 196.63 | 264.47 |
| 409 | 304.49 | 439.85 |
| 410 | 337.51 | 477.51 |
| 411 | 255.82 | 416.12 |
| 412 | 1221.53 | 1619.64 |
| 413 | 9331.13 | 14100.7 |
| 414 | 197.33 | 300.63 |
| 415 | 212 | 165.73 |
| 416 | 1346.36 | 1460.35 |
| 417 | 820.34 | 1192.4 |
| 418 | 1548.36 | 1967.46 |
| 419 | 1218.82 | 1006.41 |
| 420 | 218.44 | 251.93 |
| 421 | 667.59 | 467.84 |
| 422 | 630.62 | 817.36 |
| 423 | 1128.79 | 1580.15 |
| 424 | 584.6 | 641.55 |
| 425 | 923.66 | 1154.89 |
| 426 | 245.2 | 209.04 |
| 427 | 208.86 | 122.78 |
| 428 | 2141.81 | 2794.73 |
| 429 | 2673.32 | 3482.39 |
| 430 | 194.01 | 349.03 |
| 431 | 244.32 | 190.7 |
| 432 | 171.13 | 269.09 |
| 433 | 389.06 | 220.01 |
| 434 | 950.01 | 1103.05 |
| 435 | 737.26 | 1145.11 |
| 436 | 698.31 | 1006.16 |
| 437 | 1034.44 | 1328.66 |
| 438 | 1549.89 | 2565.32 |
| 439 | 242.37 | 346.97 |
| 440 | 4225.33 | 7163.11 |
| 441 | 2026.98 | 2749.89 |
| 442 | 1669.33 | 64.04 |
| 443 | 453.3 | 412.54 |
| 444 | 284.09 | 110.58 |
| 445 | 452.02 | 464.12 |
| 446 | 10928.25 | 6009.48 |
| 447 | 248.66 | 244.14 |
| 448 | 544.89 | 495.75 |
| 449 | 91.29 | 220.69 |
| 450 | 690.87 | 378.52 |
| 451 | 372.26 | 469.99 |
| 452 | 695.43 | 437.27 |
| 453 | 197.46 | 344.87 |
| 454 | 636.99 | 851.23 |
| 455 | 109.04 | 132.39 |
| 456 | 114.34 | 159.23 |
| 457 | 247.87 | 278.24 |
| 458 | 1565.61 | 2420.43 |
| 459 | 2149.51 | 2722.27 |
| 460 | 1761.85 | 1313.47 |
| 461 | 458.34 | 487.39 |
| 462 | 1518.75 | 1405.34 |
| 463 | 339.47 | 310.42 |
| 464 | 9070.76 | 1838.11 |
| 465 | 3861.14 | 2831.77 |
| 466 | 423.42 | 203.85 |
| 467 | 283.34 | 170.89 |
| 468 | 367.72 | 315.21 |
| 469 | 1424.84 | 339.86 |
| 470 | 1243.61 | 366.74 |
| 471 | 519.88 | 332.05 |
| 472 | 457.11 | 528.19 |
| 473 | 1906.66 | 2592.95 |
| 474 | 840.95 | 1804.57 |
| 475 | 285.04 | 113.11 |
| 476 | 232.38 | 117.03 |
| 477 | 4444.41 | 11160.44 |
| 478 | 698.61 | 390.57 |
| 479 | 648.4 | 507.64 |
| 480 | 442.16 | 240.49 |
| 481 | 16250.01 | 1560.5 |
| 482 | 945.07 | 559.31 |
| 483 | 1339.21 | 1514.58 |
| 484 | 358.02 | 192.21 |
| 485 | 232.09 | 187.49 |
| 486 | 684.31 | 479.07 |
| 487 | 728.45 | 407.72 |
| 488 | 196.89 | 476.3 |
| 489 | 633.98 | 149.83 |
| 490 | 91.61 | 147.08 |
| 491 | 502.32 | 282.04 |
| 492 | 441.16 | 507.47 |
| 493 | 714.01 | 210.4 |
| 494 | 349.59 | 153.45 |
| 495 | 269.05 | 450.84 |
| 496 | 403.2 | 515.62 |
| 497 | 513.8 | 133.24 |
| 498 | 295.46 | 431.46 |
| 499 | 790.03 | 915.09 |
| 500 | 411.21 | 61.58 |
| 501 | 1600.21 | 707.59 |
| 502 | 185.12 | 169.93 |
| 503 | 211.53 | 252.44 |
| 504 | 202.48 | 131.9 |
| 505 | 106.45 | 139.9 |
| 506 | 172.8 | 123.01 |
| 507 | 2238.19 | 1347.32 |
| 508 | 4881.51 | 210.22 |
| 509 | 386.33 | 241.64 |
| 510 | 389.85 | 197.63 |
| 511 | 1044.25 | 1860.09 |
| 512 | 911.8 | 213.78 |
| 513 | 165.39 | 235.62 |
| 514 | 366.71 | 78.89 |
| 515 | 3952.23 | 943.15 |
| 516 | 394.1 | 154.31 |
| 517 | 721.07 | 344.83 |
| 518 | 866.53 | 120.51 |
| 519 | 384.56 | 159.41 |
| 520 | 1491.26 | 589.3 |
| 521 | 969.57 | 410.04 |
| 522 | 1522.66 | 1334.42 |
| 523 | 145.86 | 141.48 |
| 524 | 189.68 | 287.18 |
| 525 | 1514.05 | 111.26 |
| 526 | 270.67 | 55.86 |
| 527 | 223.41 | 313.99 |
| 528 | 220.43 | 162.9 |
| 529 | 775.4 | 654.44 |
| 530 | 115.96 | 136.71 |
| 531 | 2682.19 | 2753.18 |
| 532 | 1004.59 | 725.68 |
| 533 | 24215.6 | 23635.89 |
| 534 | 1380.23 | 1722.54 |
| 535 | 635.5 | 466.98 |
| 536 | 590.85 | 530.1 |
| 537 | 205.17 | 252.33 |
| 538 | 262.07 | 133.39 |
| 539 | 236.94 | 716.14 |
| 540 | 116.27 | 110.6 |
| 541 | 286.2 | 368.78 |
| 542 | 355.35 | 522.74 |
| 543 | 3635.8 | 755.11 |
| 544 | 282.24 | 163.79 |
| 545 | 258.35 | 676.03 |
| 546 | 336.29 | 179.19 |
| 547 | 411.08 | 341.22 |
| 548 | 288.6 | 126.04 |
| 549 | 433.38 | 207.91 |
| 550 | 468.04 | 764.16 |
| 551 | 514.82 | 290.72 |
| 552 | 309.49 | 252.04 |
| 553 | 352.9 | 63.73 |
| 554 | 832.83 | 559.35 |
| 555 | 213.46 | 131.69 |
| 556 | 2691.55 | 211.72 |
| 557 | 5649.48 | 1095.48 |
| 558 | 267.74 | 274.93 |
| 559 | 197.06 | 351.22 |
| 560 | 322.71 | 181.52 |
| 561 | 1774.17 | 965.1 |
| 562 | 674.15 | 1019.16 |
| 563 | 514.89 | 202.92 |
| 564 | 280.12 | 231.27 |
| 565 | 612.61 | 484.55 |
| 566 | 305.8 | 281.04 |
| 567 | 6126.15 | 6394.03 |
| 568 | 564.34 | 127.81 |
| 569 | 895.57 | 671.47 |
| 570 | 234.83 | 74.14 |
| 571 | 918.01 | 306.21 |
| 572 | 490.15 | 215.39 |
| 573 | 340.36 | 561.18 |
| 574 | 293.37 | 165.07 |
| 575 | 1457.87 | 1267 |
| 576 | 186.86 | 193.04 |
| 577 | 15200.82 | 840.95 |
| 578 | 445 | 510.51 |
| 579 | 503.8 | 219.86 |
| 580 | 3363.04 | 542.25 |
| 581 | 399.54 | 283.96 |
| 582 | 1073.6 | 416.48 |
| 583 | 344.17 | 359.53 |
| 584 | 679.11 | 618.79 |
| 585 | 657.73 | 217.1 |
| 586 | 3920.85 | 58.99 |
| 587 | 192.77 | 218.19 |
| 588 | 417.28 | 103.5 |
| 589 | 331.42 | 405.5 |
| 590 | 2854 | 126.77 |
| 591 | 809.25 | 1010.64 |
| 592 | 262.72 | 539.84 |
| 593 | 146.88 | 411.88 |
| 594 | 8079.35 | 1105.07 |
| 595 | 488.35 | 132.06 |
| 596 | 3335 | 859.68 |
| 597 | 516.5 | 679.53 |
| 598 | 1181.54 | 168.71 |
| 599 | 411.12 | 893.15 |
| 600 | 297.54 | 266.17 |
| 601 | 1122.23 | 1242.63 |
| 602 | 140.12 | 176.5 |
| 603 | 2821 | 1369.9 |
| 604 | 894.97 | 169.59 |
| 605 | 342.43 | 207.05 |
| 606 | 5208.16 | 8436.04 |
| 607 | 109.36 | 126.02 |
| 608 | 5005.47 | 886.26 |
| 609 | 385.8 | 404.35 |
| 610 | 384.35 | 2017.29 |
| 611 | 1447.69 | 90.16 |
| 612 | 189.43 | 158.12 |
| 613 | 886.48 | 201.67 |
| 614 | 171.69 | 342.56 |
| 615 | 712.07 | 723.54 |
| 616 | 604.67 | 979.18 |
| 617 | 645.45 | 1001.2 |
| 618 | 367.72 | 477.68 |
| 619 | 188.76 | 457.6 |
| 620 | 612.89 | 94.01 |
| 621 | 225.78 | 223.31 |
| 622 | 3910.57 | 6523.87 |
| 623 | 258.4 | 256.33 |
| 624 | 693.23 | 913.29 |
| 625 | 92.22 | 168.82 |
| 626 | 338.07 | 497.04 |
| 627 | 1743.21 | 858.44 |
| 628 | 1934.06 | 274.04 |
| 629 | 3254.92 | 22.36 |
| 630 | 173.04 | 158.48 |
| 631 | 375.54 | 200.28 |
| 632 | 317.73 | 93 |
| 633 | 2739.3 | 1120.66 |
| 634 | 563.71 | 786.49 |
| 635 | 1199.43 | 277.21 |
| 636 | 744.12 | 919.2 |
| 637 | 346.57 | 197.87 |
| 638 | 1090.93 | 283.03 |
| 639 | 290.46 | 140.43 |
| 640 | 111.85 | 66.25 |
| 641 | 455.21 | 420.21 |
| 642 | 345.83 | 199.62 |
| 643 | 1651.16 | 1011.11 |
| 644 | 4741.83 | 614.65 |
| 645 | 697.29 | 334.7 |
| 646 | 277.24 | 288.32 |
| 647 | 4690.58 | 3358.31 |
| 648 | 240.79 | 294.91 |
| 649 | 641.99 | 200.78 |
| 650 | 371.35 | 238.75 |
| 651 | 2349.83 | 1291.14 |
| 652 | 193.38 | 143.66 |
| 653 | 1226.05 | 1935.03 |
| 654 | 5138.39 | 3725.55 |
| 655 | 3230.3 | 7444.98 |
| 656 | 746.95 | 386.71 |
| 657 | 239.61 | 201.28 |
| 658 | 464.77 | 869.33 |
| 659 | 210.32 | 980.59 |
| 660 | 3851.92 | 329.08 |
| 661 | 2485.76 | 2717 |
| 662 | 347.83 | 623.37 |
| 663 | 145.28 | 189.73 |
| 664 | 479.37 | 1413.9 |
| 665 | 690.96 | 407.33 |
| 666 | 440.32 | 770.9 |
| 667 | 348.99 | 416.59 |
| 668 | 321.33 | 612.28 |
| 669 | 330.69 | 333.64 |
| 670 | 465.29 | 768.38 |
| 671 | 804.23 | 897.03 |
| 672 | 331.26 | 117.39 |
| 673 | 18864.8 | 34.43 |
| 674 | 222.74 | 256.29 |
| 675 | 777.58 | 927.95 |
| 676 | 218.75 | 161.04 |
| 677 | 675.28 | 307.55 |
| 678 | 1427.9 | 1826.7 |
| 679 | 1500.77 | 1729 |
| 680 | 1179.77 | 1493.4 |
| 681 | 1035.25 | 621.15 |
| 682 | 827.45 | 539.76 |
| 683 | 928.3 | 504.81 |
| 684 | 379.72 | 200.01 |
| 685 | 454.16 | 274.58 |
| 686 | 1247.87 | 797.98 |
| 687 | 21125.9 | 178.01 |
| 688 | 131 | 156.14 |
| 689 | 86.47 | 120.23 |
| 690 | 184.62 | 196.27 |
| 691 | 326.21 | 74.96 |
| 692 | 467.89 | 307.96 |
| 693 | 796.96 | 485.58 |
| 694 | 2248.11 | 1306 |
| 695 | 492.82 | 227.88 |
| 696 | 443.67 | 725.02 |
| 697 | 810.12 | 535.52 |
| 698 | 998.77 | 1170 |
| 699 | 192.96 | 140.39 |
| 700 | 115 | 132.51 |
| 701 | 2241.77 | 2710.4 |
| 702 | 770.19 | 988.21 |
| 703 | 401.99 | 247.99 |
| 704 | 360.05 | 552.79 |
| 705 | 107.75 | 98.14 |
| 706 | 998.83 | 1032.7 |
| 707 | 164.07 | 209.94 |
| 708 | 1627.4 | 1118.7 |
| 709 | 763.49 | 250.07 |
| 710 | 773.85 | 675.53 |
| 711 | 155.22 | 286.79 |
| 712 | 1272.34 | 2512.1 |
| 713 | 27791.3 | 1191.1 |
| 714 | 459.25 | 281.17 |
| 715 | 941.28 | 1159 |
| 716 | 341.85 | 213.77 |
| 717 | 869.93 | 987.76 |
| 718 | 35304.9 | 419.08 |
| 719 | 295.28 | 457.92 |
| 720 | 97.18 | 91.86 |
| 721 | 5238.18 | 742.94 |
| 722 | 1105.15 | 2446.05 |
| 723 | 15994.6 | 18395.9 |
| 724 | 25574.2 | 56.87 |
| 725 | 223.22 | 334.29 |
| 726 | 390.33 | 591.38 |
| 727 | 874.08 | 1016.73 |
| 728 | 129.08 | 109.04 |
| 729 | 287.17 | 386.91 |
| 730 | 938.84 | 1689.36 |
| 731 | 249.13 | 373.29 |
| 732 | 2681.59 | 934.95 |
| 733 | 518.6 | 570.92 |
| 734 | 304.49 | 410.25 |
| 735 | 515.18 | 321.95 |
| 736 | 719.9 | 389.6 |
| 737 | 162.63 | 213.43 |
| 738 | 202.87 | 327.64 |
| 739 | 310.7 | 366.38 |
| 740 | 1057.06 | 967.69 |
| 741 | 1305.72 | 1525.5 |
| 742 | 406.27 | 251.43 |
| 743 | 209.98 | 332.04 |
| 744 | 779.02 | 507.03 |
| 745 | 509.21 | 780.22 |
| 746 | 550.52 | 205.81 |
| 747 | 303.67 | 178.71 |
| 748 | 391.6 | 345.85 |
| 749 | 161.04 | 486.26 |
| 750 | 1633.21 | 1883.97 |
| 751 | 1948.88 | 819.34 |
| 752 | 947.01 | 1410.71 |
| 753 | 88.29 | 103.94 |
| 754 | 748.98 | 398.24 |
| 755 | 5654.78 | 2358.14 |
| 756 | 176.88 | 284.44 |
| 757 | 641.35 | 134.74 |
| 758 | 193.83 | 250.67 |
| 759 | 265.87 | 443.61 |
| 760 | 1611.2 | 2134.8 |
| 761 | 895 | 953.08 |
| 762 | 4307.33 | 1318.11 |
| 763 | 425.51 | 320.2 |
| 764 | 157.27 | 248.54 |
| 765 | 2159.53 | 4308.89 |
| 766 | 601.24 | 540.73 |
| 767 | 259.57 | 190.8 |
| 768 | 268.04 | 788.11 |
| 769 | 1124.91 | 947.92 |
| 770 | 345.81 | 258.56 |
| 771 | 4076.84 | 585.19 |
| 772 | 1545.75 | 627.71 |
| 773 | 635.06 | 927.49 |
| 774 | 1015.92 | 550.41 |
| 775 | 475.86 | 490.03 |
| 776 | 5071.3 | 7395.4 |
| 777 | 271.15 | 292.43 |
| 778 | 502.9 | 356.61 |
| 779 | 4550.74 | 733.37 |
| 780 | 811.84 | 153.69 |
| 781 | 5268.58 | 6177 |
| 782 | 964.7 | 441.9 |
| 783 | 1288.41 | 1241.8 |
| 784 | 761.9 | 106.33 |
| 785 | 302.57 | 220.29 |
| 786 | 441.39 | 34.69 |
| 787 | 4805.19 | 408.09 |
| 788 | 724.85 | 750.37 |
| 789 | 2797.11 | 4381.9 |
| 790 | 1660.28 | 627.22 |
| 791 | 82.16 | 51.86 |
| 792 | 1500.15 | 777.08 |
| 793 | 131.83 | 184.79 |
| 794 | 1950.66 | 1314.3 |
| 795 | 445.08 | 140.52 |
| 796 | 421.99 | 182.5 |
| 797 | 426.27 | 390.57 |
| 798 | 498.29 | 564.91 |
| 799 | 483.58 | 288.48 |
| 800 | 177.85 | 148.57 |
| 801 | 1927.19 | 290.82 |
| 802 | 1908.71 | 378.98 |
| 803 | 1356.99 | 309.29 |
| 804 | 590.4 | 454.65 |
| 805 | 479.33 | 263.09 |
| 806 | 1688.17 | 334.94 |
| 807 | 2524.15 | 1194 |
| 808 | 613.82 | 295.85 |
| 809 | 1847.5 | 1489 |
| 810 | 551.98 | 311.34 |
| 811 | 908.48 | 388.74 |
| 812 | 124.16 | 66.69 |
| 813 | 1567.12 | 859.71 |
| 814 | 537.13 | 331.57 |
| 815 | 1081.92 | 743.86 |
| 816 | 1288.32 | 753.52 |
| 817 | 1522.33 | 1216.1 |
| 818 | 3743.12 | 844.55 |
| 819 | 211.47 | 155.57 |
| 820 | 232.71 | 324.57 |
| 821 | 311.5 | 454.92 |
| 822 | 433.81 | 689.88 |
| 823 | 2674.9 | 1470.8 |
| 824 | 2022.24 | 1507.3 |
| 825 | 4346.69 | 2618.9 |
| 826 | 2344.92 | 1552.8 |
| 827 | 898.51 | 575.57 |
| 828 | 901.65 | 505.99 |
| 829 | 1946.95 | 9898.6 |
| 830 | 961.96 | 4337.1 |
| 831 | 1561.13 | 2095.7 |
| 832 | 5309.3 | 1583.6 |
| 833 | 532.67 | 313.23 |
| 834 | 855.38 | 600.48 |
| 835 | 900.1 | 189.35 |
| 836 | 4098.86 | 2173.05 |

The polypeptide markers are also suitable for determining a tumor stage. For the differential diagnosis between patients with no BC and patients with superficial BC, the frequencies according to Table 5 and the amplitudes according to Table 6 are suitable.

TABLE 5
Frequency markers for distinction between
superficial BC and patients with no BC

| Number | Occurrence in group BC superficial | Occurrence in control |
|---|---|---|
| 146 | 0.13 | 0.55 |
| 147 | 0.15 | 0.6 |
| 148 | 0.17 | 0.57 |

TABLE 6
Amplitude markers for distinction between superficial BC
and patients with no BC

| | Mean | |
|---|---|---|
| | amplitude | Mean |
| | in group | amplitude |
| | BC | in |
| Number | superficial | controls |
| 7 | 116.31 | 171.88 |
| 8 | 3346.12 | 1718.08 |
| 20 | 1414.75 | 204.11 |
| 23 | 1152.72 | 758.3 |
| 31 | 13833.1 | 1997.34 |
| 37 | 404.73 | 159.1 |
| 40 | 451.06 | 159.59 |
| 43 | 203.42 | 135.13 |
| 44 | 619.98 | 943.28 |
| 58 | 1631.78 | 2420.43 |
| 61 | 109.9 | 106.43 |
| 63 | 399.18 | 318.87 |
| 70 | 255.14 | 168.72 |
| 71 | 505.84 | 220.23 |
| 81 | 1457.02 | 1083.72 |
| 89 | 287.82 | 410.5 |
| 90 | 387.02 | 395.23 |
| 92 | 971.64 | 1464.36 |
| 97 | 147.12 | 266.31 |
| 101 | 272.02 | 206.42 |
| 115 | 203.74 | 303.62 |
| 126 | 707.92 | 764.74 |
| 129 | 114.31 | 208.29 |
| 132 | 397.6 | 591.14 |
| 135 | 449.32 | 298.56 |
| 137 | 216.96 | 149.67 |
| 152 | 325.5 | 272.96 |
| 156 | 3756 | 2810.21 |
| 158 | 1109.21 | 1996.73 |
| 170 | 110.86 | 224.3 |
| 173 | 521.41 | 717.77 |
| 176 | 712.45 | 869.56 |
| 180 | 1377.28 | 2012.33 |
| 184 | 453.72 | 946.92 |
| 185 | 10671.27 | 12125.03 |
| 190 | 118.18 | 235.42 |
| 198 | 250.7 | 469.76 |
| 210 | 446.64 | 617.85 |
| 212 | 112.84 | 287.56 |
| 219 | 144 | 221.65 |
| 225 | 95.25 | 172.95 |
| 226 | 785.46 | 1141.14 |
| 237 | 4373.54 | 7336.84 |
| 239 | 273.08 | 301.08 |
| 249 | 2176.11 | 3178.22 |
| 261 | 1093.03 | 1140.19 |
| 262 | 1453.44 | 1993.54 |
| 266 | 82.68 | 143.72 |
| 269 | 272.05 | 421.74 |
| 271 | 115.76 | 130.21 |
| 272 | 133.52 | 223.68 |
| 275 | 195.93 | 402.59 |
| 284 | 155.65 | 474.54 |
| 288 | 18118.22 | 27845.62 |
| 289 | 705.44 | 1024.9 |
| 296 | 369.58 | 564.48 |
| 299 | 418.65 | 597.81 |
| 301 | 2355.61 | 3679.53 |
| 303 | 255.32 | 96.75 |
| 306 | 114.63 | 179.86 |
| 307 | 423.6 | 595.19 |
| 311 | 1632.87 | 1795.44 |
| 312 | 244.52 | 190.55 |
| 313 | 147.27 | 201.72 |
| 318 | 1130.23 | 3379.16 |
| 332 | 668.14 | 962.52 |
| 334 | 124.59 | 129.16 |
| 340 | 135.75 | 221.78 |
| 341 | 213.78 | 425.82 |
| 343 | 1942.45 | 2575.03 |
| 347 | 403.35 | 312.62 |
| 350 | 406.28 | 341.71 |
| 351 | 24046.12 | 33275.83 |
| 354 | 288.33 | 600.85 |
| 359 | 1679.91 | 1071.44 |
| 360 | 2442.99 | 3145.77 |
| 366 | 428.94 | 381.91 |
| 367 | 1048.83 | 835.85 |
| 371 | 737.87 | 250.02 |
| 373 | 158.25 | 246.6 |
| 374 | 416.91 | 249.22 |
| 375 | 200.86 | 315.21 |
| 376 | 431.93 | 878.46 |
| 381 | 3618.42 | 253.26 |
| 384 | 70.76 | 94.63 |
| 385 | 502.72 | 297.83 |
| 386 | 241.72 | 356.62 |
| 387 | 1388.15 | 1584.04 |
| 389 | 324.34 | 507.39 |
| 390 | 284.78 | 537.75 |
| 391 | 1787.19 | 2790.5 |
| 393 | 2600.66 | 3353.77 |
| 395 | 398.9 | 596.56 |
| 398 | 187.41 | 140.29 |
| 404 | 631.32 | 816.75 |
| 409 | 255.99 | 439.85 |
| 412 | 1364.55 | 1619.64 |
| 414 | 216.1 | 300.63 |
| 419 | 537.72 | 1006.41 |
| 423 | 1441.12 | 1580.15 |
| 430 | 171.47 | 349.03 |
| 432 | 194.68 | 269.09 |
| 435 | 713.74 | 1145.11 |
| 438 | 1960.48 | 2565.32 |
| 440 | 5360.16 | 7163.11 |
| 445 | 594.8 | 464.12 |
| 446 | 10467.64 | 6009.48 |
| 448 | 660.96 | 495.75 |
| 449 | 105.44 | 220.69 |
| 450 | 585.27 | 378.52 |
| 452 | 586.56 | 437.27 |
| 453 | 246.69 | 344.87 |
| 460 | 2511.6 | 1313.47 |
| 461 | 753.53 | 487.39 |
| 464 | 20601.57 | 1838.11 |
| 470 | 590.47 | 366.74 |
| 471 | 544.61 | 332.05 |
| 474 | 879.35 | 1804.57 |
| 477 | 5333.72 | 11160.44 |
| 478 | 665 | 390.57 |
| 480 | 182.45 | 240.49 |
| 481 | 30800.29 | 1560.5 |
| 482 | 1149.59 | 559.31 |
| 483 | 1217.27 | 1514.58 |
| 487 | 659.74 | 407.72 |
| 488 | 184.56 | 476.3 |
| 491 | 780.25 | 282.04 |
| 499 | 549.66 | 915.09 |
| 502 | 230.68 | 169.93 |
| 507 | 2145.46 | 1347.32 |
| 510 | 329.78 | 197.63 |
| 511 | 1149.81 | 1860.09 |
| 517 | 597.1 | 344.83 |
| 519 | 214.5 | 159.41 |
| 522 | 2040.96 | 1334.42 |
| 523 | 188.34 | 141.48 |
| 528 | 283.36 | 162.9 |
| 531 | 3574.56 | 2753.18 |
| 535 | 704.86 | 466.98 |
| 536 | 800.65 | 530.1 |
| 539 | 123.56 | 716.14 |
| 540 | 119.18 | 110.6 |
| 543 | 536.82 | 522.74 |
| 544 | 228.52 | 163.79 |
| 545 | 304.3 | 676.03 |
| 547 | 510.33 | 341.22 |
| 551 | 518.82 | 290.72 |
| 552 | 322.61 | 252.04 |
| 564 | 316.34 | 231.27 |
| 566 | 256.99 | 281.04 |
| 568 | 213.06 | 127.81 |
| 569 | 981 | 671.47 |
| 574 | 256.67 | 165.07 |
| 591 | 883.27 | 1010.64 |
| 595 | 204.98 | 132.06 |
| 599 | 349 | 893.15 |
| 600 | 410.22 | 266.17 |
| 601 | 1685.83 | 1242.63 |
| 602 | 130.05 | 176.5 |
| 605 | 458.37 | 207.05 |
| 609 | 409.71 | 404.35 |
| 610 | 621.93 | 2017.29 |
| 614 | 192.26 | 342.56 |
| 615 | 316.08 | 723.54 |
| 618 | 342.45 | 477.68 |
| 619 | 197.75 | 457.6 |
| 621 | 4385.63 | 6523.87 |
| 625 | 129.34 | 168.82 |
| 630 | 254.47 | 158.48 |
| 631 | 326.82 | 200.28 |
| 633 | 2257.95 | 1120.66 |
| 636 | 730.41 | 919.2 |
| 638 | 373.94 | 283.03 |
| 639 | 305.66 | 140.43 |
| 643 | 1759.51 | 1011.11 |
| 645 | 584.26 | 334.7 |
| 646 | 351.46 | 288.32 |
| 650 | 445.12 | 238.75 |
| 655 | 3310.08 | 7444.98 |
| 656 | 582.62 | 386.71 |
| 657 | 293.05 | 201.28 |
| 658 | 369.66 | 869.33 |
| 659 | 288.42 | 980.59 |
| 664 | 803.94 | 1413.92 |
| 665 | 587.16 | 407.33 |
| 666 | 469.6 | 770.9 |
| 668 | 454.53 | 612.28 |
| 670 | 421.48 | 333.64 |
| 676 | 284.62 | 161.04 |
| 681 | 891.35 | 621.15 |
| 682 | 1299.73 | 539.76 |
| 684 | 540.83 | 200.01 |
| 693 | 695.98 | 485.58 |
| 695 | 449.35 | 725.02 |
| 696 | 335.66 | 227.88 |
| 697 | 1024.07 | 535.52 |
| 699 | 141.11 | 140.39 |
| 704 | 195.53 | 552.79 |
| 705 | 132.97 | 98.14 |
| 706 | 659.99 | 1032.67 |
| 710 | 933.34 | 675.53 |
| 712 | 1576.36 | 2512.12 |
| 716 | 418.32 | 213.77 |
| 719 | 179.81 | 457.92 |
| 726 | 379.78 | 591.38 |
| 728 | 206.19 | 109.04 |
| 729 | 207.95 | 386.91 |
| 733 | 466.68 | 570.92 |
| 738 | 227.37 | 327.64 |
| 740 | 1593.31 | 967.69 |
| 741 | 1317.94 | 1525.5 |
| 742 | 396.52 | 251.43 |
| 745 | 366.95 | 780.22 |
| 749 | 170.59 | 486.26 |
| 752 | 852.35 | 1410.71 |
| 753 | 69.09 | 103.94 |
| 759 | 198.1 | 443.61 |
| 761 | 430.84 | 953.08 |
| 765 | 2889.47 | 4308.89 |
| 766 | 883.73 | 540.73 |
| 768 | 232.2 | 788.11 |
| 769 | 728.06 | 947.92 |
| 770 | 166.59 | 258.56 |
| 778 | 783.21 | 356.61 |
| 780 | 2863.23 | 733.37 |
| 781 | 370.95 | 153.69 |
| 798 | 465.94 | 390.57 |
| 802 | 1483.13 | 290.82 |
| 803 | 1420.86 | 378.98 |
| 809 | 490.19 | 295.85 |
| 810 | 1868.52 | 1489 |
| 811 | 421.44 | 311.34 |
| 819 | 1952.07 | 844.55 |
| 821 | 232.74 | 324.57 |
| 822 | 291.21 | 454.92 |
| 824 | 1711.53 | 1470.8 |
| 826 | 2857.72 | 2618.94 |
| 830 | 1733.28 | 9898.6 |
| 831 | 451.53 | 4337.06 |
| 836 | 2928.46 | 2173.05 |

Further, it is possible to recognize infiltrating BC by differential diagnosis. For the differential diagnosis between patients with no BC and patients with infiltrating BC, the frequencies according to Table 7 and the amplitudes according to Table 8 are suitable.

TABLE 7
Frequency markers for distinction between infiltrating BC
and patients with no BC

| | Occurrence in | Occurrence in |
|---|---|---|
| Number | group BC invasive | control |
| 1 | 0.44 | 0.04 |
| 3 | 0.89 | 0.34 |
| 4 | 0.76 | 0.14 |
| 7 | 0.95 | 0.37 |
| 11 | 0.58 | 0.17 |
| 16 | 0.44 | 0.01 |
| 23 | 0.84 | 0.14 |
| 27 | 0.89 | 0.3 |
| 28 | 0.56 | 0.02 |
| 29 | 0.53 | 0.07 |
| 30 | 0.76 | 0.16 |
| 31 | 0.93 | 0.46 |
| 34 | 0.6 | 0.13 |
| 39 | 0.76 | 0.24 |
| 91 | 0.4 | 0.88 |
| 112 | 0.75 | 0.34 |
| 132 | 0.25 | 0.86 |
| 134 | 0.27 | 0.8 |
| 179 | 0.71 | 0.24 |
| 180 | 0.64 | 0.18 |
| 186 | 0.62 | 0.16 |
| 187 | 0.73 | 0.19 |
| 189 | 0.53 | 0.06 |
| 192 | 0.42 | 0.01 |
| 194 | 0.44 | 0.02 |
| 200 | 0.62 | 0.14 |
| 204 | 0.71 | 0.2 |
| 205 | 0.76 | 0.3 |
| 206 | 0.73 | 0.3 |
| 207 | 0.6 | 0.11 |
| 209 | 0.53 | 0.04 |
| 211 | 0.84 | 0.28 |
| 213 | 0.51 | 0.02 |
| 215 | 0.58 | 0.07 |
| 216 | 0.71 | 0.21 |
| 228 | 0.49 | 0.06 |
| 229 | 0.55 | 0.07 |
| 230 | 0.51 | 0.08 |
| 231 | 0.45 | 0.02 |
| 232 | 0.78 | 0.24 |
| 233 | 0.73 | 0.23 |
| 234 | 0.45 | 0.05 |
| 235 | 0.69 | 0.25 |
| 240 | 0.71 | 0.1 |
| 248 | 0.49 | 0.09 |
| 255 | 0.85 | 0.38 |
| 259 | 0.6 | 0.19 |
| 260 | 0.71 | 0.21 |
| 264 | 0.73 | 0.24 |
| 267 | 0.53 | 0.02 |
| 270 | 0.56 | 0.11 |
| 277 | 0.84 | 0.39 |
| 279 | 0.71 | 0.18 |
| 280 | 0.69 | 0.17 |
| 282 | 0.8 | 0.12 |
| 287 | 0.4 | 0 |
| 316 | 0.58 | 0.13 |
| 325 | 0.47 | 0.04 |
| 327 | 0.62 | 0.2 |
| 330 | 0.51 | 0.02 |
| 338 | 0.44 | 0.02 |
| 345 | 0.55 | 0.06 |
| 361 | 0.47 | 0.02 |
| 365 | 0.58 | 0.07 |
| 368 | 0.51 | 0.01 |
| 372 | 0.53 | 0.02 |
| 377 | 0.62 | 0.13 |
| 383 | 0.71 | 0.12 |
| 390 | 0.69 | 0.2 |
| 394 | 0.2 | 0.61 |
| 400 | 0.27 | 0.75 |
| 407 | 0.55 | 0.12 |
| 413 | 0.33 | 0.88 |
| 415 | 0.6 | 0.19 |
| 425 | 0.24 | 0.69 |
| 426 | 0.04 | 0.47 |
| 433 | 0.67 | 0.19 |
| 436 | 0.07 | 0.48 |
| 440 | 0.2 | 0.68 |
| 442 | 0.53 | 0.02 |
| 451 | 0.13 | 0.62 |
| 466 | 0.71 | 0.27 |
| 469 | 0.56 | 0.04 |
| 474 | 0.2 | 0.66 |
| 477 | 0.24 | 0.71 |
| 485 | 0.58 | 0.15 |
| 494 | 0.56 | 0.07 |
| 496 | 0.24 | 0.67 |
| 497 | 0.67 | 0.16 |
| 508 | 0.56 | 0.08 |
| 525 | 0.47 | 0.06 |
| 526 | 0.49 | 0.03 |
| 533 | 0.16 | 0.59 |
| 539 | 0.05 | 0.46 |
| 542 | 0.45 | 0.06 |
| 557 | 0.58 | 0.15 |
| 560 | 0.51 | 0.06 |
| 561 | 0.53 | 0.07 |
| 567 | 0.29 | 0.73 |
| 576 | 0.15 | 0.57 |
| 580 | 0.78 | 0.24 |
| 581 | 0.55 | 0.14 |
| 582 | 0.64 | 0.2 |
| 583 | 0.47 | 0.92 |
| 591 | 0.25 | 0.7 |
| 594 | 0.67 | 0.22 |
| 596 | 0.55 | 0.07 |
| 613 | 0.45 | 0.06 |
| 620 | 0.64 | 0.07 |
| 629 | 0.49 | 0.01 |
| 635 | 0.73 | 0.22 |
| 644 | 0.44 | 0.04 |
| 655 | 0.27 | 0.73 |
| 685 | 0.87 | 0.34 |
| 687 | 0.55 | 0.07 |
| 691 | 0.67 | 0.16 |
| 692 | 0.75 | 0.2 |
| 695 | 0.4 | 0.81 |
| 708 | 0.75 | 0.07 |
| 709 | 0.69 | 0.28 |
| 712 | 0.33 | 0.76 |
| 713 | 0.58 | 0.02 |
| 718 | 0.47 | 0.02 |
| 721 | 0.42 | 0.02 |
| 724 | 0.49 | 0.07 |
| 736 | 0.53 | 0.06 |
| 746 | 0.78 | 0.22 |
| 750 | 0.6 | 0.14 |
| 757 | 0.56 | 0.16 |
| 759 | 0.16 | 0.6 |
| 762 | 0.6 | 0.2 |
| 771 | 0.73 | 0.22 |
| 779 | 0.47 | 0.04 |
| 780 | 0.78 | 0.33 |
| 788 | 0.53 | 0.04 |
| 791 | 0.49 | 0.04 |
| 802 | 0.67 | 0.27 |
| 803 | 0.71 | 0.28 |
| 804 | 0.71 | 0.14 |
| 806 | 0.51 | 0.07 |
| 807 | 0.53 | 0.06 |
| 808 | 0.73 | 0.18 |
| 825 | 0.55 | 0.07 |

TABLE 8
Amplitude markers for distinction between infiltrating
BC and patients with no BC

| | Mean | |
|---|---|---|
| | amplitude in | Mean |
| | group BC | amplitude in |
| Number | infiltrating | controls |
| 5 | 681.6 | 915.74 |
| 6 | 815.21 | 734.8 |
| 9 | 1243.87 | 782.89 |
| 10 | 141.18 | 124.95 |
| 21 | 1635.84 | 1958.08 |
| 32 | 397.54 | 254.21 |
| 37 | 446.42 | 159.1 |
| 40 | 1989.31 | 159.59 |
| 41 | 392 | 216.08 |
| 43 | 222.66 | 135.13 |
| 44 | 580.55 | 943.28 |
| 46 | 512.02 | 1158.64 |
| 48 | 425.9 | 664.52 |
| 52 | 4960.1 | 2173.05 |
| 53 | 433.44 | 537.27 |
| 56 | 1116.75 | 2641.97 |
| 57 | 366.09 | 577.01 |
| 63 | 189.75 | 318.87 |
| 66 | 1298.04 | 761.77 |
| 69 | 413.77 | 604.54 |
| 72 | 275.44 | 190.05 |
| 78 | 1447.52 | 957.12 |
| 80 | 117.88 | 139.63 |
| 81 | 716.39 | 1083.72 |
| 85 | 327.7 | 495.82 |
| 88 | 429.34 | 216.08 |
| 92 | 373.18 | 1464.36 |
| 93 | 380.46 | 315.1 |
| 101 | 239.84 | 206.42 |
| 111 | 99.21 | 241.12 |
| 113 | 695.63 | 1862.59 |
| 114 | 377.09 | 503.56 |
| 115 | 179.69 | 303.62 |
| 117 | 192.12 | 324.63 |
| 118 | 480.66 | 608.18 |
| 126 | 1277.47 | 764.74 |
| 130 | 126.13 | 352.84 |
| 140 | 188.41 | 276.45 |
| 145 | 353.26 | 183.44 |
| 148 | 118.89 | 351.18 |
| 149 | 1146.83 | 1740.05 |
| 151 | 5196.35 | 10498.07 |
| 152 | 433.72 | 272.96 |
| 153 | 9705.5 | 20785.64 |
| 154 | 1117.92 | 807.4 |
| 155 | 2216.16 | 4792.75 |
| 156 | 1782.53 | 2810.21 |
| 157 | 13518.15 | 5970.9 |
| 158 | 931.68 | 1996.73 |
| 159 | 4992.07 | 13360.51 |
| 160 | 3493.09 | 2426.25 |
| 161 | 4055.4 | 3023.97 |
| 162 | 8398.05 | 5176.62 |
| 163 | 2593.57 | 863.5 |
| 164 | 2038.97 | 484.44 |
| 165 | 357.58 | 558.57 |
| 166 | 1145.96 | 2233.02 |
| 169 | 352.85 | 86.39 |
| 170 | 105.05 | 224.3 |
| 173 | 355.05 | 717.77 |
| 174 | 336.82 | 673.1 |
| 175 | 1222.79 | 2369.24 |
| 176 | 845.59 | 869.56 |
| 178 | 324.58 | 744.72 |
| 179 | 439.74 | 900.41 |
| 180 | 1096.91 | 2012.33 |
| 182 | 832.52 | 1599.5 |
| 183 | 968.19 | 1591.47 |
| 188 | 47.58 | 19.49 |
| 190 | 118.25 | 235.42 |
| 191 | 79.98 | 33.56 |
| 193 | 192.29 | 123.78 |
| 195 | 95.67 | 45.2 |
| 196 | 90.6 | 720.98 |
| 197 | 127.3 | 85.68 |
| 199 | 583.69 | 319.41 |
| 201 | 107.79 | 57.89 |
| 202 | 277.13 | 119.03 |
| 203 | 135.81 | 90.37 |
| 208 | 374.07 | 70.68 |
| 210 | 815.93 | 617.85 |
| 212 | 197.69 | 287.56 |
| 214 | 286.24 | 127.03 |
| 217 | 495.39 | 292.45 |
| 218 | 149.46 | 93.76 |
| 220 | 542.41 | 309.18 |
| 221 | 175.55 | 120.89 |
| 222 | 178.86 | 145.69 |
| 223 | 134.7 | 31.02 |
| 224 | 90.56 | 308.11 |
| 226 | 2547.02 | 1141.14 |
| 227 | 1599.83 | 1031.48 |
| 236 | 619.61 | 237.07 |
| 238 | 409.42 | 56.43 |
| 241 | 226.47 | 151.59 |
| 242 | 5691.93 | 4175.12 |
| 243 | 436.57 | 189.83 |
| 244 | 415.21 | 170.6 |
| 245 | 260.16 | 141.51 |
| 246 | 200.04 | 124.78 |
| 247 | 96.98 | 88.51 |
| 250 | 477.46 | 72.76 |
| 251 | 330.58 | 245.38 |
| 252 | 141.41 | 134.05 |
| 253 | 608.95 | 239.5 |
| 254 | 242.32 | 315.01 |
| 256 | 510.44 | 342.62 |
| 257 | 283 | 176.66 |
| 258 | 103.68 | 45.1 |
| 262 | 1237.68 | 1993.54 |
| 263 | 437.43 | 284.59 |
| 265 | 682 | 451.29 |
| 268 | 374.36 | 200.42 |
| 269 | 675.02 | 421.74 |
| 273 | 1603.17 | 1707.78 |
| 274 | 253.2 | 166.84 |
| 276 | 371.26 | 44.14 |
| 278 | 1001.65 | 684.34 |
| 281 | 243.62 | 145.14 |
| 283 | 160.68 | 91.52 |
| 284 | 337.03 | 474.54 |
| 285 | 2505.38 | 72.28 |
| 286 | 2549.36 | 150.4 |
| 289 | 467.03 | 1024.9 |
| 290 | 186.3 | 79.4 |
| 291 | 558.3 | 287.08 |
| 292 | 9841.21 | 6153.2 |
| 293 | 414.98 | 327.89 |
| 294 | 348.42 | 151 |
| 295 | 1234.05 | 860.46 |
| 296 | 783.79 | 564.48 |
| 297 | 213.75 | 298.42 |
| 298 | 840.15 | 58.53 |
| 300 | 1990.34 | 1215.02 |
| 302 | 671.08 | 1913.27 |
| 304 | 194.42 | 55.41 |
| 305 | 172.63 | 73.15 |
| 308 | 147.74 | 102.43 |
| 309 | 867.46 | 497.12 |
| 310 | 1133.99 | 2939.46 |
| 313 | 1096.17 | 201.72 |
| 314 | 1191.03 | 699.24 |
| 315 | 162.8 | 115.01 |
| 317 | 1710.2 | 932.91 |
| 319 | 818.29 | 204.66 |
| 320 | 874.9 | 396.08 |
| 321 | 699.24 | 342.54 |
| 322 | 135.37 | 279.53 |
| 323 | 487.82 | 252.99 |
| 324 | 2230.59 | 166.99 |
| 326 | 857.23 | 362.46 |
| 328 | 377.72 | 156.8 |
| 329 | 387.05 | 238 |
| 331 | 343.69 | 198.75 |
| 333 | 8726.36 | 12695.31 |
| 335 | 798.51 | 415.43 |
| 336 | 205.47 | 121.63 |
| 337 | 221.04 | 258.72 |
| 339 | 97.95 | 181.81 |
| 342 | 432.51 | 329.69 |
| 343 | 1621.95 | 2575.03 |
| 344 | 614.12 | 309.71 |
| 346 | 441.7 | 631.08 |
| 348 | 194.86 | 126.88 |
| 349 | 1762.38 | 2743.26 |
| 350 | 7179.32 | 341.71 |
| 351 | 18950.35 | 33275.83 |
| 352 | 3233.23 | 4832.9 |
| 353 | 280.53 | 178.61 |
| 354 | 205.51 | 600.85 |
| 355 | 489.54 | 660.69 |
| 356 | 212.11 | 381.49 |
| 357 | 945.98 | 610.28 |
| 358 | 294.04 | 233.81 |
| 359 | 3084.9 | 1071.44 |
| 360 | 2474.74 | 3145.77 |
| 362 | 138.38 | 296.81 |
| 363 | 632.9 | 232.16 |
| 364 | 639.09 | 691.79 |
| 369 | 3411.18 | 5258.01 |
| 370 | 11667.49 | 21376.25 |
| 371 | 725.25 | 250.02 |
| 376 | 283.9 | 878.46 |
| 378 | 728.83 | 552.92 |
| 379 | 411.32 | 628.41 |
| 380 | 276.41 | 480.68 |
| 381 | 13594.59 | 253.26 |
| 382 | 1797.04 | 2562.08 |
| 384 | 74.02 | 94.63 |
| 386 | 459.1 | 356.62 |
| 388 | 649.43 | 2616.54 |
| 391 | 1297.86 | 2790.5 |
| 392 | 538.4 | 330.15 |
| 396 | 1118.64 | 1548.95 |
| 397 | 255.21 | 483.71 |
| 398 | 122.48 | 140.29 |
| 399 | 1368.03 | 696.02 |
| 401 | 368.33 | 999.31 |
| 402 | 4724.92 | 7951.13 |
| 403 | 1526.84 | 2730.17 |
| 405 | 702.99 | 1580.08 |
| 406 | 167.28 | 343.53 |
| 408 | 142.98 | 264.47 |
| 410 | 250.12 | 477.51 |
| 411 | 241.86 | 416.12 |
| 412 | 1103.38 | 1619.64 |
| 414 | 141.03 | 300.63 |
| 416 | 999.69 | 1460.35 |
| 417 | 800.56 | 1192.4 |
| 418 | 1088.84 | 1967.46 |
| 419 | 1559.36 | 1006.41 |
| 420 | 153.22 | 251.93 |
| 421 | 686.69 | 467.84 |
| 422 | 400.49 | 817.36 |
| 423 | 878.93 | 1580.15 |
| 424 | 443.24 | 641.55 |
| 427 | 230.97 | 122.78 |
| 428 | 1576.37 | 2794.73 |
| 429 | 2145.42 | 3482.39 |
| 431 | 252.94 | 190.7 |
| 432 | 151.34 | 269.09 |
| 434 | 773.88 | 1103.05 |
| 437 | 788.23 | 1328.66 |
| 438 | 1217.92 | 2565.32 |
| 439 | 200.2 | 346.97 |
| 441 | 1957.36 | 2749.89 |
| 443 | 465.87 | 412.54 |
| 444 | 272.39 | 110.58 |
| 445 | 245.78 | 464.12 |
| 447 | 281.31 | 244.14 |
| 449 | 76.2 | 220.69 |
| 450 | 750.55 | 378.52 |
| 453 | 139.55 | 344.87 |
| 454 | 369.2 | 851.23 |
| 455 | 85.25 | 132.39 |
| 456 | 106.58 | 159.23 |
| 457 | 212.57 | 278.24 |
| 458 | 1516.3 | 2420.4 |
| 459 | 1588.11 | 2722.3 |
| 461 | 287.43 | 487.39 |
| 462 | 1790.75 | 1405.3 |
| 463 | 432.2 | 310.42 |
| 465 | 2273.09 | 2831.8 |
| 467 | 300.32 | 170.89 |
| 468 | 277.28 | 315.21 |
| 470 | 1748.3 | 366.74 |
| 472 | 234.2 | 528.19 |
| 473 | 1735.46 | 2593 |
| 475 | 345.34 | 113.11 |
| 476 | 250.43 | 117.03 |
| 478 | 719.07 | 390.57 |
| 479 | 849.79 | 507.64 |
| 480 | 568.31 | 240.49 |
| 484 | 488.92 | 192.21 |
| 486 | 714.64 | 479.07 |
| 487 | 779.62 | 407.72 |
| 489 | 862.45 | 149.83 |
| 490 | 69.53 | 147.08 |
| 491 | 254.43 | 282.04 |
| 492 | 402.39 | 507.47 |
| 493 | 941.73 | 210.4 |
| 495 | 257.49 | 450.84 |
| 498 | 257.46 | 431.46 |
| 500 | 502.69 | 61.58 |
| 501 | 1854.99 | 707.59 |
| 502 | 137.49 | 169.93 |
| 503 | 140.4 | 252.44 |
| 504 | 203.04 | 131.9 |
| 505 | 84.63 | 139.9 |
| 506 | 184.34 | 123.01 |
| 507 | 2296.38 | 1347.3 |
| 509 | 477.71 | 241.64 |
| 510 | 425.28 | 197.63 |
| 511 | 995.99 | 1860.1 |
| 512 | 1078.39 | 213.78 |
| 513 | 66.83 | 235.62 |
| 514 | 358.26 | 78.89 |
| 515 | 4934.49 | 943.15 |
| 516 | 510.22 | 154.31 |
| 517 | 790.5 | 344.83 |
| 518 | 862.14 | 120.51 |
| 520 | 976.83 | 589.3 |
| 521 | 1091.43 | 410.04 |
| 524 | 124.24 | 287.18 |
| 527 | 127.3 | 313.99 |
| 528 | 122.54 | 162.9 |
| 529 | 543.24 | 654.44 |
| 530 | 87.72 | 136.71 |
| 532 | 1066.35 | 725.68 |
| 534 | 1202.07 | 1722.5 |
| 537 | 258.42 | 252.33 |
| 538 | 305.95 | 133.39 |
| 541 | 294.45 | 368.78 |
| 543 | 139.85 | 522.74 |
| 546 | 410.38 | 179.19 |
| 548 | 296.88 | 126.04 |
| 549 | 550.37 | 207.91 |
| 550 | 228.52 | 764.16 |
| 551 | 512.6 | 290.72 |
| 552 | 297.73 | 252.04 |
| 553 | 107.65 | 63.73 |
| 554 | 925.07 | 559.35 |
| 555 | 226.74 | 131.69 |
| 556 | 3423.44 | 211.72 |
| 558 | 281.61 | 274.93 |
| 559 | 145.28 | 351.22 |
| 562 | 648.04 | 1019.2 |
| 563 | 670.49 | 202.92 |
| 565 | 758.68 | 484.55 |
| 568 | 779.64 | 127.81 |
| 569 | 836.94 | 671.47 |
| 570 | 258.18 | 74.14 |
| 571 | 944.81 | 306.21 |
| 572 | 526.09 | 215.39 |
| 573 | 265.12 | 561.18 |
| 574 | 318.19 | 165.07 |
| 575 | 1531.87 | 1267 |
| 577 | 18967.25 | 840.95 |
| 578 | 210.08 | 510.51 |
| 579 | 509.75 | 219.86 |
| 584 | 181.19 | 618.79 |
| 585 | 759.09 | 217.1 |
| 586 | 4565.75 | 58.99 |
| 587 | 191.97 | 218.19 |
| 588 | 192.37 | 103.5 |
| 589 | 263 | 405.5 |
| 590 | 3057.26 | 126.77 |
| 592 | 184.87 | 539.84 |
| 593 | 135.07 | 411.88 |
| 595 | 674.57 | 132.06 |
| 597 | 309.64 | 679.53 |
| 598 | 1239.18 | 168.71 |
| 603 | 3414.12 | 1369.9 |
| 604 | 962.06 | 169.59 |
| 606 | 3896.53 | 8436 |
| 607 | 111.01 | 126.02 |
| 608 | 7801.67 | 886.26 |
| 610 | 200.22 | 2017.3 |
| 611 | 1670.86 | 90.16 |
| 612 | 207.94 | 158.12 |
| 614 | 152.15 | 342.56 |
| 616 | 627.87 | 979.18 |
| 617 | 549.7 | 1001.2 |
| 619 | 183.49 | 457.6 |
| 621 | 191.66 | 223.31 |
| 622 | 3449.9 | 6523.9 |
| 623 | 182 | 256.33 |
| 624 | 577.49 | 913.29 |
| 625 | 62.18 | 168.82 |
| 626 | 244.08 | 497.04 |
| 627 | 2052.37 | 858.44 |
| 628 | 2563.18 | 274.04 |
| 630 | 103.25 | 158.48 |
| 632 | 317.32 | 93 |
| 633 | 3020.85 | 1120.7 |
| 634 | 329.03 | 786.49 |
| 637 | 364.89 | 197.87 |
| 639 | 275.25 | 140.43 |
| 640 | 115.65 | 66.25 |
| 641 | 498.77 | 420.21 |
| 642 | 414.68 | 199.62 |
| 645 | 779.48 | 334.7 |
| 647 | 5050.96 | 3358.3 |
| 648 | 175.97 | 294.91 |
| 649 | 699.27 | 200.78 |
| 651 | 2586.25 | 1291.1 |
| 652 | 231.51 | 143.66 |
| 653 | 915.81 | 1935 |
| 654 | 5467.07 | 3725.6 |
| 656 | 819.99 | 386.71 |
| 659 | 141.99 | 980.59 |
| 660 | 3289.37 | 329.08 |
| 661 | 1919.62 | 2717 |
| 662 | 250.8 | 623.37 |
| 663 | 81.8 | 189.73 |
| 664 | 122.34 | 1413.9 |
| 665 | 737.4 | 407.33 |
| 666 | 426.8 | 770.9 |
| 667 | 299.35 | 416.59 |
| 668 | 188.12 | 612.28 |
| 669 | 387.84 | 768.38 |
| 671 | 587.68 | 897.03 |
| 672 | 368.29 | 117.39 |
| 673 | 21397.52 | 34.43 |
| 674 | 187.19 | 256.29 |
| 675 | 665.34 | 927.95 |
| 677 | 892.27 | 307.55 |
| 678 | 989.87 | 1826.7 |
| 679 | 1128.17 | 1729 |
| 680 | 851.56 | 1493.4 |
| 681 | 1136.29 | 621.15 |
| 683 | 1148.54 | 504.81 |
| 686 | 1363.26 | 797.98 |
| 688 | 98.69 | 156.14 |
| 689 | 53.97 | 120.23 |
| 690 | 132.93 | 196.27 |
| 693 | 843.36 | 485.58 |
| 694 | 2694.55 | 1306 |
| 696 | 586.14 | 227.88 |
| 698 | 960.67 | 1170 |
| 699 | 218.88 | 140.39 |
| 700 | 68.12 | 132.51 |
| 701 | 1882.74 | 2710.4 |
| 702 | 709.66 | 988.21 |
| 703 | 465.93 | 247.99 |
| 704 | 452.19 | 552.79 |
| 707 | 157.49 | 209.94 |
| 708 | 157.54 | 1118.7 |
| 711 | 132.71 | 286.79 |
| 714 | 543.08 | 281.17 |
| 715 | 644.42 | 1159 |
| 717 | 612.86 | 987.76 |
| 720 | 116.14 | 91.86 |
| 722 | 447.59 | 2446.05 |
| 723 | 13940.67 | 18395.9 |
| 725 | 187.68 | 334.29 |
| 726 | 397.81 | 591.38 |
| 727 | 637.1 | 1016.73 |
| 728 | 65.58 | 109.04 |
| 730 | 671.33 | 1689.36 |
| 731 | 110.46 | 373.29 |
| 732 | 3464.04 | 934.95 |
| 733 | 556.67 | 570.92 |
| 734 | 215.66 | 410.25 |
| 735 | 622.74 | 321.95 |
| 737 | 130 | 213.43 |
| 738 | 190.04 | 327.64 |
| 739 | 293.28 | 366.38 |
| 743 | 185.63 | 332.04 |
| 744 | 819.18 | 507.03 |
| 745 | 602.42 | 780.22 |
| 747 | 347.49 | 178.71 |
| 748 | 410.63 | 345.85 |
| 749 | 137.85 | 486.26 |
| 751 | 1303.86 | 1883.97 |
| 754 | 822.75 | 398.24 |
| 755 | 6713.94 | 2358.14 |
| 756 | 166.21 | 284.44 |
| 758 | 200.24 | 250.67 |
| 760 | 1216.67 | 2134.8 |
| 763 | 399.33 | 320.2 |
| 764 | 128.33 | 248.54 |
| 765 | 1514.46 | 4308.89 |
| 767 | 293.5 | 190.8 |
| 768 | 283.27 | 788.11 |
| 769 | 1403.56 | 947.92 |
| 770 | 418.46 | 258.56 |
| 772 | 1313.89 | 627.71 |
| 773 | 211.83 | 927.49 |
| 774 | 1337.79 | 550.41 |
| 775 | 244.98 | 490.03 |
| 776 | 3574.99 | 7395.44 |
| 777 | 164.47 | 292.43 |
| 778 | 387.48 | 356.61 |
| 781 | 1038.97 | 153.69 |
| 782 | 4315.89 | 6177.03 |
| 783 | 916.46 | 441.9 |
| 784 | 1412.71 | 1241.77 |
| 785 | 634.19 | 106.33 |
| 786 | 78.13 | 220.29 |
| 787 | 413.79 | 34.69 |
| 789 | 814.07 | 750.37 |
| 790 | 1584 | 4381.9 |
| 792 | 92.04 | 51.86 |
| 793 | 1306.38 | 777.08 |
| 794 | 125.55 | 184.79 |
| 795 | 825.81 | 1314.25 |
| 796 | 456.95 | 140.52 |
| 797 | 461.61 | 182.5 |
| 799 | 437.29 | 564.91 |
| 800 | 448.25 | 288.48 |
| 801 | 165.93 | 148.57 |
| 805 | 634.21 | 454.65 |
| 809 | 679.06 | 295.85 |
| 811 | 618.77 | 311.34 |
| 812 | 1119.36 | 388.74 |
| 813 | 145.77 | 66.69 |
| 814 | 864.31 | 859.71 |
| 815 | 638.46 | 331.57 |
| 816 | 1143.81 | 743.86 |
| 817 | 1557.62 | 753.52 |
| 818 | 1908.62 | 1216.08 |
| 819 | 4979.79 | 844.55 |
| 820 | 217.82 | 155.57 |
| 823 | 336.34 | 689.88 |
| 824 | 3252.92 | 1470.8 |
| 826 | 5471.02 | 2618.94 |
| 827 | 2845.32 | 1552.78 |
| 828 | 1179.08 | 575.57 |
| 829 | 1035.75 | 505.99 |
| 830 | 2085.83 | 9898.6 |
| 832 | 1003.08 | 2095.65 |
| 833 | 6617.56 | 1583.61 |
| 834 | 670.25 | 313.23 |
| 835 | 1014.66 | 600.48 |

The markers according to the disclosure can also be employed for the differential diagnosis of the individual stages pTa to pT4.

The subject from which the sample in which the presence or absence or the amplitude of one or more polypeptide markers is determined is derived may be any subject which is capable of suffering from bladder cancer, for example, an animal or human. Preferably, the subject is a mammal, and most preferably, it is a human.

In a preferred embodiment of the disclosure, not just one polypeptide marker, but a combination of markers are used to diagnose bladder cancer, wherein the existence of bladder cancer is concluded from their presence or absence and/or the height of the amplitude. By comparing a plurality of polypeptide markers, a bias in the overall result from a few individual deviations from the typical presence probability in the sick or control individual can be reduced or avoided.

The sample in which the presence or absence or amplitude of the polypeptide marker or markers according to the disclosure is measured may be any sample which is obtained from the body of the subject. The sample is a sample which has a polypeptide composition suitable for providing information about the state of the subject (bladder cancer or not). For example, it may be blood, urine, synovial fluid, tissue fluid, a body secretion, sweat, cerebrospinal fluid, lymph, intestinal, gastric, pancreatic juices, bile, lachrymal fluid, a tissue sample, sperm, vaginal fluid or a faeces specimen. Preferably, it is a liquid sample.

In a preferred embodiment, the sample is a urine sample or blood sample,

In a preferred embodiment, the sample is a urine sample or blood sample, wherein said blood sample may be a (blood) serum or (blood) plasma sample.

Urine samples can be taken as known in the prior art. Preferably, a midstream urine sample is used in the context of the present disclosure. For example, the urine sample may be taken by means of a catheter or also by means of an urination apparatus as described in WO 01/74275.

Blood samples can be taken by methods known in the prior art, for example, from a vein, artery or capillary. Usually, a blood sample is obtained by withdrawing venous blood by means of a syringe, for example, from an arm of the subject. The term “blood sample” includes samples obtained from blood by further purification and separation methods, such as blood plasma or blood serum.

The presence or absence of a polypeptide marker in the sample may be determined by any method known in the prior art that is suitable for measuring polypeptide markers. Such methods are known to the skilled person. In principle, the presence or absence of a polypeptide marker can be determined by direct methods, such as mass spectrometry, or indirect methods, for example, by means of ligands.

If required or desirable, the sample from the subject, for example, the urine or blood sample, may be pretreated by any suitable means and, for example, purified or separated before the presence or absence of the polypeptide marker or markers is measured. The treatment may comprise, for example, purification, separation, dilution or concentration. The methods may be, for example, centrifugation, filtration, ultrafiltration, dialysis, precipitation or chromatographic methods, such as affinity separation or separation by means of ion-exchange chromatography, or electrophoretic separation. Particular examples thereof are gel electrophoresis, two-dimensional polyacrylamide gel electrophoresis (2D-PAGE), capillary electrophoresis, metal affinity chromatography, immobilized metal affinity chromatography (IMAC), lectin-based affinity chromatography, liquid chromatography, high-performance liquid chromatography (HPLC), normal and reverse-phase HPLC, cation-exchange chromatography and selective binding to surfaces. All these methods are well known to the skilled person, and the skilled person will be able to select the method as a function of the sample employed and the method for determining the presence or absence of the polypeptide marker or markers.

In one embodiment of the disclosure, the sample, before being measured, is separated by capillary electrophoresis, purified by ultracentrifugation and/or divided by ultrafiltration into fractions which contain polypeptide markers of a particular molecular size.

Preferably, a mass-spectrometric method is used to determine the presence or absence of a polypeptide marker, wherein a purification or separation of the sample may be performed upstream from such method. As compared to the currently employed methods, mass-spectrometric analysis has the advantage that the concentration of many (>100) polypeptides of a sample can be determined by a single analysis. Any type of mass spectrometer may be employed. By means of mass spectrometry, it is possible to measure 10 fmol of a polypeptide marker, i.e., 0.1 ng of a 10 kDa protein, as a matter of routine with a measuring accuracy of about ±0.01% in a complex mixture. In mass spectrometers, an ion-forming unit is coupled with a suitable analytic device. For example, electrospray-ionization (ESI) interfaces are mostly used to measure ions in liquid samples, whereas the matrix-assisted laser desorption/ionization (MALDI) technique is used for measuring ions from a sample crystallized with a matrix. For analyzing the ions formed, quadrupoles, ion traps or time-of-flight (TOF) analyzers may be used.

In electrospray ionization (ESI), the molecules present in solution are atomized, inter alia, under the influence of high voltage (e.g., 1-8 kV), which forms charged droplets that become smaller from the evaporation of the solvent. Finally, so-called Coulomb explosions cause the formation of free ions, which can then be analyzed and detected.

In the analysis of the ions by means of TOF, a particular acceleration voltage is applied which confers an equal amount of kinetic energy to the ions. Thereafter, the time that the respective ions take to travel a particular drifting distance through the flying tube is measured very accurately. Since with equal amounts of kinetic energy, the velocity of the ions depends on their mass, the latter can thus be determined. TOF analyzers have a very high scanning speed and therefore reach a very high resolution.

Preferred methods for the determination of the presence and absence of polypeptide markers include gas-phase ion spectrometry, such as laser desorption/ionization mass spectrometry, MALDI-TOF MS, SELDI-TOF MS (surface-enhanced laser desorption/ionization), LC-MS (liquid chromatography/mass spectrometry), 2D-PAGE/MS and capillary electrophoresis-mass spectrometry (CE-MS). All methods mentioned are known to the skilled person.

A particularly preferred method is CE-MS, in which capillary electrophoresis is coupled with mass spectrometry. This method has been described in some detail, for example, in the German Patent Application DE 10021737, in Kaiser et al. (J Chromatogr A, 2003, Vol. 1013: 157-171, and Electrophoresis, 2004, 25: 2044-2055) and in Wittke et al. (Journal of Chromatography A, 2003, 1013: 173-181). The CE-MS technology allows to determine the presence of some hundreds of polypeptide markers of a sample simultaneously within a short time and in a small volume with high sensitivity. After a sample has been measured, a pattern of the measured polypeptide markers is prepared. This pattern can be compared with reference patterns of sick or healthy subjects. In most cases, it is sufficient to use a limited number of polypeptide markers for the diagnosis of bladder cancer and the differential diagnosis between different stages of bladder cancer, for example, at least 6, 8, 10, 20, 50 or 100 markers.

A CE-MS method which includes CE coupled on-line to an ESI-TOF MS device is further preferred.

For CE-MS, the use of volatile solvents is preferred, and it is best to work under essentially salt-free conditions. Examples of suitable solvents include acetonitrile, methanol and the like. The solvents can be diluted with water or admixed with a weak acid (e.g., from 0.10% to 1% formic acid) in order to protonate the analyte, preferably the polypeptides.

By means of capillary electrophoresis, it is possible to separate molecules by their charge and size. Neutral particles will migrate at the speed of the electroosmotic flow upon application of a current, while cations are accelerated towards the cathode, and anions are delayed. The advantage of capillaries in electrophoresis resides in their favorable ratio of surface to volume, which enables a good dissipation of the Joule heat generated during the current flow. This in turn allows high voltages (usually up to 30 kV) to be applied and thus a high separating performance and short times of analysis.

In capillary electrophoresis, silica glass capillaries having inner diameters of typically from 50 to 75 μm are usually employed. The lengths employed are from 30 to 100 cm. In addition, the capillaries are usually made of plastic-coated silica glass. The capillaries may be both untreated, i.e., expose their hydrophilic groups on the interior surface, or coated on the interior surface. A hydrophobic coating (coating: a method that conceals the negative polarized surface of silica, for example) may be used to improve the resolution. In addition to the voltage, a pressure may also be applied, which typically is within a range of from 0 to 1 psi. The pressure may also be applied only during the performance or altered meanwhile.

In a preferred method for measuring polypeptide markers, the markers of the sample are separated by means of capillary electrophoresis, then directly ionized and transferred on-line to a mass spectrometer coupled thereto for detection.

In the method according to the disclosure, it is advantageous to use several polypeptide markers for the diagnosis of bladder cancer. In particular, at least three polypeptide markers may be used, for example, markers 1, 2 and 3; 1, 2 and 4; etc.

More preferred is the use of at least 4, 5 or 6 markers.

Even more preferred is the use of at least 15 markers, for example, markers 1 to 15.

Most preferred is the use of all markers listed in Tables 1 or 4.

In order to determine the probability of the existence of bladder cancer when several markers are used, statistic methods known to the skilled person may be used. For example, the Random Forests method described by Weissinger et al. (Kidney Int., 2004, 65: 2426-2434) by using a computer program such as S-Plus or the support vector machines described in the same publication may be used.

### Example

### 1. Sample Preparation

For detecting the polypeptide markers for bladder cancer, urine was employed. Urine was withdrawn from healthy donors (control group) as well as from patients suffering from bladder cancer.

For the subsequent CE-MS measurement, the proteins which are also contained in the urine of patients in a higher concentration, such as albumin and immunoglobulins, had to be separated off by ultrafiltration. Thus, 700 μl of urine was removed and admixed with 700 μl of filtration buffer (2 M urea, 10 mM ammonia, 0.02% SDS). This 1.4 ml of sample volume was ultrafiltrated (20 kDa, Sartorius, Göttingen, Germany). The ultrafiltration was performed at 3000 rpm in a centrifuge until 1.1 ml of ultrafiltrate was obtained.

The 1.1 ml of filtrate obtained was then applied to a PD 10 column (Amersham Bioscience, Uppsala, Sweden) and eluted with 2.5 ml of 0.010% NH₄OH, and lyophilized. For the CE-MS measurement, the polypeptides were then resuspended with 20 μl of water (HPLC grade, Merck).

### 2. CE-MS Measurement

The CE-MS measurements were performed with a capillary electrophoresis system from Beckman Coulter (P/ACE MDQ System; Beckman Coulter Inc., Fullerton, USA) and an ESI-TOF mass spectrometer from Bruker (micro-TOF MS, Bruker Daltonik, Bremen, Germany).

The CE capillaries were supplied by Beckman Coulter and had an ID/OD of 50/360 μm and a length of 90 cm. The mobile phase for the CE separation consisted of 20% acetonitrile and 0.25% formic acid in water. For the “sheath flow” on the MS, 300% isopropanol with 0.50% formic acid was used at a flow rate of 2 μl/min. The coupling of CE and MS was realized by a CE-ESI-MS Sprayer Kit (Agilent Technologies, Waldbronn, Germany).

For injecting the sample, a pressure of from 1 to a maximum of 6 psi was applied, and the duration of the injection was 99 seconds. With these parameters, about 150 to 900 nl of the sample was injected into the capillary, which corresponds to about 100% to 500% of the capillary volume. A stacking technique was used to concentrate the sample in the capillary. Thus, before the sample was injected, a 1 M NH₃ solution was injected for 7 seconds (at 1 psi), and after the sample was injected, a 2 M formic acid solution was injected for 5 seconds. After the separation voltage (30 kV) was applied, the analytes were automatically concentrated between these solutions.

The subsequent CE separation was performed with a pressure method: 40 minutes at 0 psi, then 0.1 psi for 2 min, 0.2 psi for 2 min, 0.3 psi for 2 min, 0.4 psi for 2 min, and finally 0.5 psi for 32 min. The total duration of a separation run was thus 80 minutes.

In order to obtain as good as possible a signal intensity on the side of the MS, the nebulizer gas was set to the lowest possible value. The voltage applied to the spray needle for generating the electrospray was 3700-4100 V. The remaining settings at the mass spectrometer were optimized for peptide detection according to the manufacturer's protocol. The spectra were recorded over a mass range of m/z 400 to m/z 3000 and accumulated every 3 seconds.

### 3. Standards for the CE Measurement

For checking and calibrating the CE measurement, the following proteins or polypeptides which are characterized by the stated CE migration times under the selected conditions were employed:

| Protein/polypeptide | Migration time |
|---|---|
| Aprotinin (SIGMA, Taufkirchen, DE, Cat. # A1153) | 9.2 min |
| Ribonuclease (SIGMA, Taufkirchen, DE, Cat. # R4875) | 10.9 min |
| Lysozyme (SIGMA, Taufkirchen, DE, Cat. # L7651) | 8.9 min |
| “REV”, Sequence: REVQSKIGYGRQIIS | 15.6 min |
| “ELM”, Sequence: ELMTGELPYSHINNRDQIIFMVGR | 23.4 min |
| “KINCON”, Sequence: TGSLPYSHIGSRDQIIFMVGR | 20.0 min |
| “GIVLY” Sequence: GIVLYELMTGELPYSHIN | 36.8 min |

The proteins/polypeptides were employed at a concentration of 10 pmol/μl each in water. “REV”, “ELM, “KINCON” and “GIVLY” are synthetic peptides.

The molecular masses of the peptides and the m/z ratios of the individual charge states visible in MS are listed in the following Table:

| | H (mono) | | | | | | |
|---|---|---|---|---|---|---|---|
| | 1.0079 | | | 1.0079 | 1.0079 | 1.0079 | 1.0079 |
| | Aprotinin | 1.0079 | 1.0079 | REV | KINCON | ELM | GIVLY |
| | Mono | Ribonuclease | Lysozyme | Mono | Mono | Mono | Mono |
| m/z | Mass | Mono Mass | Mono Mass | Mass | Mass | Mass | Mass |
| 0 | 6513.09 | 13681.32 | 14303.88 | 1732.96 | 2333.19 | 2832.41 | 2048.03 |
| 1 | 6514.0979 | 13682.328 | 14304.888 | 1733.9679 | 2334.1979 | 2833.4179 | 2049.0379 |
| 2 | 3257.5529 | 6841.6679 | 7152.9479 | 867.4879 | 1167.6029 | 1417.2129 | 1025.0229 |
| 3 | 2172.0379 | 4561.4479 | 4768.9679 | 578.6612 | 778.7379 | 945.1446 | 683.6846 |
| 4 | 1629.2804 | 3421.3379 | 3576.9779 | 434.2479 | 584.3054 | 709.1104 | 513.0154 |
| 5 | 1303.6259 | 2737.2719 | 2861.7839 | 347.5999 | 467.6459 | 567.4899 | 410.6139 |
| 6 | 1086.5229 | 2281.2279 | 2384.9879 | 289.8346 | 389.8729 | 473.0762 | 342.3462 |
| 7 | 931.4494 | 1955.4822 | 2044.4193 | 248.5736 | 334.3208 | 405.6379 | 293.5836 |
| 8 | 815.1442 | 1711.1729 | 1788.9929 | 217.6279 | 292.6567 | 355.0592 | 257.0117 |
| 9 | 724.6846 | 1521.1546 | 1590.3279 | 193.559 | 260.2512 | 315.7201 | 228.5668 |
| 10 | 652.3169 | 1369.1399 | 1431.3959 | 174.3039 | 234.3269 | 284.2489 | 205.8109 |
| 11 | 593.107 | 1244.7643 | 1301.3606 | 158.5497 | 213.1161 | 258.4997 | 187.1924 |
| 12 | 543.7654 | 1141.1179 | 1192.9979 | 145.4212 | 195.4404 | 237.0421 | 171.6771 |
| 13 | 502.0148 | 1053.4171 | 1101.3063 | 134.3125 | 180.4841 | 218.8856 | 158.5486 |

1-18. (canceled)

19. A method for the diagnosis of bladder cancer (BC) and/or for determining a tumor stage of bladder cancer, comprising the step of determining the presence or absence or amplitude of at least six polypeptide markers in a sample, wherein said polypeptide markers are selected from markers 1 to 836, which are characterized by the following values for the molecular masses and migration times (CE time):

| Number | Mass | CE Time |
|---|---|---|
| 1 | 4933.97 | 17.2 |
| 2 | 4712.25 | 14.3 |
| 3 | 1071.54 | 16.3 |
| 4 | 3273.42 | 17.7 |
| 5 | 1130.37 | 33.3 |
| 6 | 1180.53 | 33.7 |
| 7 | 980.36 | 34.0 |
| 8 | 11721.36 | 13.4 |
| 9 | 2077.12 | 16.8 |
| 10 | 840.42 | 19.3 |
| 11 | 2115.04 | 22.8 |
| 12 | 3385.43 | 21.1 |
| 13 | 4053.18 | 13.6 |
| 14 | 4240.13 | 14.2 |
| 15 | 1387.04 | 9.5 |
| 16 | 2597.47 | 18.6 |
| 17 | 2920.21 | 15.9 |
| 18 | 4509.09 | 25.1 |
| 19 | 8763.45 | 12.1 |
| 20 | 1535.72 | 26.3 |
| 21 | 911.28 | 31.9 |
| 22 | 1191.54 | 34.5 |
| 23 | 3759.90 | 12.8 |
| 24 | 2973.43 | 20.0 |
| 25 | 3325.67 | 15.4 |
| 26 | 10042.81 | 12.2 |
| 27 | 1380.67 | 19.3 |
| 28 | 3502.70 | 12.5 |
| 29 | 3632.63 | 15.9 |
| 30 | 8559.22 | 13.2 |
| 31 | 4960.86 | 15.6 |
| 32 | 858.41 | 18.9 |
| 33 | 847.40 | 20.0 |
| 34 | 980.51 | 18.7 |
| 35 | 1128.52 | 22.4 |
| 36 | 1407.68 | 35.6 |
| 37 | 2093.92 | 31.0 |
| 38 | 2421.04 | 32.5 |
| 39 | 1250.66 | 14.8 |
| 40 | 3242.20 | 18.0 |
| 41 | 1040.50 | 21.2 |
| 42 | 3176.40 | 11.9 |
| 43 | 2778.55 | 32.5 |
| 44 | 1422.58 | 35.8 |
| 45 | 3524.42 | 28.9 |
| 46 | 3831.74 | 24.8 |
| 47 | 2488.79 | 32.2 |
| 48 | 1099.51 | 24.9 |
| 49 | 1936.82 | 29.2 |
| 50 | 1265.61 | 23.4 |
| 51 | 3081.61 | 26.4 |
| 52 | 3266.48 | 26.5 |
| 53 | 3821.95 | 20.4 |
| 54 | 2407.10 | 24.0 |
| 55 | 1596.75 | 19.0 |
| 56 | 10341.75 | 18.1 |
| 57 | 4306.66 | 28.9 |
| 58 | 900.43 | 19.8 |
| 59 | 1645.74 | 15.9 |
| 60 | 1844.58 | 31.4 |
| 61 | 2130.97 | 29.6 |
| 62 | 2359.35 | 31.0 |
| 63 | 3343.58 | 28.5 |
| 64 | 3501.62 | 28.4 |
| 65 | 5675.31 | 19.0 |
| 66 | 1312.64 | 17.6 |
| 67 | 1867.68 | 30.4 |
| 68 | 2186.85 | 31.9 |
| 69 | 2414.51 | 33.6 |
| 70 | 2739.24 | 24.8 |
| 71 | 2907.36 | 33.9 |
| 72 | 3031.42 | 34.0 |
| 73 | 4345.89 | 30.6 |
| 74 | 6211.67 | 14.6 |
| 75 | 1080.52 | 22.9 |
| 76 | 1142.80 | 10.9 |
| 77 | 1321.74 | 10.6 |
| 78 | 1750.77 | 19.5 |
| 79 | 1989.90 | 29.3 |
| 80 | 2946.38 | 32.8 |
| 81 | 4942.34 | 21.4 |
| 82 | 1668.76 | 17.5 |
| 83 | 1688.71 | 14.5 |
| 84 | 1731.79 | 17.7 |
| 85 | 1822.74 | 27.4 |
| 86 | 3870.83 | 30.4 |
| 87 | 4275.48 | 20.6 |
| 88 | 1025.48 | 21.7 |
| 89 | 1353.67 | 22.1 |
| 90 | 1466.67 | 25.2 |
| 91 | 1588.73 | 26.7 |
| 92 | 1878.72 | 27.6 |
| 93 | 2933.31 | 24.1 |
| 94 | 3338.45 | 19.3 |
| 95 | 6055.63 | 15.6 |
| 96 | 2266.00 | 17.2 |
| 97 | 2939.05 | 31.1 |
| 98 | 3021.39 | 18.9 |
| 99 | 1299.60 | 17.6 |
| 100 | 1405.68 | 14.3 |
| 101 | 1526.72 | 19.3 |
| 102 | 1630.78 | 15.4 |
| 103 | 1865.83 | 30.0 |
| 104 | 2687.24 | 25.4 |
| 105 | 2756.27 | 32.8 |
| 106 | 1200.84 | 10.7 |
| 107 | 1391.81 | 10.7 |
| 108 | 1487.68 | 26.1 |
| 109 | 1919.87 | 23.3 |
| 110 | 2063.48 | 14.1 |
| 111 | 2753.47 | 34.2 |
| 112 | 2864.16 | 28.5 |
| 113 | 3858.65 | 21.9 |
| 114 | 4015.96 | 24.4 |
| 115 | 1324.60 | 16.3 |
| 116 | 1793.60 | 15.4 |
| 117 | 2215.79 | 30.7 |
| 118 | 1934.82 | 14.2 |
| 119 | 1935.96 | 13.6 |
| 120 | 2062.81 | 22.9 |
| 121 | 13372.32 | 25.9 |
| 122 | 1495.68 | 18.9 |
| 123 | 1513.49 | 35.4 |
| 124 | 1900.90 | 28.6 |
| 125 | 3108.94 | 33.8 |
| 126 | 1860.87 | 16.2 |
| 127 | 2194.68 | 14.3 |
| 128 | 3956.81 | 21.3 |
| 129 | 1496.70 | 26.5 |
| 130 | 3659.17 | 21.3 |
| 131 | 13169.24 | 25.6 |
| 132 | 1608.75 | 27.5 |
| 133 | 1707.73 | 11.1 |
| 134 | 1592.72 | 17.3 |
| 135 | 2029.80 | 15.0 |
| 136 | 13007.18 | 25.1 |
| 137 | 2117.77 | 29.9 |
| 138 | 2168.87 | 31.0 |
| 139 | 1916.86 | 31.3 |
| 140 | 2802.78 | 34.7 |
| 141 | 1111.77 | 10.8 |
| 142 | 1806.84 | 18.5 |
| 143 | 1594.71 | 26.1 |
| 144 | 1778.76 | 27.0 |
| 145 | 2582.95 | 19.0 |
| 146 | 3605.63 | 21.22 |
| 147 | 3765.31 | 42.77 |
| 148 | 4067.63 | 31.22 |
| 149 | 10640.4 | 19.68 |
| 150 | 2577.35 | 20.9 |
| 151 | 3292.55 | 37.5 |
| 152 | 2058.90 | 18.8 |
| 153 | 3001.36 | 33.2 |
| 154 | 2157.04 | 17.0 |
| 155 | 2854.36 | 32.6 |
| 156 | 1653.90 | 26.9 |
| 157 | 2658.32 | 13.1 |
| 158 | 3657.65 | 38.7 |
| 159 | 1754.92 | 27.8 |
| 160 | 1467.82 | 19.8 |
| 161 | 1580.91 | 20.1 |
| 162 | 2563.89 | 17.6 |
| 163 | 2355.15 | 17.9 |
| 164 | 3891.40 | 20.2 |
| 165 | 1561.49 | 35.6 |
| 166 | 1576.63 | 23.1 |
| 167 | 1911.07 | 20.8 |
| 168 | 3265.47 | 19.8 |
| 169 | 2344.91 | 32.3 |
| 170 | 2743.98 | 32.8 |
| 171 | 3401.75 | 18.1 |
| 172 | 3425.87 | 26.1 |
| 173 | 5510.18 | 23.1 |
| 174 | 2356.70 | 33.5 |
| 175 | 3256.25 | 29.3 |
| 176 | 12716.23 | 23.1 |
| 177 | 1050.49 | 23.3 |
| 178 | 1950.87 | 33.9 |
| 179 | 1649.76 | 17.9 |
| 180 | 1312.55 | 26.3 |
| 181 | 1438.47 | 35.2 |
| 182 | 3248.51 | 26.8 |
| 183 | 742.26 | 25.3 |
| 184 | 981.60 | 20.7 |
| 185 | 1679.97 | 19.3 |
| 186 | 801.43 | 22.7 |
| 187 | 816.41 | 21.05 |
| 188 | 818.48 | 21.95 |
| 189 | 838.36 | 25.12 |
| 190 | 838.44 | 35.05 |
| 191 | 840.47 | 19.68 |
| 192 | 852.47 | 20.42 |
| 193 | 868.45 | 23.35 |
| 194 | 871.48 | 21.11 |
| 195 | 875.51 | 21.83 |
| 196 | 876.43 | 35.13 |
| 197 | 883.45 | 23.27 |
| 198 | 884.36 | 24.91 |
| 199 | 892.32 | 35.22 |
| 200 | 906.34 | 34.93 |
| 201 | 906.51 | 21.89 |
| 202 | 912.55 | 20.04 |
| 203 | 914.49 | 23.75 |
| 204 | 915.28 | 35.24 |
| 205 | 931.51 | 19.99 |
| 206 | 935.49 | 23.76 |
| 207 | 936.49 | 21.43 |
| 208 | 939.51 | 23.79 |
| 209 | 941.46 | 24.85 |
| 210 | 944.55 | 21.35 |
| 211 | 945.46 | 25.7 |
| 212 | 947.5 | 24.75 |
| 213 | 951.55 | 20.32 |
| 214 | 956.48 | 20.38 |
| 215 | 958.51 | 20.76 |
| 216 | 963.54 | 21.65 |
| 217 | 973.28 | 35.58 |
| 218 | 984.49 | 24.43 |
| 219 | 988.55 | 35.81 |
| 220 | 988.55 | 21.83 |
| 221 | 994.49 | 25.17 |
| 222 | 995.44 | 25.15 |
| 223 | 996.51 | 24.63 |
| 224 | 1009.47 | 20.96 |
| 225 | 1012.53 | 35.08 |
| 226 | 1016.31 | 35.67 |
| 227 | 1016.49 | 25.81 |
| 228 | 1027.55 | 24.6 |
| 229 | 1031.5 | 25.66 |
| 230 | 1037.57 | 21.98 |
| 231 | 1038.61 | 20.84 |
| 232 | 1042.54 | 25.13 |
| 233 | 1053.53 | 25.08 |
| 234 | 1056.57 | 25.49 |
| 235 | 1066.52 | 25.96 |
| 236 | 1068.56 | 21.69 |
| 237 | 1073.36 | 35.39 |
| 238 | 1078.69 | 19.67 |
| 239 | 1082.55 | 24.37 |
| 240 | 1083.56 | 21.65 |
| 241 | 1096.41 | 35.91 |
| 242 | 1096.53 | 26.12 |
| 243 | 1098.56 | 21.42 |
| 244 | 1099.56 | 21.56 |
| 245 | 1100.59 | 21.62 |
| 246 | 1101.54 | 22.21 |
| 247 | 1106.52 | 26.02 |
| 248 | 1109.68 | 21.06 |
| 249 | 1114.54 | 25.52 |
| 250 | 1114.57 | 21.27 |
| 251 | 1124.58 | 21.04 |
| 252 | 1125.55 | 24.9 |
| 253 | 1125.58 | 21.77 |
| 254 | 1126.52 | 21.2 |
| 255 | 1126.57 | 25.61 |
| 256 | 1134.63 | 23.68 |
| 257 | 1135.53 | 27.51 |
| 258 | 1136.59 | 20.27 |
| 259 | 1137.65 | 24.08 |
| 260 | 1138.64 | 19.55 |
| 261 | 1139.54 | 21.01 |
| 262 | 1140.53 | 21.14 |
| 263 | 1141.57 | 25.2 |
| 264 | 1142.61 | 21.8 |
| 265 | 1143.56 | 36.95 |
| 266 | 1144.43 | 35.57 |
| 267 | 1144.6 | 26.25 |
| 268 | 1150.61 | 22.42 |
| 269 | 1153.38 | 35.68 |
| 270 | 1153.57 | 26.34 |
| 271 | 1154.57 | 36.72 |
| 272 | 1155.55 | 20.77 |
| 273 | 1157.58 | 37.41 |
| 274 | 1159.63 | 20.75 |
| 275 | 1159.64 | 26.09 |
| 276 | 1160.6 | 26.14 |
| 277 | 1162.59 | 20.26 |
| 278 | 1165.55 | 37.15 |
| 279 | 1166.62 | 22.8 |
| 280 | 1168.58 | 26.23 |
| 281 | 1169.61 | 23.85 |
| 282 | 1170.64 | 21.61 |
| 283 | 1171.62 | 23.34 |
| 284 | 1173.58 | 37.51 |
| 285 | 1179.65 | 20.06 |
| 286 | 1181.62 | 26.06 |
| 287 | 1190.64 | 22.02 |
| 288 | 1196.37 | 36.14 |
| 289 | 1196.59 | 21.07 |
| 290 | 1198.6 | 26.05 |
| 291 | 1199.63 | 21.92 |
| 292 | 1200.59 | 24.21 |
| 293 | 1201.59 | 24.85 |
| 294 | 1204.65 | 21.93 |
| 295 | 1209.58 | 26.26 |
| 296 | 1210.45 | 36.58 |
| 297 | 1210.61 | 20.92 |
| 298 | 1213.62 | 27.75 |
| 299 | 1215.49 | 27.61 |
| 300 | 1216.59 | 24.27 |
| 301 | 1217.58 | 35.91 |
| 302 | 1218.42 | 36.88 |
| 303 | 1219.62 | 26.92 |
| 304 | 1220.63 | 22.11 |
| 305 | 1221.63 | 26.86 |
| 306 | 1223.6 | 27.47 |
| 307 | 1226.58 | 21 |
| 308 | 1226.63 | 23.47 |
| 309 | 1230.6 | 21.74 |
| 310 | 1234.42 | 36.16 |
| 311 | 1234.61 | 27.41 |
| 312 | 1238.55 | 36.74 |
| 313 | 1238.63 | 21.86 |
| 314 | 1239.52 | 34.91 |
| 315 | 1241.6 | 26.68 |
| 316 | 1245.6 | 21.63 |
| 317 | 1247.58 | 22.02 |
| 318 | 1250.52 | 35.35 |
| 319 | 1257.63 | 26.86 |
| 320 | 1260.61 | 21.84 |
| 321 | 1263.6 | 22.72 |
| 322 | 1267.64 | 21.55 |
| 323 | 1281.64 | 27.16 |
| 324 | 1284.62 | 28.22 |
| 325 | 1284.69 | 21.88 |
| 326 | 1287.65 | 21.85 |
| 327 | 1296.65 | 19.37 |
| 328 | 1300.65 | 23.07 |
| 329 | 1304.66 | 21.85 |
| 330 | 1309.74 | 21.97 |
| 331 | 1310.64 | 27.14 |
| 332 | 1321.63 | 37.98 |
| 333 | 1321.65 | 28.4 |
| 334 | 1328.64 | 26.87 |
| 335 | 1338.66 | 23.99 |
| 336 | 1340.65 | 28.33 |
| 337 | 1349.48 | 36.47 |
| 338 | 1349.77 | 20.22 |
| 339 | 1350.65 | 21.56 |
| 340 | 1352.62 | 29.68 |
| 341 | 1352.83 | 24.47 |
| 342 | 1353.65 | 21.48 |
| 343 | 1358.44 | 36.46 |
| 344 | 1361.69 | 21.95 |
| 345 | 1362.72 | 22.13 |
| 346 | 1366.68 | 27.48 |
| 347 | 1368.68 | 21.7 |
| 348 | 1375.67 | 23.28 |
| 349 | 1378.67 | 28.85 |
| 350 | 1383.69 | 39.02 |
| 351 | 1390.5 | 37.05 |
| 352 | 1392.68 | 21.75 |
| 353 | 1395.71 | 22.59 |
| 354 | 1396.5 | 37.63 |
| 355 | 1396.68 | 28.05 |
| 356 | 1413.62 | 25.78 |
| 357 | 1417.69 | 20.01 |
| 358 | 1421.58 | 37.51 |
| 359 | 1423.68 | 21.47 |
| 360 | 1425.65 | 22.34 |
| 361 | 1436.79 | 22.82 |
| 362 | 1438.72 | 21.98 |
| 363 | 1439.72 | 29.7 |
| 364 | 1440.72 | 39.19 |
| 365 | 1440.75 | 21.63 |
| 366 | 1442.68 | 27.76 |
| 367 | 1444.49 | 36.69 |
| 368 | 1448.83 | 20.53 |
| 369 | 1449.7 | 21.84 |
| 370 | 1451.71 | 29.19 |
| 371 | 1452.72 | 23.55 |
| 372 | 1456.75 | 22.48 |
| 373 | 1457.68 | 22 |
| 374 | 1458.69 | 28.03 |
| 375 | 1460.71 | 20.31 |
| 376 | 1473.7 | 22.3 |
| 377 | 1474.75 | 19.94 |
| 378 | 1483.72 | 22.59 |
| 379 | 1487.71 | 29.58 |
| 380 | 1491.8 | 39.86 |
| 381 | 1493.74 | 22.11 |
| 382 | 1507.8 | 39.98 |
| 383 | 1508.77 | 23.17 |
| 384 | 1509.59 | 37.07 |
| 385 | 1510.72 | 28.39 |
| 386 | 1510.75 | 20.11 |
| 387 | 1522.78 | 22.81 |
| 388 | 1523.89 | 29.75 |
| 389 | 1524.72 | 20.02 |
| 390 | 1535.75 | 22.03 |
| 391 | 1540.81 | 39.97 |
| 392 | 1549.76 | 39.5 |
| 393 | 1552.57 | 37.35 |
| 394 | 1556.8 | 40.03 |
| 395 | 1560.69 | 22.08 |
| 396 | 1562.76 | 22.48 |
| 397 | 1563.77 | 29.56 |
| 398 | 1564.78 | 28.53 |
| 399 | 1567.77 | 20.2 |
| 400 | 1576.74 | 46.02 |
| 401 | 1577.75 | 40.06 |
| 402 | 1579.75 | 20.05 |
| 403 | 1579.78 | 29.83 |
| 404 | 1609.81 | 30.25 |
| 405 | 1610.91 | 30.12 |
| 406 | 1611.83 | 40.12 |
| 407 | 1611.89 | 23.27 |
| 408 | 1613.89 | 23.98 |
| 409 | 1619.78 | 22.6 |
| 410 | 1620.74 | 29.58 |
| 411 | 1620.79 | 22.71 |
| 412 | 1624.61 | 37.73 |
| 413 | 1631.78 | 47.02 |
| 414 | 1632.79 | 38.03 |
| 415 | 1633.83 | 19.58 |
| 416 | 1635.83 | 30.38 |
| 417 | 1636.79 | 20.1 |
| 418 | 1636.81 | 30.33 |
| 419 | 1638.8 | 20.24 |
| 420 | 1640.75 | 28.28 |
| 421 | 1658.68 | 21.55 |
| 422 | 1658.79 | 30.04 |
| 423 | 1660.76 | 37.7 |
| 424 | 1664.82 | 29.87 |
| 425 | 1668.87 | 40.47 |
| 426 | 1673.82 | 29.4 |
| 427 | 1674.83 | 20.71 |
| 428 | 1680.82 | 30.02 |
| 429 | 1684.74 | 31.42 |
| 430 | 1684.81 | 39.43 |
| 431 | 1689.86 | 27.62 |
| 432 | 1693.83 | 20.48 |
| 433 | 1693.84 | 23.48 |
| 434 | 1697.81 | 30.86 |
| 435 | 1698.67 | 37.53 |
| 436 | 1705.78 | 46.77 |
| 437 | 1708.85 | 31.08 |
| 438 | 1725.66 | 38.3 |
| 439 | 1728.84 | 36.74 |
| 440 | 1729.6 | 53.46 |
| 441 | 1737.85 | 31.04 |
| 442 | 1746.95 | 19.75 |
| 443 | 1749.88 | 30.61 |
| 444 | 1761.91 | 21.61 |
| 445 | 1764.86 | 29.97 |
| 446 | 1767.07 | 24.12 |
| 447 | 1768.9 | 20.78 |
| 448 | 1769.78 | 28.25 |
| 449 | 1773.87 | 34.89 |
| 450 | 1782.91 | 25.93 |
| 451 | 1783.86 | 39.94 |
| 452 | 1784.88 | 40.04 |
| 453 | 1786.66 | 38.34 |
| 454 | 1788.89 | 30.26 |
| 455 | 1794.84 | 30.49 |
| 456 | 1796.85 | 30.86 |
| 457 | 1798.79 | 37.33 |
| 458 | 1807.91 | 20.7 |
| 459 | 1813.78 | 31.79 |
| 460 | 1818.9 | 31.1 |
| 461 | 1821.88 | 30.43 |
| 462 | 1823.99 | 24.61 |
| 463 | 1825.87 | 31.81 |
| 464 | 1829.03 | 21.24 |
| 465 | 1834.9 | 31.15 |
| 466 | 1835.92 | 24.04 |
| 467 | 1838.89 | 27.03 |
| 468 | 1846.93 | 32.08 |
| 469 | 1852.96 | 25.14 |
| 470 | 1858.91 | 24.21 |
| 471 | 1859.9 | 24.38 |
| 472 | 1860.42 | 33.82 |
| 473 | 1863.95 | 44.05 |
| 474 | 1880.97 | 44.03 |
| 475 | 1885.96 | 21.26 |
| 476 | 1889.94 | 33.12 |
| 477 | 1891.65 | 54.28 |
| 478 | 1892.95 | 22.4 |
| 479 | 1893.06 | 24.64 |
| 480 | 1899.93 | 21.64 |
| 481 | 1911.05 | 30.74 |
| 482 | 1912.74 | 32.7 |
| 483 | 1916.85 | 20.35 |
| 484 | 1926.91 | 30.99 |
| 485 | 1927.99 | 19.53 |
| 486 | 1932.98 | 21.65 |
| 487 | 1933.95 | 21.63 |
| 488 | 1936.11 | 20.51 |
| 489 | 1938.96 | 21.33 |
| 490 | 1942.77 | 36.19 |
| 491 | 1945.07 | 33.71 |
| 492 | 1946.95 | 31.75 |
| 493 | 1949.97 | 21.66 |
| 494 | 1952.42 | 25.65 |
| 495 | 1955.04 | 25.29 |
| 496 | 1955.94 | 28.14 |
| 497 | 1969.43 | 32.31 |
| 498 | 1976.96 | 32.36 |
| 499 | 1977.99 | 32.21 |
| 500 | 1989.02 | 24.75 |
| 501 | 1992.02 | 22.05 |
| 502 | 1993.02 | 27.07 |
| 503 | 1993.96 | 32.2 |
| 504 | 1997.99 | 25.13 |
| 505 | 2004.02 | 24.63 |
| 506 | 2006.93 | 33 |
| 507 | 2008.03 | 22.13 |
| 508 | 2021.06 | 21.98 |
| 509 | 2022.97 | 33.39 |
| 510 | 2023.99 | 21.47 |
| 511 | 2026.1 | 25.66 |
| 512 | 2031 | 21.8 |
| 513 | 2035.07 | 40.19 |
| 514 | 2040.02 | 32.87 |
| 515 | 2042.14 | 25.21 |
| 516 | 2057.04 | 24.85 |
| 517 | 2065 | 24.48 |
| 518 | 2076.61 | 32.44 |
| 519 | 2078.05 | 22.47 |
| 520 | 2081.03 | 20.17 |
| 521 | 2087.96 | 19.46 |
| 522 | 2088.04 | 32.92 |
| 523 | 2088.95 | 23.68 |
| 524 | 2090.04 | 39.48 |
| 525 | 2095.53 | 24.83 |
| 526 | 2100.12 | 19.6 |
| 527 | 2110 | 24.11 |
| 528 | 2114.07 | 31.61 |
| 529 | 2118.04 | 27.72 |
| 530 | 2129.06 | 26.97 |
| 531 | 2134.04 | 27.78 |
| 532 | 2138.03 | 21.78 |
| 533 | 2146 | 52.45 |
| 534 | 2188.03 | 39.71 |
| 535 | 2192.08 | 22.16 |
| 536 | 2195.49 | 33.36 |
| 537 | 2197.94 | 34.23 |
| 538 | 2199.08 | 22.31 |
| 539 | 2207.16 | 43.61 |
| 540 | 2208 | 32.37 |
| 541 | 2208.97 | 31.75 |
| 542 | 2211.11 | 20.28 |
| 543 | 2212.06 | 33.53 |
| 544 | 2219.56 | 33.59 |
| 545 | 2227.05 | 33.42 |
| 546 | 2233.1 | 22.49 |
| 547 | 2237.06 | 27.1 |
| 548 | 2244.17 | 19.48 |
| 549 | 2248.75 | 22.52 |
| 550 | 2251.77 | 33.11 |
| 551 | 2264.12 | 22.67 |
| 552 | 2269.29 | 38.14 |
| 553 | 2279.57 | 23.77 |
| 554 | 2282.13 | 22.29 |
| 555 | 2283.12 | 26.51 |
| 556 | 2292.21 | 21.29 |
| 557 | 2302.38 | 26.16 |
| 558 | 2305.7 | 34.8 |
| 559 | 2310.15 | 41.24 |
| 560 | 2315.22 | 19.45 |
| 561 | 2318.29 | 26.28 |
| 562 | 2319.19 | 33.87 |
| 563 | 2320.79 | 22.48 |
| 564 | 2322.17 | 22.28 |
| 565 | 2323.14 | 22.39 |
| 566 | 2328.06 | 20.83 |
| 567 | 2332.08 | 50.2 |
| 568 | 2335.75 | 26.61 |
| 569 | 2339.08 | 34.01 |
| 570 | 2339.17 | 22.63 |
| 571 | 2343.49 | 20.08 |
| 572 | 2361.19 | 20.78 |
| 573 | 2367.48 | 34.22 |
| 574 | 2371.18 | 22.77 |
| 575 | 2377.19 | 20.79 |
| 576 | 2380.16 | 36.49 |
| 577 | 2389.33 | 22.4 |
| 578 | 2392.8 | 35.28 |
| 579 | 2394 | 23.93 |
| 580 | 2405.6 | 22.19 |
| 581 | 2410.16 | 23.45 |
| 582 | 2414.26 | 19.58 |
| 583 | 2430.19 | 28.28 |
| 584 | 2430.72 | 35.39 |
| 585 | 2436.15 | 22.8 |
| 586 | 2437.22 | 22.86 |
| 587 | 2445.8 | 28.15 |
| 588 | 2451.59 | 24.02 |
| 589 | 2471.25 | 34.76 |
| 590 | 2478.22 | 22.45 |
| 591 | 2479.15 | 48.79 |
| 592 | 2485.16 | 34.39 |
| 593 | 2501.21 | 34.37 |
| 594 | 2502.73 | 22.84 |
| 595 | 2507.21 | 22.78 |
| 596 | 2518.4 | 22.8 |
| 597 | 2525.21 | 27.72 |
| 598 | 2534.56 | 22.81 |
| 599 | 2548.38 | 35.1 |
| 600 | 2551.32 | 34.75 |
| 601 | 2553.52 | 34.27 |
| 602 | 2557.15 | 28.17 |
| 603 | 2559.25 | 19.44 |
| 604 | 2565.18 | 23.74 |
| 605 | 2567.28 | 28.17 |
| 606 | 2570.28 | 42.66 |
| 607 | 2574.08 | 32.92 |
| 608 | 2580.19 | 23 |
| 609 | 2587.39 | 21.17 |
| 610 | 2599.26 | 28.18 |
| 611 | 2607.6 | 34.4 |
| 612 | 2616 | 28.34 |
| 613 | 2627.08 | 23.44 |
| 614 | 2632.18 | 24.96 |
| 615 | 2639.41 | 20.98 |
| 616 | 2668.46 | 42.02 |
| 617 | 2682.22 | 22.49 |
| 618 | 2702.1 | 38.01 |
| 619 | 2710.58 | 35.15 |
| 620 | 2723.92 | 23.81 |
| 621 | 2726.32 | 28.87 |
| 622 | 2726.37 | 42.94 |
| 623 | 2733.84 | 34.18 |
| 624 | 2742.36 | 42.12 |
| 625 | 2748.87 | 36.35 |
| 626 | 2754.36 | 29.64 |
| 627 | 2761.4 | 21.49 |
| 628 | 2777.61 | 21.54 |
| 629 | 2796.02 | 21 |
| 630 | 2810.46 | 36.66 |
| 631 | 2816.5 | 28.41 |
| 632 | 2821.19 | 23.73 |
| 633 | 2837.91 | 23.99 |
| 634 | 2839.42 | 24.17 |
| 635 | 2853.32 | 23.8 |
| 636 | 2861.32 | 50.73 |
| 637 | 2887.42 | 35.66 |
| 638 | 2889.19 | 24.09 |
| 639 | 2889.65 | 28.73 |
| 640 | 2903.72 | 35.61 |
| 641 | 2911 | 21.68 |
| 642 | 2914.57 | 24.28 |
| 643 | 2917.41 | 29.03 |
| 644 | 2924.58 | 22.97 |
| 645 | 2926.2 | 22.16 |
| 646 | 2932.36 | 34.11 |
| 647 | 2942.4 | 22.22 |
| 648 | 2977.61 | 29.14 |
| 649 | 2982.9 | 22.24 |
| 650 | 2989.4 | 24.42 |
| 651 | 2999.23 | 22.22 |
| 652 | 3002.47 | 23.95 |
| 653 | 3011.46 | 29.72 |
| 654 | 3013.27 | 22.28 |
| 655 | 3017.95 | 49.05 |
| 656 | 3023.33 | 24.55 |
| 657 | 3025.69 | 29.85 |
| 658 | 3035 | 42.23 |
| 659 | 3035.17 | 47.18 |
| 660 | 3038.11 | 22.42 |
| 661 | 3041.47 | 29.95 |
| 662 | 3044.98 | 34.34 |
| 663 | 3047.64 | 26.68 |
| 664 | 3056.94 | 45.28 |
| 665 | 3064.36 | 20.55 |
| 666 | 3076.33 | 19.64 |
| 667 | 3080.99 | 29.92 |
| 668 | 3091.41 | 28.33 |
| 669 | 3092.45 | 30.97 |
| 670 | 3092.51 | 36.23 |
| 671 | 3098.49 | 30.07 |
| 672 | 3112.42 | 22.44 |
| 673 | 3113.81 | 20.39 |
| 674 | 3114.66 | 30.3 |
| 675 | 3121.31 | 30.23 |
| 676 | 3129.86 | 35.93 |
| 677 | 3136.55 | 24.5 |
| 678 | 3139.44 | 29.48 |
| 679 | 3143.51 | 32.87 |
| 680 | 3145.49 | 38.8 |
| 681 | 3152.23 | 24.54 |
| 682 | 3158.6 | 29.69 |
| 683 | 3168.38 | 24.69 |
| 684 | 3178.26 | 30.25 |
| 685 | 3184.67 | 24.91 |
| 686 | 3193.27 | 22.63 |
| 687 | 3195.2 | 20.64 |
| 688 | 3202.2 | 30.52 |
| 689 | 3204.32 | 35.31 |
| 690 | 3219.43 | 35.05 |
| 691 | 3223.42 | 24.74 |
| 692 | 3240.47 | 25.2 |
| 693 | 3249.16 | 22.61 |
| 694 | 3258.51 | 22.89 |
| 695 | 3260.4 | 41.55 |
| 696 | 3261.35 | 22.17 |
| 697 | 3265.33 | 36.02 |
| 698 | 3271.39 | 30.75 |
| 699 | 3276.25 | 33.59 |
| 700 | 3282.79 | 30.09 |
| 701 | 3287.39 | 30.84 |
| 702 | 3290.39 | 24.1 |
| 703 | 3295.51 | 25.38 |
| 704 | 3298.48 | 36.06 |
| 705 | 3310.26 | 36.5 |
| 706 | 3314.41 | 20.12 |
| 707 | 3318.68 | 30.98 |
| 708 | 3325.9 | 54.11 |
| 709 | 3333.28 | 19.42 |
| 710 | 3333.8 | 23.85 |
| 711 | 3334.27 | 31 |
| 712 | 3336.48 | 39.43 |
| 713 | 3341.91 | 21.05 |
| 714 | 3356.38 | 25.56 |
| 715 | 3359.67 | 31.88 |
| 716 | 3363.69 | 30.21 |
| 717 | 3375.69 | 31.9 |
| 718 | 3386.16 | 22.7 |
| 719 | 3400.07 | 42.03 |
| 720 | 3416.82 | 36.78 |
| 721 | 3435.94 | 22.7 |
| 722 | 3457.46 | 35.83 |
| 723 | 3457.73 | 31.51 |
| 724 | 3472.36 | 21.75 |
| 725 | 3510.65 | 40.23 |
| 726 | 3521.07 | 30.74 |
| 727 | 3530.63 | 26.11 |
| 728 | 3531.46 | 30.16 |
| 729 | 3546.82 | 26.7 |
| 730 | 3559.71 | 24.89 |
| 731 | 3571.61 | 31.03 |
| 732 | 3582.7 | 19.47 |
| 733 | 3583.5 | 41.45 |
| 734 | 3590.72 | 29.01 |
| 735 | 3596.51 | 21.57 |
| 736 | 3603.92 | 20.21 |
| 737 | 3633.02 | 33.15 |
| 738 | 3633.71 | 27.02 |
| 739 | 3635.47 | 31.87 |
| 740 | 3669.71 | 24.13 |
| 741 | 3686.04 | 22.21 |
| 742 | 3696.88 | 26.94 |
| 743 | 3702.45 | 32.45 |
| 744 | 3703.85 | 22.51 |
| 745 | 3706.68 | 22 |
| 746 | 3710.86 | 19.6 |
| 747 | 3737.71 | 37.15 |
| 748 | 3738.58 | 24.74 |
| 749 | 3765.51 | 20.21 |
| 750 | 3774.84 | 22.9 |
| 751 | 3775.25 | 27 |
| 752 | 3775.9 | 25.28 |
| 753 | 3790.98 | 32.88 |
| 754 | 3816.45 | 21.94 |
| 755 | 3839.91 | 19.7 |
| 756 | 3864.66 | 33.71 |
| 757 | 3906.75 | 24.13 |
| 758 | 3935.56 | 34.14 |
| 759 | 3948.83 | 45.72 |
| 760 | 3968.68 | 21.11 |
| 761 | 4002.73 | 20.66 |
| 762 | 4008.95 | 23.57 |
| 763 | 4025.43 | 33.23 |
| 764 | 4042.93 | 33.13 |
| 765 | 4044.98 | 26.39 |
| 766 | 4069.65 | 25.2 |
| 767 | 4081.58 | 24.53 |
| 768 | 4083.75 | 31.32 |
| 769 | 4097.98 | 24.6 |
| 770 | 4113.77 | 24.46 |
| 771 | 4122.1 | 24.06 |
| 772 | 4124.05 | 21.49 |
| 773 | 4143.81 | 26.55 |
| 774 | 4196.47 | 20.96 |
| 775 | 4215.52 | 26.95 |
| 776 | 4217.98 | 26 |
| 777 | 4229.11 | 29.06 |
| 778 | 4278.77 | 23.34 |
| 779 | 4306 | 25.13 |
| 780 | 4322.14 | 25.18 |
| 781 | 4339 | 25.4 |
| 782 | 4352.98 | 20.17 |
| 783 | 4363.14 | 23.05 |
| 784 | 4369.07 | 20.21 |
| 785 | 4378.88 | 23.14 |
| 786 | 4381.08 | 34.13 |
| 787 | 4395.06 | 23.09 |
| 788 | 4415.72 | 22.53 |
| 789 | 4426.27 | 20.09 |
| 790 | 4436.04 | 26.25 |
| 791 | 4441.22 | 22.43 |
| 792 | 4483.33 | 25.4 |
| 793 | 4491.91 | 26.23 |
| 794 | 4538.61 | 26.35 |
| 795 | 4548.93 | 26.45 |
| 796 | 4550.95 | 23.31 |
| 797 | 4567.27 | 23.29 |
| 798 | 4583.7 | 35.76 |
| 799 | 4619.43 | 25.07 |
| 800 | 4637.97 | 25.75 |
| 801 | 4805.62 | 26.47 |
| 802 | 4817.33 | 23.86 |
| 803 | 4833.29 | 23.89 |
| 804 | 4849.07 | 23.86 |
| 805 | 4863.23 | 26.67 |
| 806 | 4864.81 | 23.88 |
| 807 | 4933.46 | 20.58 |
| 808 | 4976.46 | 20.62 |
| 809 | 5027.18 | 26.53 |
| 810 | 5039.24 | 25.56 |
| 811 | 5043.12 | 26.54 |
| 812 | 5090.45 | 20.1 |
| 813 | 5160.13 | 21.74 |
| 814 | 5229.59 | 25.1 |
| 815 | 5276.59 | 20.04 |
| 816 | 5495.5 | 19.52 |
| 817 | 5574.44 | 23.22 |
| 818 | 6186.07 | 24.91 |
| 819 | 6237.1 | 21.07 |
| 820 | 6491.14 | 19.44 |
| 821 | 6650.84 | 25.52 |
| 822 | 7907.23 | 19.59 |
| 823 | 7958.72 | 34.38 |
| 824 | 8289.54 | 19.54 |
| 825 | 8445.41 | 19.52 |
| 826 | 8837.64 | 21.06 |
| 827 | 8853.9 | 21.1 |
| 828 | 8917.52 | 22.55 |
| 829 | 10199.94 | 21.11 |
| 830 | 10753.72 | 19.71 |
| 831 | 10770.19 | 19.64 |
| 832 | 11041.95 | 21.98 |
| 833 | 11738.36 | 19.6 |
| 834 | 11967.98 | 20.48 |
| 835 | 14111.34 | 21.97 |
| 836 | 3266.45 | 22.73 |

20. The method according to claim 1, wherein an evaluation of the determined presence or absence of markers 1 to 149 is effected by using the following reference values:

| | Occurrence in | Occurrence in |
|---|---|---|
| Number | group BC | Control |
| 1 | 0.86 | 0.13 |
| 2 | 0.71 | 0 |
| 3 | 0.73 | 0.03 |
| 4 | 0.71 | 0.07 |
| 5 | 0.64 | 0.03 |
| 6 | 0.64 | 0.03 |
| 7 | 0.71 | 0.13 |
| 8 | 0.71 | 0.13 |
| 9 | 0.73 | 0.16 |
| 10 | 0.67 | 0.09 |
| 11 | 0.57 | 0 |
| 12 | 0.92 | 0.35 |
| 13 | 0.57 | 0 |
| 14 | 0.57 | 0 |
| 15 | 0.57 | 0.03 |
| 16 | 0.57 | 0.03 |
| 17 | 0.57 | 0.03 |
| 18 | 0.57 | 0.03 |
| 19 | 0.57 | 0.03 |
| 20 | 0.67 | 0.13 |
| 21 | 0.78 | 0.25 |
| 22 | 0.69 | 0.16 |
| 23 | 0.78 | 0.25 |
| 24 | 0.69 | 0.17 |
| 25 | 0.56 | 0.03 |
| 26 | 0.57 | 0.05 |
| 27 | 0.67 | 0.16 |
| 28 | 0.6 | 0.09 |
| 29 | 0.51 | 0 |
| 30 | 0.86 | 0.35 |
| 31 | 0.91 | 0.41 |
| 32 | 0.71 | 0.22 |
| 33 | 0.51 | 0.03 |
| 34 | 0.58 | 0.09 |
| 35 | 0.89 | 0.41 |
| 36 | 0.51 | 0.03 |
| 37 | 0.73 | 0.25 |
| 38 | 0.51 | 0.03 |
| 39 | 0.62 | 0.16 |
| 40 | 0.69 | 0.22 |
| 41 | 0.58 | 0.13 |
| 42 | 0.58 | 0.13 |
| 43 | 0.73 | 0.38 |
| 44 | 0.8 | 0.5 |
| 45 | 0.42 | 0.72 |
| 46 | 0.51 | 0.81 |
| 47 | 0.44 | 0.75 |
| 48 | 0.62 | 0.95 |
| 49 | 0.57 | 0.9 |
| 50 | 0.57 | 0.93 |
| 51 | 0.29 | 0.65 |
| 52 | 0.29 | 0.68 |
| 53 | 0.42 | 0.81 |
| 54 | 0.57 | 0.97 |
| 55 | 0.14 | 0.55 |
| 56 | 0.31 | 0.78 |
| 57 | 0.33 | 0.81 |
| 58 | 0 | 0.5 |
| 59 | 0 | 0.5 |
| 60 | 0 | 0.5 |
| 61 | 0 | 0.5 |
| 62 | 0 | 0.5 |
| 63 | 0 | 0.5 |
| 64 | 0 | 0.5 |
| 65 | 0 | 0.5 |
| 66 | 0.29 | 0.8 |
| 67 | 0.14 | 0.65 |
| 68 | 0.14 | 0.65 |
| 69 | 0.14 | 0.65 |
| 70 | 0.29 | 0.8 |
| 71 | 0.29 | 0.8 |
| 72 | 0.14 | 0.65 |
| 73 | 0.14 | 0.65 |
| 74 | 0.29 | 0.8 |
| 75 | 0.18 | 0.7 |
| 76 | 0.33 | 0.85 |
| 77 | 0.08 | 0.6 |
| 78 | 0.43 | 0.95 |
| 79 | 0.43 | 0.95 |
| 80 | 0.31 | 0.82 |
| 81 | 0.2 | 0.72 |
| 82 | 0.14 | 0.68 |
| 83 | 0.14 | 0.68 |
| 84 | 0.14 | 0.68 |
| 85 | 0.14 | 0.68 |
| 86 | 0 | 0.53 |
| 87 | 0 | 0.53 |
| 88 | 0.29 | 0.82 |
| 89 | 0.29 | 0.82 |
| 90 | 0.29 | 0.82 |
| 91 | 0.29 | 0.82 |
| 92 | 0.29 | 0.82 |
| 93 | 0.29 | 0.82 |
| 94 | 0.29 | 0.82 |
| 95 | 0.26 | 0.8 |
| 96 | 0.43 | 0.97 |
| 97 | 0.43 | 0.97 |
| 98 | 0.43 | 0.97 |
| 99 | 0.29 | 0.85 |
| 100 | 0.14 | 0.7 |
| 101 | 0.29 | 0.85 |
| 102 | 0.14 | 0.7 |
| 103 | 0.29 | 0.85 |
| 104 | 0.29 | 0.85 |
| 105 | 0.29 | 0.85 |
| 106 | 0.26 | 0.82 |
| 107 | 0 | 0.57 |
| 108 | 0.26 | 0.82 |
| 109 | 0 | 0.57 |
| 110 | 0 | 0.57 |
| 111 | 0 | 0.57 |
| 112 | 0 | 0.57 |
| 113 | 0.43 | 1 |
| 114 | 0.24 | 0.81 |
| 115 | 0.14 | 0.72 |
| 116 | 0.14 | 0.72 |
| 117 | 0.14 | 0.72 |
| 118 | 0.29 | 0.88 |
| 119 | 0.29 | 0.88 |
| 120 | 0.29 | 0.88 |
| 121 | 0.36 | 0.95 |
| 122 | 0 | 0.6 |
| 123 | 0 | 0.6 |
| 124 | 0 | 0.6 |
| 125 | 0 | 0.6 |
| 126 | 0.29 | 0.9 |
| 127 | 0.14 | 0.75 |
| 128 | 0.29 | 0.9 |
| 129 | 0.18 | 0.8 |
| 130 | 0.13 | 0.75 |
| 131 | 0.31 | 0.93 |
| 132 | 0.14 | 0.78 |
| 133 | 0 | 0.63 |
| 134 | 0.28 | 0.93 |
| 135 | 0 | 0.65 |
| 136 | 0.18 | 0.82 |
| 137 | 0.14 | 0.8 |
| 138 | 0.14 | 0.8 |
| 139 | 0 | 0.68 |
| 140 | 0 | 0.68 |
| 141 | 0 | 0.7 |
| 142 | 0 | 0.7 |
| 143 | 0 | 0.75 |
| 144 | 0 | 0.75 |
| 145 | 0.14 | 0.95 |
| 146 | 0.15 | 0.55 |
| 147 | 0.15 | 0.6 |
| 148 | 0.15 | 0.57 |
| 149 | 0.15 | 0.56 |

21. The method according to claim 1, wherein an evaluation of the amplitude of markers 150 to 185 is effected by using the following reference values:

| | Mean amplitude | Mean amplitude |
|---|---|---|
| | in group BC | in control |
| Number | [ppm] | samples [ppm] |
| 150 | 512 | 234 |
| 151 | 4341 | 11833 |
| 152 | 320 | 107 |
| 153 | 4943 | 19582 |
| 154 | 831 | 404 |
| 155 | 1615 | 4183 |
| 156 | 882 | 2816 |
| 157 | 532 | 573 |
| 158 | 922 | 2703 |
| 159 | 3070 | 7231 |
| 160 | 1863 | 3755 |
| 161 | 2322 | 5187 |
| 162 | 6370 | 1431 |
| 163 | 1394 | 580 |
| 164 | 1039 | 428 |
| 165 | 225 | 542 |
| 166 | 769 | 1578 |
| 167 | 42329 | 112162 |
| 168 | 2602 | 981 |
| 169 | 321 | 745 |
| 170 | 106 | 308 |
| 171 | 596 | 1337 |
| 172 | 4128 | 10960 |
| 173 | 761 | 1961 |
| 174 | 200 | 428 |
| 175 | 2150 | 4372 |
| 176 | 995 | 2026 |
| 177 | 215 | 654 |
| 178 | 215 | 449 |
| 179 | 281 | 574 |
| 180 | 691 | 1578 |
| 181 | 1860 | 4502 |
| 182 | 1078 | 6650 |
| 183 | 406 | 846 |
| 184 | 342 | 1360 |
| 185 | 1425 | 10166 |

22. The method according to claim 1, wherein an evaluation of the amplitude of markers 186 to 836 is effected by using the following reference values:

| | Mean | Mean |
|---|---|---|
| | amplitude | amplitude |
| | in group | in |
| Number | BC | Controls |
| 186 | 119.63 | 80.79 |
| 187 | 77.4 | 56.61 |
| 188 | 44.83 | 19.49 |
| 189 | 75.44 | 45.2 |
| 190 | 118.21 | 235.42 |
| 191 | 74.62 | 33.56 |
| 192 | 374.91 | 44.29 |
| 193 | 157.08 | 123.78 |
| 194 | 453.57 | 10.31 |
| 195 | 88 | 45.2 |
| 196 | 90.46 | 720.98 |
| 197 | 111.95 | 85.68 |
| 198 | 410.43 | 469.76 |
| 199 | 427.05 | 319.41 |
| 200 | 83.12 | 93.94 |
| 201 | 102.07 | 57.89 |
| 202 | 238.89 | 119.03 |
| 203 | 111.31 | 90.37 |
| 204 | 195.66 | 197.97 |
| 205 | 223.5 | 223.37 |
| 206 | 149.65 | 149.75 |
| 207 | 159.86 | 56.01 |
| 208 | 353.83 | 70.68 |
| 209 | 79.65 | 40.47 |
| 210 | 669.1 | 617.85 |
| 211 | 153.61 | 135.8 |
| 212 | 163.45 | 287.56 |
| 213 | 1141.91 | 27.72 |
| 214 | 216.39 | 127.03 |
| 215 | 833.58 | 39.12 |
| 216 | 139 | 88.24 |
| 217 | 397.21 | 292.45 |
| 218 | 126.14 | 93.76 |
| 219 | 421.86 | 309.18 |
| 220 | 136.07 | 221.65 |
| 221 | 152.19 | 120.89 |
| 222 | 219.55 | 145.69 |
| 223 | 125.21 | 31.02 |
| 224 | 84.53 | 308.11 |
| 225 | 83.05 | 172.95 |
| 226 | 1842.39 | 1141.1 |
| 227 | 1324.02 | 1031.5 |
| 228 | 350.65 | 89.47 |
| 229 | 170.43 | 64.39 |
| 230 | 190.08 | 78.16 |
| 231 | 489.23 | 26.8 |
| 232 | 1996.47 | 72.63 |
| 233 | 188.24 | 95.18 |
| 234 | 161.17 | 119.99 |
| 235 | 103.9 | 97.75 |
| 236 | 480.36 | 237.07 |
| 237 | 5707.2 | 7336.8 |
| 238 | 379.06 | 56.43 |
| 239 | 308.98 | 301.08 |
| 240 | 462.23 | 78.2 |
| 241 | 194.93 | 151.59 |
| 242 | 5055.93 | 4175.1 |
| 243 | 366.14 | 189.83 |
| 244 | 367.44 | 170.6 |
| 245 | 224.51 | 141.51 |
| 246 | 183.13 | 124.78 |
| 247 | 106.82 | 88.51 |
| 248 | 3078.26 | 140.75 |
| 249 | 3190.15 | 3178.22 |
| 250 | 368.81 | 72.76 |
| 251 | 256.09 | 245.38 |
| 252 | 130.05 | 134.05 |
| 253 | 448.49 | 239.5 |
| 254 | 232.48 | 315.01 |
| 255 | 205.39 | 158.14 |
| 256 | 404.85 | 342.62 |
| 257 | 249.07 | 176.66 |
| 258 | 94.05 | 45.1 |
| 259 | 175.28 | 119.61 |
| 260 | 202.45 | 124.75 |
| 261 | 853.59 | 1140.19 |
| 262 | 1327.35 | 1993.54 |
| 263 | 356.82 | 284.59 |
| 264 | 265.05 | 150.92 |
| 265 | 558.75 | 451.29 |
| 266 | 221.77 | 143.72 |
| 267 | 1128.53 | 72.92 |
| 268 | 331.84 | 200.42 |
| 269 | 505.65 | 421.74 |
| 270 | 261.75 | 225.98 |
| 271 | 169.57 | 130.21 |
| 272 | 266.62 | 223.68 |
| 273 | 1362.12 | 1707.78 |
| 274 | 294.36 | 402.59 |
| 275 | 221.55 | 166.84 |
| 276 | 335.58 | 44.14 |
| 277 | 259.95 | 223.47 |
| 278 | 768.74 | 684.34 |
| 279 | 251.7 | 214.42 |
| 280 | 169.01 | 89.8 |
| 281 | 206.44 | 145.14 |
| 282 | 3791.23 | 83.4 |
| 283 | 147.66 | 91.52 |
| 284 | 257.97 | 474.54 |
| 285 | 2312.84 | 72.28 |
| 286 | 2355.55 | 150.4 |
| 287 | 1856.18 | 0 |
| 288 | 22744.1 | 27845.6 |
| 289 | 579.96 | 1024.9 |
| 290 | 181 | 79.4 |
| 291 | 449.65 | 287.08 |
| 292 | 8039.6 | 6153.2 |
| 293 | 400.81 | 327.89 |
| 294 | 310.29 | 151 |
| 295 | 1031.54 | 860.46 |
| 296 | 633.16 | 564.48 |
| 297 | 218.71 | 298.42 |
| 298 | 687.69 | 58.53 |
| 299 | 462.1 | 597.81 |
| 300 | 1581.02 | 1215.02 |
| 301 | 2630.29 | 3679.53 |
| 302 | 979.29 | 1913.27 |
| 303 | 243.92 | 96.75 |
| 304 | 173.39 | 55.41 |
| 305 | 148.48 | 73.15 |
| 306 | 156.84 | 179.86 |
| 307 | 469.03 | 595.19 |
| 308 | 133.11 | 102.43 |
| 309 | 792.94 | 497.12 |
| 310 | 1537.92 | 2939.46 |
| 311 | 1580.14 | 1795.44 |
| 312 | 193.48 | 190.55 |
| 313 | 794.93 | 201.72 |
| 314 | 928.42 | 699.24 |
| 315 | 143.26 | 115.01 |
| 316 | 303.03 | 129.16 |
| 317 | 1342.16 | 932.91 |
| 318 | 1790.3 | 3379.16 |
| 319 | 746.14 | 204.66 |
| 320 | 706.29 | 396.08 |
| 321 | 609.39 | 342.54 |
| 322 | 201.07 | 279.53 |
| 323 | 440.59 | 252.99 |
| 324 | 1500.12 | 166.99 |
| 325 | 231.13 | 194.08 |
| 326 | 690.08 | 362.46 |
| 327 | 239.27 | 204.76 |
| 328 | 312.93 | 156.8 |
| 329 | 325.22 | 238 |
| 330 | 8160.78 | 334.35 |
| 331 | 271 | 198.75 |
| 332 | 543.53 | 962.52 |
| 333 | 10101.8 | 12695.3 |
| 334 | 459.58 | 129.16 |
| 335 | 648.02 | 415.43 |
| 336 | 163.79 | 121.63 |
| 337 | 205.35 | 258.72 |
| 338 | 4607.6 | 550.93 |
| 339 | 112.46 | 181.81 |
| 340 | 1168.83 | 221.78 |
| 341 | 407.83 | 425.82 |
| 342 | 429.65 | 329.69 |
| 343 | 1760.83 | 2575.03 |
| 344 | 514.01 | 309.71 |
| 345 | 2515.14 | 115.68 |
| 346 | 565.18 | 631.08 |
| 347 | 348.79 | 312.62 |
| 348 | 184.84 | 126.88 |
| 349 | 1992.63 | 2743.26 |
| 350 | 4620.61 | 341.71 |
| 351 | 21183.3 | 33275.8 |
| 352 | 3541.13 | 4832.9 |
| 353 | 267.96 | 178.61 |
| 354 | 260.72 | 600.85 |
| 355 | 598.13 | 660.69 |
| 356 | 318.88 | 381.49 |
| 357 | 784.39 | 610.28 |
| 358 | 394.52 | 233.81 |
| 359 | 2575.84 | 1071.44 |
| 360 | 2460.15 | 3145.77 |
| 361 | 1463.89 | 405.51 |
| 362 | 330.7 | 296.81 |
| 363 | 430 | 232.16 |
| 364 | 550.02 | 691.79 |
| 365 | 1968.51 | 197.72 |
| 366 | 350.92 | 381.91 |
| 367 | 706.87 | 835.85 |
| 368 | 2384.33 | 175.16 |
| 369 | 3854.98 | 5258.01 |
| 370 | 14948.2 | 21376.3 |
| 371 | 728.16 | 250.02 |
| 372 | 552.5 | 144.36 |
| 373 | 210.05 | 246.6 |
| 374 | 347.41 | 249.22 |
| 375 | 207.87 | 315.21 |
| 376 | 357.91 | 878.46 |
| 377 | 274.83 | 200.69 |
| 378 | 852.25 | 552.92 |
| 379 | 427.78 | 628.41 |
| 380 | 344.12 | 480.68 |
| 381 | 10371.5 | 253.26 |
| 382 | 1974.82 | 2562.08 |
| 383 | 869.39 | 408.55 |
| 384 | 72.76 | 94.63 |
| 385 | 441.96 | 297.83 |
| 386 | 401.89 | 356.62 |
| 387 | 1259.94 | 1584.04 |
| 388 | 1178.53 | 2616.54 |
| 389 | 358.67 | 507.39 |
| 390 | 482.45 | 537.75 |
| 391 | 1542.53 | 2790.5 |
| 392 | 503.57 | 330.15 |
| 393 | 2430.83 | 3353.77 |
| 394 | 523.9 | 843.44 |
| 395 | 478.99 | 596.56 |
| 396 | 1207.17 | 1548.95 |
| 397 | 248.65 | 483.71 |
| 398 | 166.63 | 140.29 |
| 399 | 1049.78 | 696.02 |
| 400 | 3862.62 | 4198.48 |
| 401 | 733.5 | 999.31 |
| 402 | 5193.79 | 7951.13 |
| 403 | 2139.76 | 2730.17 |
| 404 | 916.36 | 816.75 |
| 405 | 1684.49 | 1580.08 |
| 406 | 237.78 | 343.53 |
| 407 | 2026.66 | 464.44 |
| 408 | 196.63 | 264.47 |
| 409 | 304.49 | 439.85 |
| 410 | 337.51 | 477.51 |
| 411 | 255.82 | 416.12 |
| 412 | 1221.53 | 1619.64 |
| 413 | 9331.13 | 14100.7 |
| 414 | 197.33 | 300.63 |
| 415 | 212 | 165.73 |
| 416 | 1346.36 | 1460.35 |
| 417 | 820.34 | 1192.4 |
| 418 | 1548.36 | 1967.46 |
| 419 | 1218.82 | 1006.41 |
| 420 | 218.44 | 251.93 |
| 421 | 667.59 | 467.84 |
| 422 | 630.62 | 817.36 |
| 423 | 1128.79 | 1580.15 |
| 424 | 584.6 | 641.55 |
| 425 | 923.66 | 1154.89 |
| 426 | 245.2 | 209.04 |
| 427 | 208.86 | 122.78 |
| 428 | 2141.81 | 2794.73 |
| 429 | 2673.32 | 3482.39 |
| 430 | 194.01 | 349.03 |
| 431 | 244.32 | 190.7 |
| 432 | 171.13 | 269.09 |
| 433 | 389.06 | 220.01 |
| 434 | 950.01 | 1103.05 |
| 435 | 737.26 | 1145.11 |
| 436 | 698.31 | 1006.16 |
| 437 | 1034.44 | 1328.66 |
| 438 | 1549.89 | 2565.32 |
| 439 | 242.37 | 346.97 |
| 440 | 4225.33 | 7163.11 |
| 441 | 2026.98 | 2749.89 |
| 442 | 1669.33 | 64.04 |
| 443 | 453.3 | 412.54 |
| 444 | 284.09 | 110.58 |
| 445 | 452.02 | 464.12 |
| 446 | 10928.25 | 6009.48 |
| 447 | 248.66 | 244.14 |
| 448 | 544.89 | 495.75 |
| 449 | 91.29 | 220.69 |
| 450 | 690.87 | 378.52 |
| 451 | 372.26 | 469.99 |
| 452 | 695.43 | 437.27 |
| 453 | 197.46 | 344.87 |
| 454 | 636.99 | 851.23 |
| 455 | 109.04 | 132.39 |
| 456 | 114.34 | 159.23 |
| 457 | 247.87 | 278.24 |
| 458 | 1565.61 | 2420.43 |
| 459 | 2149.51 | 2722.27 |
| 460 | 1761.85 | 1313.47 |
| 461 | 458.34 | 487.39 |
| 462 | 1518.75 | 1405.34 |
| 463 | 339.47 | 310.42 |
| 464 | 9070.76 | 1838.11 |
| 465 | 3861.14 | 2831.77 |
| 466 | 423.42 | 203.85 |
| 467 | 283.34 | 170.89 |
| 468 | 367.72 | 315.21 |
| 469 | 1424.84 | 339.86 |
| 470 | 1243.61 | 366.74 |
| 471 | 519.88 | 332.05 |
| 472 | 457.11 | 528.19 |
| 473 | 1906.66 | 2592.95 |
| 474 | 840.95 | 1804.57 |
| 475 | 285.04 | 113.11 |
| 476 | 232.38 | 117.03 |
| 477 | 4444.41 | 11160.44 |
| 478 | 698.61 | 390.57 |
| 479 | 648.4 | 507.64 |
| 480 | 442.16 | 240.49 |
| 481 | 16250.01 | 1560.5 |
| 482 | 945.07 | 559.31 |
| 483 | 1339.21 | 1514.58 |
| 484 | 358.02 | 192.21 |
| 485 | 232.09 | 187.49 |
| 486 | 684.31 | 479.07 |
| 487 | 728.45 | 407.72 |
| 488 | 196.89 | 476.3 |
| 489 | 633.98 | 149.83 |
| 490 | 91.61 | 147.08 |
| 491 | 502.32 | 282.04 |
| 492 | 441.16 | 507.47 |
| 493 | 714.01 | 210.4 |
| 494 | 349.59 | 153.45 |
| 495 | 269.05 | 450.84 |
| 496 | 403.2 | 515.62 |
| 497 | 513.8 | 133.24 |
| 498 | 295.46 | 431.46 |
| 499 | 790.03 | 915.09 |
| 500 | 411.21 | 61.58 |
| 501 | 1600.21 | 707.59 |
| 502 | 185.12 | 169.93 |
| 503 | 211.53 | 252.44 |
| 504 | 202.48 | 131.9 |
| 505 | 106.45 | 139.9 |
| 506 | 172.8 | 123.01 |
| 507 | 2238.19 | 1347.32 |
| 508 | 4881.51 | 210.22 |
| 509 | 386.33 | 241.64 |
| 510 | 389.85 | 197.63 |
| 511 | 1044.25 | 1860.09 |
| 512 | 911.8 | 213.78 |
| 513 | 165.39 | 235.62 |
| 514 | 366.71 | 78.89 |
| 515 | 3952.23 | 943.15 |
| 516 | 394.1 | 154.31 |
| 517 | 721.07 | 344.83 |
| 518 | 866.53 | 120.51 |
| 519 | 384.56 | 159.41 |
| 520 | 1491.26 | 589.3 |
| 521 | 969.57 | 410.04 |
| 522 | 1522.66 | 1334.42 |
| 523 | 145.86 | 141.48 |
| 524 | 189.68 | 287.18 |
| 525 | 1514.05 | 111.26 |
| 526 | 270.67 | 55.86 |
| 527 | 223.41 | 313.99 |
| 528 | 220.43 | 162.9 |
| 529 | 775.4 | 654.44 |
| 530 | 115.96 | 136.71 |
| 531 | 2682.19 | 2753.18 |
| 532 | 1004.59 | 725.68 |
| 533 | 24215.6 | 23635.89 |
| 534 | 1380.23 | 1722.54 |
| 535 | 635.5 | 466.98 |
| 536 | 590.85 | 530.1 |
| 537 | 205.17 | 252.33 |
| 538 | 262.07 | 133.39 |
| 539 | 236.94 | 716.14 |
| 540 | 116.27 | 110.6 |
| 541 | 286.2 | 368.78 |
| 542 | 355.35 | 522.74 |
| 543 | 3635.8 | 755.11 |
| 544 | 282.24 | 163.79 |
| 545 | 258.35 | 676.03 |
| 546 | 336.29 | 179.19 |
| 547 | 411.08 | 341.22 |
| 548 | 288.6 | 126.04 |
| 549 | 433.38 | 207.91 |
| 550 | 468.04 | 764.16 |
| 551 | 514.82 | 290.72 |
| 552 | 309.49 | 252.04 |
| 553 | 352.9 | 63.73 |
| 554 | 832.83 | 559.35 |
| 555 | 213.46 | 131.69 |
| 556 | 2691.55 | 211.72 |
| 557 | 5649.48 | 1095.48 |
| 558 | 267.74 | 274.93 |
| 559 | 197.06 | 351.22 |
| 560 | 322.71 | 181.52 |
| 561 | 1774.17 | 965.1 |
| 562 | 674.15 | 1019.16 |
| 563 | 514.89 | 202.92 |
| 564 | 280.12 | 231.27 |
| 565 | 612.61 | 484.55 |
| 566 | 305.8 | 281.04 |
| 567 | 6126.15 | 6394.03 |
| 568 | 564.34 | 127.81 |
| 569 | 895.57 | 671.47 |
| 570 | 234.83 | 74.14 |
| 571 | 918.01 | 306.21 |
| 572 | 490.15 | 215.39 |
| 573 | 340.36 | 561.18 |
| 574 | 293.37 | 165.07 |
| 575 | 1457.87 | 1267 |
| 576 | 186.86 | 193.04 |
| 577 | 15200.82 | 840.95 |
| 578 | 445 | 510.51 |
| 579 | 503.8 | 219.86 |
| 580 | 3363.04 | 542.25 |
| 581 | 399.54 | 283.96 |
| 582 | 1073.6 | 416.48 |
| 583 | 344.17 | 359.53 |
| 584 | 679.11 | 618.79 |
| 585 | 657.73 | 217.1 |
| 586 | 3920.85 | 58.99 |
| 587 | 192.77 | 218.19 |
| 588 | 417.28 | 103.5 |
| 589 | 331.42 | 405.5 |
| 590 | 2854 | 126.77 |
| 591 | 809.25 | 1010.64 |
| 592 | 262.72 | 539.84 |
| 593 | 146.88 | 411.88 |
| 594 | 8079.35 | 1105.07 |
| 595 | 488.35 | 132.06 |
| 596 | 3335 | 859.68 |
| 597 | 516.5 | 679.53 |
| 598 | 1181.54 | 168.71 |
| 599 | 411.12 | 893.15 |
| 600 | 297.54 | 266.17 |
| 601 | 1122.23 | 1242.63 |
| 602 | 140.12 | 176.5 |
| 603 | 2821 | 1369.9 |
| 604 | 894.97 | 169.59 |
| 605 | 342.43 | 207.05 |
| 606 | 5208.16 | 8436.04 |
| 607 | 109.36 | 126.02 |
| 608 | 5005.47 | 886.26 |
| 609 | 385.8 | 404.35 |
| 610 | 384.35 | 2017.29 |
| 611 | 1447.69 | 90.16 |
| 612 | 189.43 | 158.12 |
| 613 | 886.48 | 201.67 |
| 614 | 171.69 | 342.56 |
| 615 | 712.07 | 723.54 |
| 616 | 604.67 | 979.18 |
| 617 | 645.45 | 1001.2 |
| 618 | 367.72 | 477.68 |
| 619 | 188.76 | 457.6 |
| 620 | 612.89 | 94.01 |
| 621 | 225.78 | 223.31 |
| 622 | 3910.57 | 6523.87 |
| 623 | 258.4 | 256.33 |
| 624 | 693.23 | 913.29 |
| 625 | 92.22 | 168.82 |
| 626 | 338.07 | 497.04 |
| 627 | 1743.21 | 858.44 |
| 628 | 1934.06 | 274.04 |
| 629 | 3254.92 | 22.36 |
| 630 | 173.04 | 158.48 |
| 631 | 375.54 | 200.28 |
| 632 | 317.73 | 93 |
| 633 | 2739.3 | 1120.66 |
| 634 | 563.71 | 786.49 |
| 635 | 1199.43 | 277.21 |
| 636 | 744.12 | 919.2 |
| 637 | 346.57 | 197.87 |
| 638 | 1090.93 | 283.03 |
| 639 | 290.46 | 140.43 |
| 640 | 111.85 | 66.25 |
| 641 | 455.21 | 420.21 |
| 642 | 345.83 | 199.62 |
| 643 | 1651.16 | 1011.11 |
| 644 | 4741.83 | 614.65 |
| 645 | 697.29 | 334.7 |
| 646 | 277.24 | 288.32 |
| 647 | 4690.58 | 3358.31 |
| 648 | 240.79 | 294.91 |
| 649 | 641.99 | 200.78 |
| 650 | 371.35 | 238.75 |
| 651 | 2349.83 | 1291.14 |
| 652 | 193.38 | 143.66 |
| 653 | 1226.05 | 1935.03 |
| 654 | 5138.39 | 3725.55 |
| 655 | 3230.3 | 7444.98 |
| 656 | 746.95 | 386.71 |
| 657 | 239.61 | 201.28 |
| 658 | 464.77 | 869.33 |
| 659 | 210.32 | 980.59 |
| 660 | 3851.92 | 329.08 |
| 661 | 2485.76 | 2717 |
| 662 | 347.83 | 623.37 |
| 663 | 145.28 | 189.73 |
| 664 | 479.37 | 1413.9 |
| 665 | 690.96 | 407.33 |
| 666 | 440.32 | 770.9 |
| 667 | 348.99 | 416.59 |
| 668 | 321.33 | 612.28 |
| 669 | 330.69 | 333.64 |
| 670 | 465.29 | 768.38 |
| 671 | 804.23 | 897.03 |
| 672 | 331.26 | 117.39 |
| 673 | 18864.8 | 34.43 |
| 674 | 222.74 | 256.29 |
| 675 | 777.58 | 927.95 |
| 676 | 218.75 | 161.04 |
| 677 | 675.28 | 307.55 |
| 678 | 1427.9 | 1826.7 |
| 679 | 1500.77 | 1729 |
| 680 | 1179.77 | 1493.4 |
| 681 | 1035.25 | 621.15 |
| 682 | 827.45 | 539.76 |
| 683 | 928.3 | 504.81 |
| 684 | 379.72 | 200.01 |
| 685 | 454.16 | 274.58 |
| 686 | 1247.87 | 797.98 |
| 687 | 21125.9 | 178.01 |
| 688 | 131 | 156.14 |
| 689 | 86.47 | 120.23 |
| 690 | 184.62 | 196.27 |
| 691 | 326.21 | 74.96 |
| 692 | 467.89 | 307.96 |
| 693 | 796.96 | 485.58 |
| 694 | 2248.11 | 1306 |
| 695 | 492.82 | 227.88 |
| 696 | 443.67 | 725.02 |
| 697 | 810.12 | 535.52 |
| 698 | 998.77 | 1170 |
| 699 | 192.96 | 140.39 |
| 700 | 115 | 132.51 |
| 701 | 2241.77 | 2710.4 |
| 702 | 770.19 | 988.21 |
| 703 | 401.99 | 247.99 |
| 704 | 360.05 | 552.79 |
| 705 | 107.75 | 98.14 |
| 706 | 998.83 | 1032.7 |
| 707 | 164.07 | 209.94 |
| 708 | 1627.4 | 1118.7 |
| 709 | 763.49 | 250.07 |
| 710 | 773.85 | 675.53 |
| 711 | 155.22 | 286.79 |
| 712 | 1272.34 | 2512.1 |
| 713 | 27791.3 | 1191.1 |
| 714 | 459.25 | 281.17 |
| 715 | 941.28 | 1159 |
| 716 | 341.85 | 213.77 |
| 717 | 869.93 | 987.76 |
| 718 | 35304.9 | 419.08 |
| 719 | 295.28 | 457.92 |
| 720 | 97.18 | 91.86 |
| 721 | 5238.18 | 742.94 |
| 722 | 1105.15 | 2446.05 |
| 723 | 15994.6 | 18395.9 |
| 724 | 25574.2 | 56.87 |
| 725 | 223.22 | 334.29 |
| 726 | 390.33 | 591.38 |
| 727 | 874.08 | 1016.73 |
| 728 | 129.08 | 109.04 |
| 729 | 287.17 | 386.91 |
| 730 | 938.84 | 1689.36 |
| 731 | 249.13 | 373.29 |
| 732 | 2681.59 | 934.95 |
| 733 | 518.6 | 570.92 |
| 734 | 304.49 | 410.25 |
| 735 | 515.18 | 321.95 |
| 736 | 719.9 | 389.6 |
| 737 | 162.63 | 213.43 |
| 738 | 202.87 | 327.64 |
| 739 | 310.7 | 366.38 |
| 740 | 1057.06 | 967.69 |
| 741 | 1305.72 | 1525.5 |
| 742 | 406.27 | 251.43 |
| 743 | 209.98 | 332.04 |
| 744 | 779.02 | 507.03 |
| 745 | 509.21 | 780.22 |
| 746 | 550.52 | 205.81 |
| 747 | 303.67 | 178.71 |
| 748 | 391.6 | 345.85 |
| 749 | 161.04 | 486.26 |
| 750 | 1633.21 | 1883.97 |
| 751 | 1948.88 | 819.34 |
| 752 | 947.01 | 1410.71 |
| 753 | 88.29 | 103.94 |
| 754 | 748.98 | 398.24 |
| 755 | 5654.78 | 2358.14 |
| 756 | 176.88 | 284.44 |
| 757 | 641.35 | 134.74 |
| 758 | 193.83 | 250.67 |
| 759 | 265.87 | 443.61 |
| 760 | 1611.2 | 2134.8 |
| 761 | 895 | 953.08 |
| 762 | 4307.33 | 1318.11 |
| 763 | 425.51 | 320.2 |
| 764 | 157.27 | 248.54 |
| 765 | 2159.53 | 4308.89 |
| 766 | 601.24 | 540.73 |
| 767 | 259.57 | 190.8 |
| 768 | 268.04 | 788.11 |
| 769 | 1124.91 | 947.92 |
| 770 | 345.81 | 258.56 |
| 771 | 4076.84 | 585.19 |
| 772 | 1545.75 | 627.71 |
| 773 | 635.06 | 927.49 |
| 774 | 1015.92 | 550.41 |
| 775 | 475.86 | 490.03 |
| 776 | 5071.3 | 7395.4 |
| 777 | 271.15 | 292.43 |
| 778 | 502.9 | 356.61 |
| 779 | 4550.74 | 733.37 |
| 780 | 811.84 | 153.69 |
| 781 | 5268.58 | 6177 |
| 782 | 964.7 | 441.9 |
| 783 | 1288.41 | 1241.8 |
| 784 | 761.9 | 106.33 |
| 785 | 302.57 | 220.29 |
| 786 | 441.39 | 34.69 |
| 787 | 4805.19 | 408.09 |
| 788 | 724.85 | 750.37 |
| 789 | 2797.11 | 4381.9 |
| 790 | 1660.28 | 627.22 |
| 791 | 82.16 | 51.86 |
| 792 | 1500.15 | 777.08 |
| 793 | 131.83 | 184.79 |
| 794 | 1950.66 | 1314.3 |
| 795 | 445.08 | 140.52 |
| 796 | 421.99 | 182.5 |
| 797 | 426.27 | 390.57 |
| 798 | 498.29 | 564.91 |
| 799 | 483.58 | 288.48 |
| 800 | 177.85 | 148.57 |
| 801 | 1927.19 | 290.82 |
| 802 | 1908.71 | 378.98 |
| 803 | 1356.99 | 309.29 |
| 804 | 590.4 | 454.65 |
| 805 | 479.33 | 263.09 |
| 806 | 1688.17 | 334.94 |
| 807 | 2524.15 | 1194 |
| 808 | 613.82 | 295.85 |
| 809 | 1847.5 | 1489 |
| 810 | 551.98 | 311.34 |
| 811 | 908.48 | 388.74 |
| 812 | 124.16 | 66.69 |
| 813 | 1567.12 | 859.71 |
| 814 | 537.13 | 331.57 |
| 815 | 1081.92 | 743.86 |
| 816 | 1288.32 | 753.52 |
| 817 | 1522.33 | 1216.1 |
| 818 | 3743.12 | 844.55 |
| 819 | 211.47 | 155.57 |
| 820 | 232.71 | 324.57 |
| 821 | 311.5 | 454.92 |
| 822 | 433.81 | 689.88 |
| 823 | 2674.9 | 1470.8 |
| 824 | 2022.24 | 1507.3 |
| 825 | 4346.69 | 2618.9 |
| 826 | 2344.92 | 1552.8 |
| 827 | 898.51 | 575.57 |
| 828 | 901.65 | 505.99 |
| 829 | 1946.95 | 9898.6 |
| 830 | 961.96 | 4337.1 |
| 831 | 1561.13 | 2095.7 |
| 832 | 5309.3 | 1583.6 |
| 833 | 532.67 | 313.23 |
| 834 | 855.38 | 600.48 |
| 835 | 900.1 | 189.35 |
| 836 | 4098.86 | 2173.05 |

23. The method according to claim 1, wherein at least ten or 20 or 100 or all polypeptide markers as defined in claim 1 are used.

24. The method according to claim 1, wherein said sample from a subject is a urine sample or a blood sample (serum or plasma sample).

25. The method according to claim 1, wherein capillary electrophoresis, HPLC, gas-phase ion spectrometry and/or mass spectrometry is used for detecting the presence or absence or amplitude of the polypeptide marker or markers.

26. The method according to claim 1, wherein a capillary electrophoresis is performed before the molecular mass of the polypeptide markers is measured.

27. The method according to claim 1, wherein mass spectrometry is used for detecting the presence or absence or amplitude of the polypeptide marker or markers.

28. The method according to claim 1, wherein said method is used for the diagnosis of bladder cancer.

29. The method according to claim 1, wherein said method is used for determining a tumor stage of bladder cancer.

30. The method according to claim 1, wherein said method is used for the diagnosis of bladder cancer and for determining a tumor stage of bladder cancer simultaneously.

31. Use of at least one peptide marker selected from marker Nos. 1 to 836, which are characterized by the values according to claim 1 for the molecular mass and the migration time, for the diagnosis and/or for determining a tumor stage of bladder cancer.

32. A method for the diagnosis of bladder cancer (BC), comprising the steps of:
a) separating a sample into at least three, preferably 10, subsamples;
b) analyzing at least two subsamples for determining the presence or absence or amplitude of at least one polypeptide marker in a sample, wherein said polypeptide marker is selected from markers 1 to 836, which are characterized by the molecular masses and migration times (CE time) according to claim 1.

33. A method for determining a tumor stage of bladder cancer, comprising the steps of:
a) separating a sample into at least three, preferably 10, subsamples;
b) analyzing at least two subsamples for determining the presence or absence or amplitude of at least one polypeptide marker in a sample, wherein said polypeptide marker is selected from markers 1 to 836, which are characterized by the molecular masses and migration times (CE time) according to claim 1.

34. The method according to either of claims 14 or 15, wherein at least 10 subsamples are measured.

35. The method according to claim 1, wherein said CE time is based on a glass capillary of 90 cm in length and with an inner diameter (ID) of 50 μm at an applied voltage of 25 kV, wherein 20% acetonitrile, 0.25% formic acid in water is used as the mobile solvent.

36. A combination of markers, comprising at least 10 markers selected from markers 1 to 836, which are characterized by the molecular masses and migration times (CE time) according to claim 1.
